SEQUENCE LISTING (47 sequences)

SEQ ID NO: 1 - RNA, 23 nt, synthetic construct
  misc_feature 1..23: note = miR-135 mimetic oligos in-vitro
  modified_base 1: mod_base = OTHER; note = 5' phosphorylated
  misc_feature 23: note = 2'-O-Methyladenosine
  source 1..23: mol_type = other RNA
tatggctttttattcctatgtga

SEQ ID NO: 2 - RNA, 22 nt, synthetic construct
  misc_feature 1..22: note = miR-135 mimetic oligos in-vitro
  modified_base 1: mod_base = um
  modified_base 22: mod_base = gm
  source 1..22: mol_type = other RNA
  modified_base 2: mod_base = OTHER; note = 2'-O-Methyladenosine
tatagggattggagccgtggcg

SEQ ID NO: 3 - RNA, 22 nt, synthetic construct
  misc_feature 1..22: note = miR-135 mimetic oligos
  modified_base 1: mod_base = um
  modified_base 22: mod_base = gm
  source 1..22: mol_type = other RNA
  modified_base 2: mod_base = cm
tcatatagggattggagccgtg

SEQ ID NO: 4 - RNA, 23 nt, synthetic construct
  misc_feature 1..23: note = miR-135 mimetic oligos
  modified_base 1: mod_base = um
  modified_base 23: mod_base = OTHER; note = 2'-O-Methyladenosine
  source 1..23: mol_type = other RNA
  modified_base 2: mod_base = cm
tcacataggaataaaaagccata

SEQ ID NO: 5 - RNA, 23 nt, synthetic construct
  misc_feature 1..23: note = miR-135 mimetic oligos
  modified_base 1: mod_base = OTHER; note = 5' phosphorylated
  modified_base 10..11: mod_base = um
  modified_base 23: mod_base = OTHER; note = 2'-O-Methyladenosine
  source 1..23: mol_type = other RNA
tatggctttttattcctatgtga

SEQ ID NO: 6 - RNA, 22 nt, synthetic construct
  misc_feature 1..22: note = miR-135 mimetic oligos
  modified_base 1: mod_base = um
  modified_base 10: mod_base = um
  modified_base 22: mod_base = gm
  source 1..22: mol_type = other RNA
  modified_base 2: mod_base = OTHER; note = 2'-O-Methyladenosine
tatagggattggagccgtggcg

SEQ ID NO: 7 - RNA, 23 nt, synthetic construct
  misc_feature 1..23: note = miR-135 mimetic oligos
  modified_base 1: mod_base = OTHER; note = 5' phosphorylated
  modified_base 1: mod_base = um
  modified_base 4: mod_base = gm
  modified_base 23: mod_base = OTHER; note = 2'-O-Methyladenosine
  source 1..23: mol_type = other RNA
  modified_base 2: mod_base = OTHER; note = 2'-O-Methyladenosine
tatggctttttattcctatgtga

SEQ ID NO: 8 - RNA, 23 nt, synthetic construct
  misc_feature 1..23: note = miR-135 mimetic oligos
  modified_base 1: mod_base = OTHER; note = 5' phosphorylated
  modified_base 2: mod_base = OTHER; note = 2'-O-Methyladenosine
  modified_base 4: mod_base = um
  modified_base 23: mod_base = gm
  source 1..23: mol_type = other RNA
  modified_base 1: mod_base = um
taatttaagcttctttgttctgg

SEQ ID NO: 9 - RNA, 23 nt, synthetic construct
  misc_feature 1..23: note = miR-135 mimetic oligos
  modified_base 1: mod_base = cm
  modified_base 23: mod_base = OTHER; note = 2'-O-Methyladenosine
  source 1..23: mol_type = other RNA
ccagaacaaagaagcttaaatta

SEQ ID NO: 10 - RNA, 23 nt, synthetic construct
  misc_feature 1..23: note = miR-135 mimetic oligos
  modified_base 1: mod_base = OTHER; note = 5' phosphorylated
  modified_base 23: mod_base = OTHER; note = 2'-O-Methyladenosine
  source 1..23: mol_type = other RNA
tatggcttttcattcctatgtga

SEQ ID NO: 11 - RNA, 22 nt, synthetic construct
  misc_feature 1..22: note = miR-135 mimetic oligos
  modified_base 1: mod_base = OTHER; note = 2'-O-Methyladenosine
  modified_base 22: mod_base = gm
  source 1..22: mol_type = other RNA
  modified_base 2: mod_base = um
atgtagggctaaaagccatggg

SEQ ID NO: 12 - RNA, 22 nt, synthetic construct
  misc_feature 1..22: note = miR-135 mimetic oligos
  modified_base 1: mod_base = um
  source 1..22: mol_type = other RNA
  modified_base 2: mod_base = cm
tcatgtagggctaaaagccatg

SEQ ID NO: 13 - RNA, 23 nt, synthetic construct
  misc_feature 1..23: note = miR-135 mimetic oligos
  modified_base 1: mod_base = um
  misc_feature 1: note = Could be conjugated at 5 prime to sertraline or other cell targeting moiety
  modified_base 23: mod_base = OTHER; note = 2'-O-Methyladenosine
  source 1..23: mol_type = other RNA
  modified_base 2: mod_base = cm
tcacataggaatgaaaagccata

SEQ ID NO: 14 - RNA, 23 nt, synthetic construct
  misc_feature 1..23: note = miR-135 mimetic oligos
  modified_base 1: mod_base = OTHER; note = 5' phosphorylated
  modified_base 11: mod_base = cm
  modified_base 23: mod_base = OTHER; note = 2'-O-Methyladenosine
  source 1..23: mol_type = other RNA
  modified_base 12: mod_base = OTHER; note = 2'-O-Methyladenosine
  modified_base 13: mod_base = um
tatggcttttcattcctatgtga

SEQ ID NO: 15 - RNA, 22 nt, synthetic construct
  misc_feature 1..22: note = miR-135 mimetic oligos
  modified_base 1: mod_base = OTHER; note = 2'-O-Methyladenosine
  modified_base 9: mod_base = cm
  modified_base 22: mod_base = gm
  source 1..22: mol_type = other RNA
  modified_base 2: mod_base = um
  modified_base 10: mod_base = um
atgtagggctaaaagccatggg

SEQ ID NO: 16 - RNA, 23 nt, synthetic construct
  misc_feature 1..23: note = miR-135 mimetic oligos
  modified_base 1: mod_base = OTHER; note = 5' phosphorylated
  modified_base 1: mod_base = um
  modified_base 4: mod_base = gm
  modified_base 23: mod_base = OTHER; note = 2'-O-Methyladenosine
  source 1..23: mol_type = other RNA
  modified_base 2: mod_base = OTHER; note = 2'-O-Methyladenosine
tatggcttttcattcctatgtga

SEQ ID NO: 17 - DNA, 20 bp, synthetic construct
  misc_feature 1..20: note = Single strand DNA oligonucleotide
  source 1..20: mol_type = other DNA
atccgcatgaatgctgtgta

SEQ ID NO: 18 - DNA, 20 bp, synthetic construct
  misc_feature 1..20: note = Single strand DNA oligonucleotide
  source 1..20: mol_type = other DNA
gtgggtggtggaagagacac

SEQ ID NO: 19 - DNA, 19 bp, synthetic construct
  misc_feature 1..19: note = Single strand DNA oligonucleotide
  source 1..19: mol_type = other DNA
agttctgccgctgatgatg

SEQ ID NO: 20 - DNA, 24 bp, synthetic construct
  misc_feature 1..24: note = Single strand DNA oligonucleotide
  source 1..24: mol_type = other DNA
gcacaaatggagagtctgattaaa

SEQ ID NO: 21 - DNA, 23 bp, synthetic construct
  misc_feature 1..23: note = Single strand DNA oligonucleotide
  source 1..23: mol_type = other DNA
tatggctttttattcctatgtga

SEQ ID NO: 22 - DNA, 23 bp, synthetic construct
  misc_feature 1..23: note = Single strand DNA oligonucleotide
  source 1..23: mol_type = other DNA
tatggcttttcattcctatgtga

SEQ ID NO: 23 - DNA, 22 bp, synthetic construct
  misc_feature 1..22: note = Single strand DNA oligonucleotide
  source 1..22: mol_type = other DNA
tttgttcgttcggctcgcgtga

SEQ ID NO: 24 - DNA, 21 bp, synthetic construct
  misc_feature 1..21: note = Single strand DNA oligonucleotide
  source 1..21: mol_type = other DNA
gatgacacgcaaattcgtgaa

SEQ ID NO: 25 - DNA, 20 bp, synthetic construct
  misc_feature 1..20: note = Single strand DNA oligonucleotide
  source 1..20: mol_type = other DNA
taaggcacgcggtgaatgcc

SEQ ID NO: 26 - DNA, 22 bp, synthetic construct
  misc_feature 1..22: note = Single strand DNA oligonucleotide
  source 1..22: mol_type = other DNA
tagcagcacgtaaatattggcg

SEQ ID NO: 27 - DNA, 22 bp, synthetic construct
  misc_feature 1..22: note = Single strand DNA oligonucleotide
  source 1..22: mol_type = other DNA
gggtttggatagtacgttcgca

SEQ ID NO: 28 - DNA, 20 bp, synthetic construct
  misc_feature 1..20: note = Single strand DNA oligonucleotide
  source 1..20: mol_type = other DNA
catacgcccctcctgatgtc

SEQ ID NO: 29 - DNA, 20 bp, synthetic construct
  misc_feature 1..20: note = Single strand DNA oligonucleotide
  source 1..20: mol_type = other DNA
gtgcaccatcagcaaggacc

SEQ ID NO: 30 - DNA, 20 bp, synthetic construct
  misc_feature 1..20: note = Single strand DNA oligonucleotide
  source 1..20: mol_type = other DNA
gcgccgaaagtggagtagat

SEQ ID NO: 31 - DNA, 21 bp, synthetic construct
  misc_feature 1..21: note = Single strand DNA oligonucleotide
  source 1..21: mol_type = other DNA
tgacactggcaaaacaatgca

SEQ ID NO: 32 - DNA, 21 bp, synthetic construct
  misc_feature 1..21: note = Single strand DNA oligonucleotide
  source 1..21: mol_type = other DNA
ggtccttttcaccagcaagct

SEQ ID NO: 33 - DNA, 20 bp, synthetic construct
  misc_feature 1..20: note = Single strand DNA oligonucleotide
  source 1..20: mol_type = other DNA
tgcaccaccaactgcttagc

SEQ ID NO: 34 - DNA, 21 bp, synthetic construct
  misc_feature 1..21: note = Single strand DNA oligonucleotide
  source 1..21: mol_type = other DNA
ggcatggactgtggtcatgag

SEQ ID NO: 35 - DNA, 24 bp, synthetic construct
  misc_feature 1..24: note = Single strand DNA oligonucleotide
  source 1..24: mol_type = other DNA
acttttggtacattgtggcttcaa

SEQ ID NO: 36 - DNA, 20 bp, synthetic construct
  misc_feature 1..20: note = Single strand DNA oligonucleotide
  source 1..20: mol_type = other DNA
ccgccaggacaaaccagtat

SEQ ID NO: 37 - RNA, 23 nt, synthetic construct
  misc_feature 1..23: note = mature miR-135b
  source 1..23: mol_type = other RNA
tatggcttttcattcctatgtga

SEQ ID NO: 38 - RNA, 22 nt, synthetic construct
  misc_feature 1..22: note = mature miR-135b*
  source 1..22: mol_type = other RNA
atgtagggctaaaagccatggg

SEQ ID NO: 39 - RNA, 97 nt, synthetic construct
  misc_feature 1..97: note = miR-135b microRNA precursor
  source 1..97: mol_type = other RNA
cactctgctgtggcctatggcttttcattcctatgtgattgctgtcccaaactcatgtagggctaaaagccatgggctacagtgaggggcgagctcc

SEQ ID NO: 40 - RNA, 23 nt, synthetic construct
  misc_feature 1..23: note = RNA oligonucleotide complementary to miR-135b
  source 1..23: mol_type = other RNA
tcacataggaatgaaaagccata

SEQ ID NO: 41 - RNA, 24 nt, synthetic construct
  misc_feature 1..24: note = miR-135 mimetic oligos
  misc_feature 1: note = 5' phosphorylated
  modified_base 1: mod_base = OTHER; note = 2'-O-methoxyethyl Uracil modification (2-O-MOE-5-Me)
  modified_base 24: mod_base = OTHER; note = 2'-O-Methyladenosine
  source 1..24: mol_type = other RNA
ttatggcttttcattcctatgtga

SEQ ID NO: 42 - RNA, 26 nt, synthetic construct
  misc_feature 1..26: note = miR-135 mimetic oligos
  modified_base 1: mod_base = OTHER; note = 5' phosphorylated
  modified_base 1: mod_base = OTHER; note = 2-O-MOE-5-Me Uracil modification
  modified_base 24: mod_base = OTHER; note = 2'-O-Methyladenosine
  modified_base 25..26: mod_base = OTHER; note = 2'-O-MOE Adenine modification
  modified_base 25^26: mod_base = OTHER; note = phosphorothioate bond
  source 1..26: mol_type = other RNA
ttatggcttttcattcctatgtgaaa

SEQ ID NO: 43 - RNA, 26 nt, synthetic construct
  misc_feature 1..26: note = miR-135 mimetic oligos
  modified_base 1: mod_base = OTHER; note = 5' phosphorylated
  modified_base 1: mod_base = OTHER; note = 2-O-MOE-5-Me Uracil modification
  modified_base 2: mod_base = OTHER; note = 2'-fluoro
  modified_base 2: mod_base = OTHER; note = 2'-fluoro
  modified_base 3: mod_base = OTHER; note = 2'-O-Methyladenosine
  modified_base 4: mod_base = OTHER; note = 2'-fluoro
  modified_base 5: mod_base = gm
  modified_base 6: mod_base = OTHER; note = 2'-fluoro
  modified_base 7: mod_base = cm
  modified_base 8: mod_base = OTHER; note = 2'-fluoro
  modified_base 9: mod_base = um
  modified_base 10: mod_base = OTHER; note = 2'-fluoro
  modified_base 11: mod_base = um
  modified_base 12: mod_base = OTHER; note = 2'-fluoro
  modified_base 13: mod_base = OTHER; note = 2'-O-Methyladenosine
  modified_base 14: mod_base = OTHER; note = 2'-fluoro
  modified_base 15: mod_base = um
  modified_base 16: mod_base = OTHER; note = 2'-fluoro
  modified_base 17: mod_base = cm
  modified_base 18: mod_base = OTHER; note = 2'-fluoro
  modified_base 19: mod_base = OTHER; note = 2'-O-Methyladenosine
  modified_base 20: mod_base = OTHER; note = 2'-fluoro
  modified_base 21: mod_base = gm
  modified_base 22: mod_base = OTHER; note = 2'-fluoro
  modified_base 23: mod_base = gm
  modified_base 25..26: mod_base = OTHER; note = 2'-O-MOE Adenine modification
  modified_base 25^26: mod_base = OTHER; note = phosphorothioate bond
  source 1..26: mol_type = other RNA
  modified_base 24: mod_base = OTHER; note = 2'-O-Methyladenosine
ttatggcttttcattcctatgtgaaa

SEQ ID NO: 44 - RNA, 26 nt, synthetic construct
  misc_feature 1..26: note = miR-135 mimetic oligos
  modified_base 1: mod_base = OTHER; note = 5' phosphorylated
  modified_base 1: mod_base = OTHER; note = 2-O-MOE-5-Me Uracil modification
  modified_base 1^2: mod_base = OTHER; note = phosphorothioate bond
  modified_base 2: mod_base = OTHER; note = 2'-fluoro
  modified_base 2: mod_base = OTHER; note = 2'-fluoro
  modified_base 3: mod_base = OTHER; note = 2'-O-Methyladenosine
  modified_base 4: mod_base = OTHER; note = 2'-fluoro
  modified_base 4^5: mod_base = OTHER; note = phosphorothioate bond
  modified_base 5: mod_base = gm
  modified_base 6: mod_base = OTHER; note = 2'-fluoro
  modified_base 6^7: mod_base = OTHER; note = phosphorothioate bond
  modified_base 7: mod_base = cm
  modified_base 8: mod_base = OTHER; note = 2'-fluoro
  modified_base 8^9: mod_base = OTHER; note = phosphorothioate bond
  modified_base 9: mod_base = um
  modified_base 10: mod_base = OTHER; note = 2'-fluoro
  modified_base 10^11: mod_base = OTHER; note = phosphorothioate bond
  modified_base 11: mod_base = um
  modified_base 12: mod_base = OTHER; note = 2'-fluoro
  modified_base 12^13: mod_base = OTHER; note = phosphorothioate bond
  modified_base 13: mod_base = OTHER; note = 2'-O-Methyladenosine
  modified_base 14: mod_base = OTHER; note = 2'-fluoro
  modified_base 14^15: mod_base = OTHER; note = phosphorothioate bond
  modified_base 15: mod_base = um
  modified_base 16: mod_base = OTHER; note = 2'-fluoro
  modified_base 17: mod_base = cm
  modified_base 18: mod_base = OTHER; note = 2'-fluoro
  modified_base 19: mod_base = OTHER; note = 2'-O-Methyladenosine
  modified_base 20: mod_base = OTHER; note = 2'-fluoro
  modified_base 21: mod_base = gm
  modified_base 22: mod_base = OTHER; note = 2'-fluoro
  modified_base 23: mod_base = gm
  modified_base 25..26: mod_base = OTHER; note = 2'-O-MOE Adenine modification
  source 1..26: mol_type = other RNA
  modified_base 2^3: mod_base = OTHER; note = phosphorothioate bond
  modified_base 15^16: mod_base = OTHER; note = phosphorothioate bond
  modified_base 16^17: mod_base = OTHER; note = phosphorothioate bond
  modified_base 17^18: mod_base = OTHER; note = phosphorothioate bond
  modified_base 18^19: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 21^22: mod_base = OTHER; note = phosphorothioate bond
  modified_base 22^23: mod_base = OTHER; note = phosphorothioate bond
  modified_base 23^24: mod_base = OTHER; note = phosphorothioate bond
  modified_base 24^25: mod_base = OTHER; note = phosphorothioate bond
  modified_base 25^26: mod_base = OTHER; note = phosphorothioate bond
  modified_base 24: mod_base = OTHER; note = 2'-O-Methyladenosine
ttatggcttttcattcctatgtgaaa

SEQ ID NO: 45 - RNA, 26 nt, synthetic construct
  misc_feature 1..26: note = miR-135 mimetic oligos
  modified_base 1: mod_base = OTHER; note = 5' phosphorylated
  modified_base 1: mod_base = OTHER; note = 2-O-MOE-5-Me Uracil modification
  modified_base 1^2: mod_base = OTHER; note = phosphorothioate bond
  modified_base 2: mod_base = OTHER; note = 2'-fluoro
  modified_base 4^5: mod_base = OTHER; note = phosphorothioate bond
  modified_base 6^7: mod_base = OTHER; note = phosphorothioate bond
  modified_base 8^9: mod_base = OTHER; note = phosphorothioate bond
  modified_base 10^11: mod_base = OTHER; note = phosphorothioate bond
  modified_base 10: mod_base = OTHER; note = 2'-fluoro
  modified_base 11: mod_base = um
  modified_base 12^13: mod_base = OTHER; note = phosphorothioate bond
  modified_base 14^15: mod_base = OTHER; note = phosphorothioate bond
  modified_base 15: mod_base = um
  modified_base 16: mod_base = OTHER; note = 2'-fluoro
  modified_base 17: mod_base = cm
  modified_base 18: mod_base = OTHER; note = 2'-fluoro
  modified_base 19: mod_base = OTHER; note = 2'-O-Methyladenosine
  modified_base 20: mod_base = OTHER; note = 2'-fluoro
  modified_base 21: mod_base = gm
  modified_base 22: mod_base = OTHER; note = 2'-fluoro
  modified_base 23: mod_base = gm
  modified_base 25..26: mod_base = OTHER; note = 2'-O-MOE Adenine modification
  source 1..26: mol_type = other RNA
  modified_base 24: mod_base = OTHER; note = 2'-O-Methyladenosine
  modified_base 2^3: mod_base = OTHER; note = phosphorothioate bond
  modified_base 15^16: mod_base = OTHER; note = phosphorothioate bond
  modified_base 16^17: mod_base = OTHER; note = phosphorothioate bond
  modified_base 17^18: mod_base = OTHER; note = phosphorothioate bond
  modified_base 18^19: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 21^22: mod_base = OTHER; note = phosphorothioate bond
  modified_base 22^23: mod_base = OTHER; note = phosphorothioate bond
  modified_base 23^24: mod_base = OTHER; note = phosphorothioate bond
  modified_base 24^25: mod_base = OTHER; note = phosphorothioate bond
  modified_base 25^26: mod_base = OTHER; note = phosphorothioate bond
ttatggcttttcattcctatgtgaaa

SEQ ID NO: 46 - RNA, 26 nt, synthetic construct
  misc_feature 1..26: note = miR-135 mimetic oligos
  modified_base 1: mod_base = OTHER; note = 5' phosphorylated
  modified_base 1: mod_base = OTHER; note = 2-O-MOE-5-Me Uracil modification
  modified_base 1^2: mod_base = OTHER; note = phosphorothioate bond
  modified_base 2: mod_base = OTHER; note = 2'-fluoro
  modified_base 10^11: mod_base = OTHER; note = phosphorothioate bond
  modified_base 10: mod_base = OTHER; note = 2'-fluoro
  modified_base 11: mod_base = um
  modified_base 15^16: mod_base = OTHER; note = phosphorothioate bond
  modified_base 15: mod_base = um
  modified_base 16: mod_base = OTHER; note = 2'-fluoro
  modified_base 17: mod_base = cm
  modified_base 18: mod_base = OTHER; note = 2'-fluoro
  modified_base 19: mod_base = OTHER; note = 2'-O-Methyladenosine
  modified_base 20: mod_base = OTHER; note = 2'-fluoro
  modified_base 21: mod_base = gm
  modified_base 22: mod_base = OTHER; note = 2'-fluoro
  modified_base 24: mod_base = OTHER; note = 2'-O-Methyladenosine
  modified_base 25..26: mod_base = OTHER; note = 2'-O-MOE Adenine modification
  source 1..26: mol_type = other RNA
  modified_base 23: mod_base = gm
  modified_base 16^17: mod_base = OTHER; note = phosphorothioate bond
  modified_base 17^18: mod_base = OTHER; note = phosphorothioate bond
  modified_base 18^19: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 21^22: mod_base = OTHER; note = phosphorothioate bond
  modified_base 22^23: mod_base = OTHER; note = phosphorothioate bond
  modified_base 23^24: mod_base = OTHER; note = phosphorothioate bond
  modified_base 24^25: mod_base = OTHER; note = phosphorothioate bond
  modified_base 25^26: mod_base = OTHER; note = phosphorothioate bond
ttatggcttttcattcctatgtgaaa

SEQ ID NO: 47 - RNA, 23 nt, synthetic construct
  misc_feature 1..23: note = miR-135 mimetic oligos
  modified_base 1: mod_base = um
  misc_feature 1: note = Could be conjugated at 5 prime to sertraline or other cell targeting moiety
  modified_base 1^2: mod_base = OTHER; note = phosphorothioate bond
  modified_base 22^23: mod_base = OTHER; note = phosphorothioate bond
  modified_base 23: mod_base = OTHER; note = 2'-O-Methyladenosine
  source 1..23: mol_type = other RNA
  modified_base 21^22: mod_base = OTHER; note = phosphorothioate bond
  modified_base 2: mod_base = cm
  modified_base 2^3: mod_base = OTHER; note = phosphorothioate bond
tcacataggaatgaaaagccata

(19) United States
(12) Patent Application Publication
(10) Pub. No.: US 2024/0026357 A1
(43) Pub. Date: Jan. 25, 2024

(54) MODIFIED MIR-135, CONJUGATED FORM THEREOF, AND USES OF SAME

(71) Applicants: Yeda Research and Development Co. Ltd., Rehovot (IL); miCure Therapeutics Ltd., Tel Aviv (IL)

(72) Inventors: Alon CHEN, Rehovot (IL); Sharon MANASHIROV, Rehovot (IL); Andres Pablo MONTEFELTRO, Erts (AD)

(73) Assignees: Yeda Research and Development Co. Ltd., Rehovot (IL); miCure Therapeutics Ltd., Tel Aviv (IL)

(21) Appl. No.: 18/221,914

(22) Filed: Jul. 14, 2023

Related U.S. Application Data
(63) Continuation of application No. PCT/IL2022/050075, filed on Jan. 18, 2022.
(60) Provisional application No. 63/138,555, filed on Jan. 18, 2021.
(60) Provisional application No. 63/272,329, filed on Oct. 27, 2021.

(51) Int. Cl. C12N 15/113; A61K 47/55; A61K 47/54; A61P 25/24

(52) U.S. Cl. CPC C12N 15/113; A61K 47/55; A61K 47/548; A61P 25/24; C12N 2310/141

(57) ABSTRACT

A composition of matter comprising a synthetic miR-135 molecule comprising a nucleic acid sequence of a miR-135b as set forth in SEQ ID NO: 37, and a complementary strand as set forth in SEQ ID NO: 40 is disclosed. A conjugate comprising a composition of matter comprising a synthetic miR-135 molecule and a cell-targeting moiety is also disclosed.

### RELATED APPLICATIONS

This application is a Continuation of PCT Patent Application No. PCT/IL2022/050075 having International filing date of Jan. 18, 2022, which claims the benefit of priority of U.S. Provisional Patent Application No. 63/138,555 filed on Jan. 18, 2021 and U.S. Provisional Patent Application No. 63/272,329 filed on Oct. 27, 2021. The contents of the above applications are all incorporated by reference as if fully set forth herein in their entirety.

### SEQUENCE LISTING STATEMENT

The XML file, entitled 97025SequenceListing.xml, created on Jul. 14, 2023, comprising 159,960 bytes, submitted concurrently with the filing of this application is incorporated herein by reference.

### FIELD AND BACKGROUND OF THE INVENTION

The present invention, in some embodiments thereof, relates to therapeutic miR-135 molecules and, more particularly, but not exclusively, to the use of same.

Mood disorders, such as major depression, and anxiety disorders represent some of the most common and proliferating health problems worldwide effecting about 10% of the population. Despite many decades of research, the mechanisms behind depression onset, susceptibility and available therapies are only partially understood. Currently only about a third of patients respond to available treatments, therefore, there is a great need for better understanding of the pathology.

The current dogma regarding the etiology of depression is of a complex interaction between environmental factors and genetic predisposition, suggesting a mechanistic role for epigenetic processes.

Serotonin (5HT) is a monoamine neurotransmitter produced in the brain by the raphe nucleus (RN), which project extensively throughout the brain to modulate variety of cognitive, emotional and physiological functions. The link between dysregulated serotonergic activity and depression is well established [Michelsen K A. et al., Brain Res Rev (2007) 55(2):329-42]. The levels of 5HT, as well as the genetic circuitry in charge of it production, secretion, reuptake and deactivating, are dysregulated in depression. Furthermore, most currently available antidepressant drugs target the function of 5HT system related proteins, resulting in increased 5HT levels in the synapse [Krishnan V and Nestler E J, Nature (2008) 455: 894-902]. Available therapeutics require a long period of administration before relief of symptoms is observed.

MicroRNAs (miRs) are a subset of endogenous small (approximately 22 nucleotide) noncoding RNA molecules that repress gene expression post-transcriptionally. MiRs are transcribed as primary-miR molecules that are processed in the cell nucleus into precursor miRs with stem loop structures, which are exported to the cytoplasm where they are further processed into the active mature miRs. The mature miR is subsequently incorporated into the RNA-induced silencing complex and function primarily by binding to the 3′untranslated regions (3′UTRs) of specific mRNA molecules. Binding occurs via the seed sequence, a 6-8 nucleotides sequence at the 5′ end of the miR, that base pairs to a complementary seed match sequence on the target mRNA 3′ UTR. Binding of a miR leads to direct mRNA destabilization or translational repression, ultimately resulting in reduced protein levels of target gene.

MiRs are abundant in the nervous system, and initial research has mainly focused on neurons in the context of development, cancer and neurodegenerative disorders and normal process such as plasticity [Kosik K S. Nat Rev Neurosci (2006) 7:911-20]. Several miR-screening studies have reported that miR levels in various adult rodents or human brain structures are affected by a range of behavioral and pharmacological manipulations [O'Connor R. M. et al., Mol Psychiatry (2012) 17: 359-376]. Additionally, it has been suggested that miRs play a role in psychiatric disorders such as schizophrenia, autism and also depression and anxiety, both in humans and in mouse models [Miller B H and Wahlestedt C, Brain Res (2010) 1338: 89-99 and Issler O and Chen A, Nat Rev Neurosci. (2015) 16: 201-212]. Several studies have recently demonstrated the involvement of miRs in regulating 5HT related genes [Millan M J. Curr Opin Pharmacol (2011) 11(1):11-22] revealing the emerging role of miRs in the regulation of 5HT system and their potential association with depression related disorders.

PCT publication no. WO 2013/018060 discloses microRNAs (e.g. miR-135) and compositions comprising same for the treatment and diagnosis of serotonin-, adrenalin-, noradrenalin-, glutamate-, and corticotropin-releasing hormone-associated medical conditions.

PCT publication no. WO 2015/118537 discloses methods of treating a bipolar disorder by administering to the subject a therapeutically effective amount of a miR-135, a precursor thereof or a nucleic acid molecule encoding the miR-135 or the precursor thereof.

U.S. Patent Application No. 20170037404 discloses methods and compositions for introducing miRNA activity or function into cells using synthetic nucleic acid molecules. The synthetic nucleic acid molecules may be modified.

PCT publication no. WO/2011/131693 discloses a conjugate comprising (i) a nucleic acid which is complementary to a target nucleic acid sequence and which expression prevents or reduces expression of the target nucleic acid (e.g. miRNA) and (ii) a selectivity agent which is capable of binding with high affinity to a neurotransmitter transporter (e.g. serotonin reuptake inhibitors). WO/2011/131693 contemplates the use of these conjugates for delivery of nucleic acids to a cell of interest, for the treatment of diseases, as well as for diagnostic purposes.

### SUMMARY OF THE INVENTION

According to an aspect of some embodiments of the present invention there is provided a composition of matter comprising a synthetic miR-135 molecule comprising a nucleic acid sequence of a miR-135b as set forth in SEQ ID NO: 37, and a complementary strand as set forth in SEQ ID NO: 40.

According to an aspect of some embodiments of the present invention there is provided a composition of matter comprising a synthetic miR-135 molecule comprising a nucleic acid sequence of a miR-135b as set forth in any one of SEQ ID NOs: 10, 16 or 41-46, and a complementary strand as set forth in any one of SEQ ID NOs: 13 or 47.

According to an aspect of some embodiments of the present invention there is provided a composition of matter comprising a nucleic acid construct of the synthetic miR-135 molecule of some embodiments of the invention.

According to an aspect of some embodiments of the present invention there is provided a conjugate comprising:
(i) a composition of matter comprising the synthetic miR-135 molecule of some embodiments of the invention; and
(ii) a cell-targeting moiety.

According to an aspect of some embodiments of the present invention there is provided a pharmaceutical composition comprising the composition of matter of some embodiments of the invention, or the conjugate of some embodiments of the invention, and a pharmaceutically acceptable carrier.

According to an aspect of some embodiments of the present invention there is provided a method of treating a central nervous system (CNS)-related condition in a subject in need thereof, the method comprising administering to the subject a therapeutically effective amount of the composition of matter of some embodiments of the invention, or the conjugate of some embodiments of the invention, thereby treating the CNS-related condition.

According to an aspect of some embodiments of the present invention there is provided a method of treating a depression-related disorder in a subject in need thereof, the method comprising administering to the subject a therapeutically effective amount of the composition of matter of some embodiments of the invention, or the conjugate of some embodiments of the invention, thereby treating the depression-related disorder.

According to an aspect of some embodiments of the present invention there is provided a method of treating a cancerous disease in a subject in need thereof, the method comprising administering to the subject a therapeutically effective amount of the composition of matter of some embodiments of the invention, or the conjugate of some embodiments of the invention, thereby treating the cancerous disease.

According to an aspect of some embodiments of the present invention there is provided a method of promoting bone regeneration or muscle cell differentiation in a subject in need thereof, the method comprising administering to the subject a therapeutically effective amount of the composition of matter of some embodiments of the invention, or the conjugate of some embodiments of the invention, thereby promoting bone regeneration or muscle cell differentiation.

According to an aspect of some embodiments of the present invention there is provided a therapeutically effective amount of the composition of matter of some embodiments of the invention, or the conjugate of some embodiments of the invention, for use in treating a CNS-related condition in a subject in need thereof.

According to an aspect of some embodiments of the present invention there is provided a therapeutically effective amount of the composition of matter of some embodiments of the invention, or the conjugate of some embodiments of the invention, for use in treating a depression-related disorder in a subject in need thereof.

According to an aspect of some embodiments of the present invention there is provided a therapeutically effective amount of the composition of matter of some embodiments of the invention, or the conjugate of some embodiments of the invention, for use in treating a cancerous disease in a subject in need thereof.

According to an aspect of some embodiments of the present invention there is provided a therapeutically effective amount of the composition of matter of some embodiments of the invention, or the conjugate of some embodiments of the invention, for use in treating a bone-related disease or condition or a muscle-related disease or condition in a subject in need thereof.

According to some embodiments of the invention, the miR-135 molecule comprises no more than 50 nucleic acids.

According to some embodiments of the invention, the nucleic acid sequence of the miR-135b as set forth in SEQ ID NO: 37 and the complementary strand as set forth in SEQ ID NO: 40 are on separate nucleic acid sequence molecules that form the double stranded synthetic miR-135 molecule.

According to some embodiments of the invention, the nucleic acid sequence of the miR-135b as set forth in SEQ ID NO: 37 and the complementary strand as set forth in SEQ ID NO: 40 form a hairpin loop structure.

According to some embodiments of the invention, the nucleic acid sequence of the miR-135b as set forth in SEQ ID NO: 37 and the complementary strand as set forth in SEQ ID NO: 40 are 100% complementary over the entire length of SEQ ID NO: 37 and SEQ ID NO: 40.

According to some embodiments of the invention, the nucleic acid sequence of the miR-135b and/or the complementary strand comprises one or more modification selected from the group consisting of a sugar modification, a nucleobase modification, and an internucleotide linkage modification.

According to some embodiments of the invention, the sugar modification is selected from the group consisting of a 2′-O-methyl (2′-O-Me), a 2′-O-methoxyethyl (2′-O-MOE), a 2′-fluoro (2′-F), a locked nucleic acid (LNA), and a 2′-Fluoroarabinooligonucleotides (FANA).

According to some embodiments of the invention, the sugar modification in the miR-135b is in at least one nucleotide at the 3′ end of the nucleic acid sequence.

According to some embodiments of the invention, the sugar modification in the miR-135b is in at least one nucleotide at the 5′ end of the nucleic acid sequence.

According to some embodiments of the invention, the sugar modification in the complementary strand comprises a modification in the last nucleotide at the 3′ end of the nucleic acid sequence.

According to some embodiments of the invention, the sugar modification in the complementary strand comprises a modification in the first two nucleotides at the 5′ end of the nucleic acid sequence.

According to some embodiments of the invention, the sugar modification is a 2′-O-methyl (2′-O-Me), a 2′-O-methoxyethyl (2′-O-MOE), and/or a 2′-fluoro (2′-F) modification.

According to some embodiments of the invention, the sugar modification is a 2′-O-methoxyethyl (2′-O-MOE) and a 5′ ribose methylation (2′-O-MOE-5′-Me).

According to some embodiments of the invention, the internucleotide linkage modification is selected from the group consisting of a phosphorothioate, a chiral phosphorothioate, a phosphorodithioate, a phosphotriester, an aminoalkyl phosphotriester, a methyl phosphonate, an alkyl phosphonate, a chiral phosphonate, a phosphinate, a phosphoramidate, an aminoalkylphosphoramidate, a thionophosphoramidate, a thionoalkylphosphonate, a thionoalkylphosphotriester, a boranophosphate, a phosphodiester, a phosphonoacetate (PACE) and a peptide nucleic acid (PNA).

According to some embodiments of the invention, the internucleotide linkage modification in the miR-135b is in the last nucleotide at the 5′ end of the nucleic acid sequence.

According to some embodiments of the invention, the internucleotide linkage modification comprises a phosphate.

According to some embodiments of the invention, the internucleotide linkage modification comprises a phosphorothioate.

According to some embodiments of the invention, the phosphorothioate modification is between the last two nucleotides at the 3′ end of the nucleic acid sequence of the miR-135b or of the complementary strand.

According to some embodiments of the invention, the phosphorothioate modification is between the last two nucleotides at the 5′ end of the nucleic acid sequence of the miR-135b or of the complementary strand.

According to some embodiments of the invention, the nucleic acid sequence of the miR-135b comprising the modification is as set forth in any one of SEQ ID NOs: 10, 16 or 41-46.

According to some embodiments of the invention, the nucleic acid sequence of the complementary strand comprising the modification is as set forth in any one of SEQ ID NOs: 13 or 47.

According to some embodiments of the invention, the nucleic acid sequence of the miR-135b is as set forth in SEQ ID NO: 10 and the complementary strand is as set forth in SEQ ID NO: 13.

According to some embodiments of the invention, the nucleic acid sequence of the miR-135b is as set forth in SEQ ID NO: 41 and the complementary strand is as set forth in SEQ ID NO: 13.

According to some embodiments of the invention, the nucleic acid sequence of the miR-135b is as set forth in SEQ ID NO: 42 and the complementary strand is as set forth in SEQ ID NO: 13.

According to some embodiments of the invention, the nucleic acid sequence of the miR-135b is as set forth in SEQ ID NO: 10 and the complementary strand is as set forth in SEQ ID NO: 47.

According to some embodiments of the invention, the nucleic acid sequence of the miR-135b is as set forth in SEQ ID NO: 16 and the complementary strand is as set forth in SEQ ID NO: 13.

According to some embodiments of the invention, the nucleic acid sequence of the miR-135b is as set forth in SEQ ID NO: 41 and the complementary strand is as set forth in SEQ ID NO: 47.

According to some embodiments of the invention, the nucleic acid sequence of the miR-135b is as set forth in SEQ ID NO: 42 and the complementary strand is as set forth in SEQ ID NO: 47.

According to some embodiments of the invention, the nucleic acid sequence of the miR-135b as set forth in any one of SEQ ID NOs: 10, 16 or 41-46, and the complementary strand as set forth in any one of SEQ ID NOs: 13 or 47, are on separate nucleic acid sequence molecules that form the double stranded synthetic miR-135 molecule.

According to some embodiments of the invention, the nucleic acid sequence of the miR-135b as set forth in any one of SEQ ID NOs: 10, 16 or 41-46, and the complementary strand as set forth in any one of SEQ ID NOs: 13 or 47, form a hairpin loop structure.

According to some embodiments of the invention, the nucleic acid sequence of the miR-135b as set forth in any one of SEQ ID NOs: 10, 16 or 41-46, and the complementary strand as set forth in any one of SEQ ID NOs: 13 or 47, are 100% complementary.

According to some embodiments of the invention, the cell-targeting moiety is conjugated to a 5′ end of the nucleic acid sequence of the complementary strand.

According to some embodiments of the invention, the cell-targeting moiety is conjugated to a 5′ end of the nucleic acid sequence of the miR-135b.

According to some embodiments of the invention, the cell-targeting moiety is conjugated to a 3′ end of the nucleic acid sequence of the complementary strand.

According to some embodiments of the invention, the cell-targeting moiety is conjugated to a 3′ end of the nucleic acid sequence of the miR-135b.

According to some embodiments of the invention, the synthetic miR-135 molecule and the cell-targeting moiety are connected by a linking group.

According to some embodiments of the invention, the linking group comprises a compound selected from the group consisting of phosphodiester, phosphoramidite, phosphorothioate, carbamate, methylphosphonate, guanidinium, sulfamate, sulfamide, formacetal, thioformacetal, sulfone, amide and mixtures thereof.

According to some embodiments of the invention, the linking group comprises a C10 linker.

According to some embodiments of the invention, the cell-targeting moiety binds specifically to a molecule expressed or presented on brain cells.

According to some embodiments of the invention, the cell-targeting moiety binds specifically to a neurotransmitter transporter.

According to some embodiments of the invention, the cell-targeting moiety is selected from the group consisting of a serotonin reuptake inhibitor (SRI), a selective serotonin reuptake inhibitor (SSRI), a serotonin-norepinephrine reuptake inhibitor (SNRI), a noradrenergic and specific serotoninergic antidepressant (NASSA), a noradrenaline reuptake inhibitor (NRI), a dopamine reuptake inhibitor (DRI), an endocannabinoid reuptake inhibitor (eCBRI), an adenosine reuptake inhibitor (AdoRI), an excitatory Amino Acid Reuptake Inhibitor (EAARI), a glutamate reuptake inhibitor (GluRI), a GABA Reuptake Inhibitor (GRI), a glycine Reuptake Inhibitor (GlyRI) and a Norepinephrine-Dopamine Reuptake Inhibitor (NDRI).

According to some embodiments of the invention, the selective serotonin reuptake inhibitor (SSRI) is selected from the group consisting of sertraline, a sertraline-structural analog, fluoxetine, fluvoxamine, paroxetine, indapline, zimelidine, citalopram, dapoxetine, escitalopram, and mixtures thereof.

According to some embodiments of the invention, when the cell-targeting moiety is sertraline the conjugate has the structure:

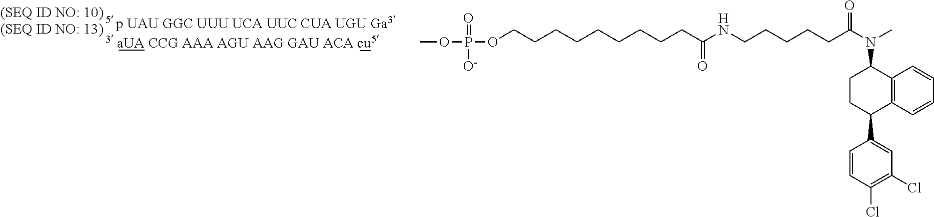 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to some embodiments of the invention, when the cell-targeting moiety is sertraline the conjugate has the structure:

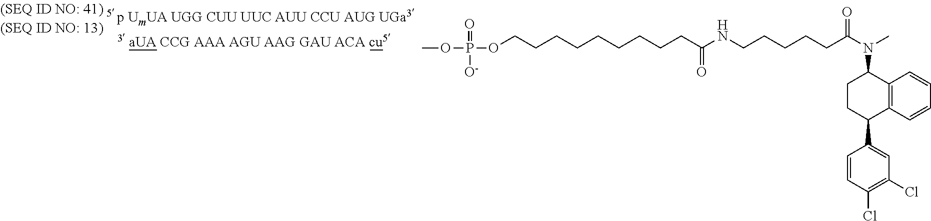 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to some embodiments of the invention, when the cell-targeting moiety is sertraline the conjugate has the structure:

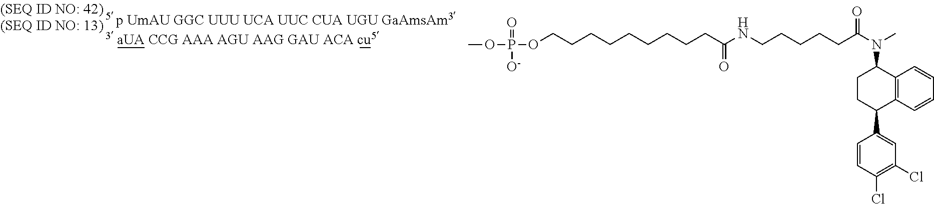 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to some embodiments of the invention, when the cell-targeting moiety is sertraline the conjugate has the structure:

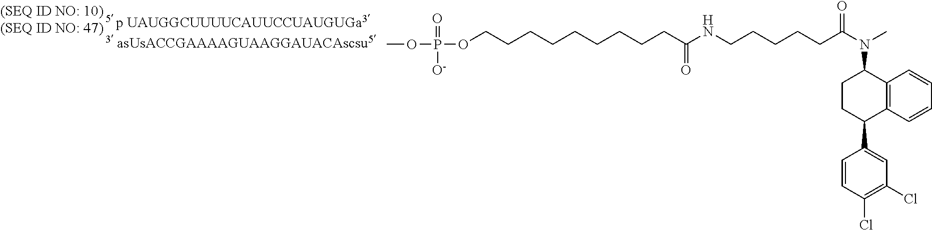 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to some embodiments of the invention, the cell-targeting moiety binds specifically to a tumor-associated antigen.

According to some embodiments of the invention, the cell-targeting moiety binds specifically to a molecule expressed or presented on bone cells, muscle cells or gastrointestinal cells.

According to some embodiments of the invention, the composition of matter or conjugate of some embodiments of the invention further comprises at least one of a cell-penetrating moiety or a moiety for transport across the blood brain barrier (BBB).

According to some embodiments of the invention, the composition of matter or conjugate of some embodiments of the invention further comprises a linker between the cell-penetrating moiety or the moiety for transport across the BBB and the synthetic miR-135 molecule.

According to some embodiments of the invention, the cell-penetrating moiety or the moiety for transport across the BBB is attached to the miR-135b and/or to the complementary strand and/or to the cell-targeting moiety.

According to some embodiments of the invention, the administering is effected by a mode of administration selected from the group consisting of an intranasal, an intracerebroventricular, an intrathecal, an oral, a local injection, and an intravenous mode of administration.

According to some embodiments of the invention, the composition is for intranasal, intracerebroventricular, intrathecal, oral, local injection, or intravenous administration.

According to some embodiments of the invention, the CNS-related condition is a psychiatric disorder.

According to some embodiments of the invention, the CNS-related condition is selected from the group consisting of a depression, an anxiety, an autism spectrum disorder, a schizophrenia, a bipolar disease, a stress, a fatigue, an impaired cognitive function, a panic attack, a compulsive behavior, an addiction, a social phobia, a sleep disorder, an eating disorder, a memory disorder, a cognition disorder, a growth disorder and a reproduction disorder.

According to some embodiments of the invention, the depression-related disorder is selected from the group consisting of a major depression, an obsessive-compulsive disorder (OCD), a pervasive developmental disorder (PDD), a post-traumatic stress disorder (PTSD), an anxiety disorder, a bipolar disorder, an eating disorder and a chronic pain.

According to some embodiments of the invention, the cancerous disease is selected from the group consisting of ovarian cancer, colorectal cancer, and prostate cancer.

According to some embodiments of the invention, the subject has a bone-related disease or condition selected from the group consisting of osteoporosis, bone fracture or deficiency, primary or secondary hyperparathyroidism, osteoarthritis, periodontal disease or defect, an osteolytic bone disease, post-plastic surgery, post-orthopedic implantation, post-dental implantation.

According to some embodiments of the invention, the subject has a muscle related disease or condition selected from the group consisting of a muscle degeneration disease, a neuromuscular disease, a spinal muscular atrophy, an inflammatory muscle disease and a metabolic muscle disease.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 depicts synthesis of miR-135 single strand oligonucleotides. The synthesis was carried out employing a typical experimental procedure of solid-phase synthesis on a CPG (Controlled Pore Glass) support. A typical oligonucleotide synthesis preceded through a series of cycles composed of fours steps (deprotection, coupling, capping and oxidation) that were repeated until the 5′ most nucleotide was attached (as described in detail in the ‘General materials and experimental procedures’ section below).

FIG. 2 illustrates synthesis of sertraline-conjugated miR-135. The addition of Sertraline and linkers to the 5′ end was carried out as follows:
1. Carboxy-C10 Linker addition: After the last cycle (DMT-OFF), a carboxy-C10 linker amidite block (in its N-hydroxysuccinimide ester form) was attached to the sequence in the final coupling step to facilitate conjugation of derivatized Sertraline (Sertraline bound to a C6-NH₂) onto the 5′ end of the oligonucleotide.
2. Sertraline addition: derivatized Sertraline was conjugated to 5′-carboxy-C10 modified oligonucleotide through and amide bond. This condensation was carried out under organic conditions (Diisopropylethylamine and Dimethylformamide (DIPEA/DMF), in room temperature for 24 hours, as discussed in detail in the Examples section below).

FIGS. 3A-B depict Luciferase reporter assay results demonstrating that Duplex 11 significantly targets Slc6a4 3′UTR (FIG. 3A) and HTR1a 3′UTR (FIG. 3B).

FIGS. 4A-E depict the effects of miR-135 mimetics on serotonergic function in-vivo. Of note, systemic 8-OH-DPAT administration (1 mg/kg body weight (BW), i.p.) did not evoke hypothermia in naked (i.e. unconjugated) miR-135-treated (100 μg) mice 24 (FIG. 4A), 48 (FIG. 4B), 72 (FIG. 4C) and 96 (FIG. 4D) hours following treatment. Two-way analysis of variance showed a significant effect ***P<0.001 versus control groups (n=5-10). No difference was found in the basal temperature between the groups (FIG. 4E).

FIGS. 5A-D illustrate the effect of sertraline-conjugated miR-135 mimetic Duplex 11 (miCure-135-1, as set forth in SEQ ID Nos: 10 and 13) on serotonergic function following direct dorsal raphe nucleus (DRN) administration. Of note, systemic 8-OH-DPAT administration (1 mg/kg BW, i.p.) did not evoke hypothermia in sertraline-conjugated miR-135-treated (100 μg) mice, 24 hours (FIG. 5A), 48 hours (FIG. 5B), 96 hours (FIG. 5C) and 7 days (FIG. 5D) following treatment. Two-way analysis of variance showed a significant effect ***P<0.001 versus control groups (n=10).

FIGS. 6A-H illustrate that acute administration of sertraline-conjugated miR-135 (miCure-135-1) at a lower dose (30 μg) silences 5HT1a and SERT and evokes anti-depressant-like responses. Of note, systemic 8-OH-DPAT administration (1 mg/kg BW, i.p.) did not evoke hypothermia in sertraline conjugated miR-135-treated (30 μg) mice one day (FIG. 6A), two days (FIG. 6B), four days (FIG. 6C) and seven days (FIG. 6D) following treatment. Two-way analysis of variance showed a significant effect ***P<0.001 versus control groups (n=10). No difference was found in the basal temperature between the groups (FIG. 6E) (n=10-40). Increased extracellular serotonin was uncovered in medial prefrontal cortex (mPFC) of miCure-135-1 treated mice during the tail suspension test. Significant effect *P<0.05 versus controls (FIG. 6F). Single intracerebral miCure-135-1 administration (30 μg) evoked a decreased immobility in the tail suspension test (n=8-9) (FIG. 6G). Autoradiography of [3H]-citalopram binding showed a reduction of SERT density in the dorsal raphe of treated mice compared to control (n=5-7) *p<0.05 (FIG. 6H).

FIGS. 7A-C illustrate that acute intranasal administration of miCure-135-1 (166 μg) silences 5HT1a and evokes anti-depressant-like responses. Of note, systemic 8-OH-DPAT administration (1 mg/kg BW, i.p.) did not evoke hypothermia following intranasal delivery of sertraline conjugated miR-135 treated (166 μg) mice 5 days following treatment. Two-way analysis of variance showed a significant effect groups (n=5) *P<0.05 versus control (FIG. 7A). Single intranasal miCure-135-1 administration (166 μg) evoked a decreased immobility in the tail suspension test 4 days following treatment (n=8-9) *P<0.05 versus control (FIG. 7B). Mice that received a single intranasal miCure-135-1 administration (166 μg) explored the light compartment longer than control in the dark/light transfer test (n=11-12) *P<0.05 versus control (FIG. 7C).

FIG. 8 depicts the additional miR-135 modifications carried out. The modifications are as follows:
High case letters (e.g. A, U, C, G): RNA
Lower-case letters (e.g. a, u, c, g): 2′-O-Me modification
Um: (2′-O-MOE-5′-Me) Uracil modification
Am: (2′-O-MOE) Adenine modification
Lower-case ‘s’: phosphorothioate. No indication means a normal phosphodiester bond.
P: phosphate
Underscore: 2′-fluoro, i.e. 2′-F

FIGS. 9A-B depict results of Luciferase reporter assay demonstrating that miCure-135-1, miCure-135-2, miCure-135-3, miCure-135-9, miCure-135-10 and miCure-135-11 significantly target Slc6a4 3′UTR (FIG. 9B) and HTR1a 3′UTR (FIG. 9A). The numbers above the bars indicate the number of significant statistical differences found (out of 5 experiments).

FIGS. 10A-E depict the immune response of human peripheral blood mononuclear cells (PBMCs) following treatment with 3 different miR-135 conjugated duplexes in-vitro. Of note, miCure-135-10 induced high secretion of TNF-alpha cytokine especially at the concentration of 300 nM, while miCure-135-1 moderately activated this cytokine and miCure-135-2 induced almost no secretion (FIG. 10A). The same pattern of secretion was demonstrated for IFN-alpha-2a cytokine (FIG. 10B), where miCure-135-10 induced a high level of secretion, miCure-135-1 induced moderate levels of secretion and miCure-135-2 no secretion. None of the tested duplexes induced secretion of IL-10 (FIG. 10C), IL-1beta (FIG. 10D) nor IL-6 (FIG. 10E). In FIGS. 10A, 10C-E: dir. incubation refers to direct incubation.

FIGS. 11A-F depict the immune response of human peripheral blood mononuclear cells (PBMCs) following treatment with an additional set of 3 miR-135 conjugated duplexes in-vitro. Of note, miR-135-11 and miCure-135-9 induced high secretion of IFN-alpha-2a while miCure-135-3 induced almost no secretion thereof (FIG. 11A). A low level of TNF-alpha secretion was induced by miCure-135-11 and miCure-135-9 where no secretion was induced by miCure-135-3 (FIG. 11B). miCure-135-9 at a 300 nM concentration showed a low level of secretion of IFN-gamma, whereas miCure-135-3 and miCure-135-9 did not show secretion thereof (FIG. 11C). Neither of the 3 tested duplexes lead to a noticeable production of cytokine IL-10 (FIG. 11D), IL-1b (FIG. 11E), nor IL-6 (FIG. 11F).

FIGS. 12A-G depict the effect of the sertraline conjugated miR-135 mimetics (miCure-135-2, miCure-135-3 and miCure-135-9) on serotonergic function and their anti-depressant-like effect. Systemic 8-OH-DPAT administration (1 mg/kg BW, i.p.) did not evoke hypothermia in miCure-135-2-treated (30 μg) mice two days (FIG. 12A), four days (FIG. 12B) and seven days (FIG. 12C) days following treatment. Two-way analysis of variance showed a significant effect * P<0.05, ***P<0.001 versus control groups (n=4-5). Systemic 8-OH-DPAT administration (1 mg/kg BW, i.p.) did not evoke hypothermia in miCure-135-9-treated (30 μg) mice two days following treatment (FIG. 12D). Two-way analysis of variance showed a significant effect *P<0.05 versus control groups (n=4). No difference was found in the basal temperature between the groups (FIG. 12E) (n=4-19). Local selective serotonin reuptake inhibitor (Citalopram 10 μM) infusion by reverse-dialysis lead to an increase of extracellular 5-hydroxytryptamine (5-HT) in the PFC of sertraline conjugated control (100 μg) treated mice as compared to miCure-135-3 (100 μg) treated mice (FIG. 12F). Two-way analysis of variance showed a significant effect *P<0.05 versus control groups (n=6). Single intracerebral miCure-135-3 administration (100 μg) evoked a decreased immobility in the tail suspension test (n=6) *p<0.05 (FIG. 12G).

FIGS. 13A-F depict that intranasal and intracerebroventricular (ICV) administration of sertraline conjugated miR-135 mimetics maintain the effect on serotonergic function in-vivo. Of note, systemic 8-OH-DPAT administration (1 mg/kg BW, i.p.) did not evoke hypothermia in mice treated with miCure-135-3 (50 μg) (FIG. 13A), (100 μg) (FIG. 13B) and (200 μg) (FIG. 13C) 48 hours following treatment. The same effect was illustrated following 7 days of intranasal administration of miCure-135-2 (33 μg/day) 24 hours following treatment (FIG. 13D) and following 7 days of ICV administration of miCure-135-3 (200 μg/day) 96 hours following treatment (FIG. 13E). Two-way analysis of variance showed a significant effect *P<0.05 versus control groups (n=6). Mice treated with intranasal administration for 7 days of miCure-135-3 (200 μg/day) had a smaller increase of extracellular 5-hydroxytryptamine (5-HT) in the PFC following local selective serotonin reuptake inhibitor (Citalopram) infusion by reverse-dialysis compare to sertraline conjugated control (200 μg/day) treated mice (FIG. 13F). Two-way analysis of variance showed a significant effect *P<0.05 versus control groups (n=6).

FIGS. 14A-G illustrate that acute intranasal administration of miCure-135-3 (2500 μg) effects the serotonergic function and evokes anti-depressant-like responses. Immunoblot image and quantification demonstrate the reduction in the protein levels of SERT (FIGS. 14A-B) and HTR1AR (FIGS. 14C-D) in the dorsal raphe of treated mice compared to control (n=6-7) *p<0.05; Reduced extracellular serotonin in mPFC of controls, but not in treated mice after 8-OH-DPAT administration (1 mg kg⁻¹, i.p.) (n=5-6), significant effect of group (P<0.05) versus controls (FIG. 14E); Local selective serotonin reuptake inhibitor (Citalopram 10 μM) infusion by reverse-dialysis led to an increase of extracellular 5-hydroxytryptamine (5-HT) in the PFC of sertraline conjugated control (2500 μg) treated mice as compared to miCure-135-3 (2500 μg) treated mice (FIG. 14F); Two-way analysis of variance showed a P value of 0.05 versus control groups (n=6). Single intranasal miCure-135-3 administration (2500 μg) evoked a decreased immobility in the tail suspension test (n=8-14) *p<0.05 (FIG. 14G).

FIG. 15 illustrates that acute intranasal administration of miCure-135-3 (2500 μg) evokes anti-depressant-like responses in mice that underwent induction of depression-like behavior. Mice underwent a 28 day protocol that induces a depression-like behavior (see GENERAL MATERIALS AND EXPERIMENTAL PROCEDURES section, below) followed by a single intranasal administration of control or miCure-135-3. The results demonstrate that a single intranasal miCure-135-3 administration (2500 μg) evoked a decreased immobility in the tail suspension test 3 days following treatment (n=8-10) *P<0.05 versus control.

FIGS. 16A-D illustrate the localization of miCure-135-3 in the dorsal raphe nucleus following intranasal administration. Selective accumulation of alexa488-labeled miCure-135-3 (green) in tryptophan hydroxylase2-positive (TPH2-positive) neurons following intranasal administration (1000 μg). Confocal images show co-localization of alexa488-labeled miCure-135-3 (yellow) in dorsal raphe nucleus (DR) 5-HT neurons (TPH2-positive, red). Cell nuclei were stained with DAPI (4,6-diamidino-2-phenylindole; blue). Each row represents a different mouse (n=3, FIG. 16A); High-magnification photomicrographs of the frames depicted in ‘exm1’ of FIG. 16A were provided to show the co-localization of alexa488-labeled miCure-135-3 in dorsal raphe nucleus (DR) 5-HT neurons (FIGS. 16B-C). Scale bars: left=100 μm, right=25 μm. In contrast, alexa488-labeled miCure-135-3 was absent in cells of brain areas close to the application site (olfactory bulbs) or to brain ventricles (hippocampus and striatum) (FIG. 16D).

FIGS. 17A-D illustrate the immunohistochemical assessment of cellular viability in the raphe nuclei after acute administration of miCure-135-3 (100 μg). Conjugate-control, miCure-135-3 or a positive control that was reported previously to have no effect on cellular viability were acutely delivered directly into the dorsal raphe nucleus. Adjacent 30-km-thick sections through the midbrain raphe nuclei were stained with neuronal NeuN (FIG. 17A), microglial Iba1 (FIG. 17B), or serotonergic TPH (FIG. 17C) markers. Representative images of midbrain raphe nuclei stained with NeuN, Iba1 and TPH under the different treatments are represented in FIG. 17D. Of note, no differences were found between all experimental groups for any of the markers. Representative pictures are depicted in FIG. 17C, Scale bar: 100 km.

### DESCRIPTION OF SPECIFIC EMBODIMENTS OF THE INVENTION

The present invention, in some embodiments thereof, relates to therapeutic miR-135 molecules and, more particularly, but not exclusively, to the use of same.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

MicroRNAs (miRs) are a subset of small RNA molecules that regulate gene expression post-transcriptionally and are abundant in various tissues including the brain.

While reducing the present invention to practice, the present inventors have synthesized a novel synthetic miR-135 molecule for therapeutics. Furthermore, the present inventors have uncovered that it is possible to target a miR-135 molecule to a cell of interest by coupling the miR-135 molecule to another molecule which is capable of specifically binding the cell of interest.

Specifically, the present inventors designed and synthesized miR-135 mimetic molecules comprising two strands, i.e. a guide strand and a passenger strand, based on endogenous miR-135 with few modifications aimed to improve stability and cell penetration (see Tables 1 and 6, herein below, and FIG. 8). The miR-135 mimetic molecules (termed duplexes 1-17 in Table 1 and duplexes 1-16 in Table 6) were then screened in vitro on known miR-135 targets (e.g. Htr1a and Slc6a4). One of the synthesized mimetic miR-135 molecules (termed duplex 11 in Table 1 and duplex 1 in Table 6, as set forth in SEQ ID Nos: 10 and 13) was shown to bind miR-135 targets (e.g. Htr1a and Slc6a4) significantly better than the other molecules synthesized and tested in the human cell line HEK293T (see FIGS. 3A-B and Example 1 herein below). This miR-135 mimetic molecule (i.e. duplex 11 in Table 1 and duplex 1 in Table 6) comprised the guide sequence of miR-135b (as set forth in SEQ ID NO: 37) and a complementary sequence which is distinct from the native sequence (as set forth in SEQ ID NO: 40). Moreover, this synthetic miR-135 molecule (i.e. duplex 11 in Table 1 and duplex 1 in Table 6) was shown to comprise serotonergic function in vivo in animal models (see FIGS. 4A-E and Example 2 herein below).

The present inventors further designed and synthesized sertraline-conjugated miR-135 molecules for non-invasive delivery to the brain. In vivo studies with the sertraline-conjugated miR-135 mimetic Duplex 11 (termed herein miCure-135-1) illustrated that a single administration into the dorsal raphe nucleus (DRN) was sufficient to abolish hypothermia induced by the selective HTR1a agonist 8-OH-DPAT for up to 7 days in naïve mice (FIGS. 5A-D and 6A-E, and Examples 3-4 herein below). Administration of a sertraline-conjugated miR-135 molecule into the DRN was further shown to induce an anti-depressive effect as well as a better coping mechanism in treated mice (FIGS. 6F-G and Example 4 herein below). Administration of the sertraline-conjugated miR-135 molecule into the DRN was also shown to silence the SERT gene (FIG. 6H and Example 4 herein below). Moreover, non-invasive, intranasal administration of sertraline-conjugated miR-135 silenced 5HT1a, as illustrated by the lower hypothermic response (FIG. 7A and Example 5 herein below), and evoked an anti-depressant/anxiolytic-like response in treated mice (FIGS. 7B-C and Example 5 herein below).

In further experiments, the present inventors uncovered that additional sertraline conjugated miR-135 mimetics comprising various chemical modifications, namely, miCure-135-1, miCure-135-2, miCure-135-3, miCure-135-9, miCure-135-10 and miCure-135-11 (see Table 6, herein below), are potent and significantly modify both 5HTR1A and SLC6a4 levels (using 3′UTR luciferase constructs, FIGS. 9A-B and Example 6 herein below). The effect of the conjugated miR-135 mimetics on immune activation was tested. As evident from the results (see FIGS. 10A-E and FIGS. 11A-F, and Example 7, herein below), the different conjugated miR-135 mimetics induced a varied immune response with miCure-135-1, miCure-135-2 and miCure-135-3 inducing the lowest cytokine secretion from PBMCs. Moreover, acute administration of the sertraline conjugated miR-135 mimetics miCure-135-2, miCure-135-3 and miCure-135-9 into the dorsal raphe nucleus (DRN) affected serotonergic function and evoked an anti-depressant-like response (see FIGS. 12A-G, and Example 8, herein below). Furthermore, intranasal and intracerebroventricular administration of sertraline-conjugated miR-135 mimetics successfully reduced the serotonergic auto receptor (5HT1a) and the serotonin transporter (SERT) in the dorsal raphe nucleus (see FIGS. 13A-F and Example 9, herein below).

In further experiments, the present inventors illustrated that acute intranasal administration of themiR-135 mimetic miCure-135-3 reduced the protein levels of SERT and 5-HT1A-auto receptor (HTR1AR) and had an anti-depressant affect (Examples 10 and 11, herein below). Intranasal administration of miCure-135-3 resulted in accumulation in the dorsal raphe nucleus, and specifically in the midbrain 5-HT neurons (Example 12, herein below). Furthermore, intranasal administration of the miR-135 mimetic miCure-135-3 was found to be safe for in vivo administration (Example 13, herein below).

Accordingly, the synthetic miR-135 molecules of the invention as well as the conjugated form thereof may be used for therapeutics such as for treatment of CNS-related conditions including psychiatric disorders.

Thus, according to one aspect of the present invention there is provided a composition of matter comprising a synthetic miR-135 molecule comprising a nucleic acid sequence of a miR-135b as set forth in SEQ ID NO: 37, and a complementary strand as set forth in SEQ ID NO: 40.

According to one aspect of the present invention there is provided a synthetic miR-135 molecule comprising a nucleic acid sequence of a miR-135b as set forth in SEQ ID NO: 37, and a complementary strand as set forth in SEQ ID NO: 40.

As used herein “synthetic” refers to a non-naturally molecule.

According to one embodiment, the non-naturally occurring miR-135 comprises the sequence of the naturally occurring mature miR-135b and comprises a synthetic backbone and/or side chain. Typically, the synthetic miR-135 molecule functions in a cell or under physiological conditions as a naturally occurring miRNA, e.g. miR-135b.

As used herein, the term “miR-135b” refers to the microRNA molecule that is involved in post-transcriptional gene regulation and includes miR-135b 5 prime (i.e. miR-135b, also referred to as miR-135b-5p) or 3 prime (i.e. miR-135b*, also referred to as miR-135b-3p). An exemplary mature miR-135b includes, but is not limited to, miR-135b as set forth in Accession No. MIMAT0000758 (SEQ ID NO: 37). An exemplary mature miR-135b* includes, but is not limited to, miR-135b* as set forth in Accession No. MIMAT0004698 (SEQ ID NO: 38).

MicroRNAs are typically processed from pre-miR (pre-microRNA precursors). Pre-miRs are a set of precursor miRNA molecules transcribed by RNA polymerase III that are efficiently processed into functional miRNAs, e.g., upon transfection into cultured cells. A pre-miR can be used to elicit specific miRNA activity in cell types that do not normally express this miRNA, thus addressing the function of its target by down regulating its expression in a “gain of (miRNA) function” experiment. Pre-miR designs exist to all of the known miRNAs listed in the miRNA Registry and can be readily designed for any research. The microRNAs may be administered to the cell per se or ligated into a nucleic acid construct, as further described herein below.

It will be appreciated that the microRNAs of the present teachings (e.g. miR-135) may bind, attach, regulate, process, interfere, augment, stabilize and/or destabilize any microRNA direct or indirect target (e.g. miR-135 target). Such a target can be any molecule, including, but not limited to, DNA molecules, RNA molecules and polypeptides, such as but not limited to, serotonin related genes, such as the serotonin transporter (i.e. SERT or Slc6a4), the serotonin inhibitory receptor 1a (Htr1a), tryptophan hydroxylase 2 (Tph2) and monoamine hydroxylase (MaoA); adrenaline or noradrenaline receptors (adrenergic receptors such as Adr1); Adenylate cyclase type 1 (ADCY1); CRH receptors such as Crh1R; or any other molecules e.g. FK506 binding protein 5 (FKBP5), Translin-associated protein X (Tsnax) and Cell adhesion molecule L1 (L1cam); as well as other targets associated with psychiatric disorders including those listed in Table 7, herein below.

Additional direct or indirect targets include, but are not limited to, adenylate cyclase activating polypeptide 1 (Adcyap1 or PACAP); adenylate cyclase activating polypeptide 1 receptor 1 (Adcyap1r1); adrenergic receptor, alpha 2a (Adra2a); an ankyrin 3 (ANK3); activity-regulated cytoskeleton-associated protein (Arc); Rho GTPase activating protein 6 (Arhgap6); activating transcription factor 3 (Atf3); beta-site APP cleaving enzyme 1 (Bace1); calcium channel, voltage-dependent, L type, alpha 1D subunit (Cacna1d); cell adhesion molecule 3 (Cadm3); complexin 1 (Cplx1); complexin 2 (Cplx2); CUB and Sushi multiple domains 1 (Csmd1); casein kinase 1, gamma 1 (Csnk1g1); doublecortin (Dcx); DIRAS family, GTP-binding RAS-like 2 (Diras2); discs, large homolog 2 (Drosophila) (Dlg2); ELK1, member of ETS oncogene family (Elk1); fyn-related kinase (Frk); fucosyltransferase 9 (alpha (1,3) fucosyltransferase) (Fut9); gamma-aminobutyric acid (GABA-A) receptor, subunit beta 2 (Gabrb2); GATA binding protein 3 (Gata3); growth hormone secretagogue receptor (Ghsr); G protein-coupled receptor 3 (Gpr3); a glutamate receptor, ionotropic AMPA3 (alpha 3) (GRIA3); glutamate receptor, ionotropic, kainate 3 (Grik3); G protein-coupled receptor kinase 5 (Grk5); a glycogen synthase kinase-3beta (GSK3B); hyperpolarization activated cyclic nucleotide-gated potassium channel 1 (Hcn1), hyperpolarization-activated, cyclic nucleotide-gated K+2 (Hcn2), 5-hydroxytryptamine (serotonin) receptor 1A (Htr1a); inositol monophosphatase (IMPA1), kalirin, RhoGEF kinase (Kalrn); a potassium intermediate/small conductance calcium-activated channel, subfamily N, member 3 (KCNN3); karyopherin alpha 3 (importin alpha 4) (Kpna3); myelin transcription factor 1-like (Mytl1); nuclear receptor coactivator 2 (Ncoa2); N-Myc Downstream-Regulated Gene 4 (Ndrg4); a nitric oxide synthase 1 (neuronal) adaptor protein (NOS1AP); nuclear receptor subfamily 3, group C, member 2 (Nr3c2); netrin G1 (Ntng1); nuclear casein kinase and cyclin-dependent kinase substrate 1 (Nucks1); phosphodiesterase 1A, calmodulin-dependent (Pde1a); phosphodiesterase 4A, cAMP specific (Pde4a); phosphodiesterase 8B (Pde8b); phospholipase C, beta 1 (Plcb1); prolactin receptor (Pr1r); RAB1B, member RAS oncogene family (Rab1b); Ras-Related Protein Rap-2a (Rap2a); Retinoid-Related Orphan Receptor Beta (Rorb); sirtuin 1 (silent mating type information regulation 2, homolog) 1 (Sirt1); solute carrier family 12, (potassium/chloride transporters) member 6 (Slc12a6); solute carrier family 5 (choline transporter), member 7 (Slc5a7); solute carrier family 6 (neurotransmitter transporter, serotonin), member 4 (Slc6a4); trans-acting transcription factor 1 (Sp1); synaptic vesicle glycoprotein 2 b (Sv2b); Synaptic nuclear envelope 1 (encodes nesprin-1) (Syne1); synaptotagmin I (Syt1); synaptotagmin II (Syt2); synaptotagmin III (Syt3); transforming growth factor, beta receptor II (Tgfbr2); thyroid hormone receptor, beta (Thrb); transient receptor potential cation channel, subfamily C, member 6 (Trpc6); vesicle-associated membrane protein 2 (Vamp2); wingless-related MMTV integration site 3 (Wnt3); and zinc finger, BED domain containing 4 (Zbed4).

TABLE 7
Putative targets of miR-135 associated with psychiatric disorders

| Human | |
|---|---|
| ortholog | Gene name |
| Adcyap1 | adenylate cyclase activating polypeptide 1 (also PACAP) |
| Adcyap1r1 | adenylate cyclase activating polypeptide 1 receptor 1 |
| Adra2a | adrenergic receptor, alpha 2a |
| Ank3 | ankyrin 3, epithelial |
| Arc | activity-regulated cytoskeleton-associated protein |
| Arhgap6 | Rho GTPase activating protein 6 |
| Atf3 | activating transcription factor 3 |
| Bace1 | beta-site APP cleaving enzyme 1 |
| Cacna1d | calcium channel, voltage-dependent, L type, alpha 1D subunit |
| Cadm3 | cell adhesion molecule 3 |
| Cplx1 | complexin 1 |
| Cplx2 | complexin 2 |
| Csmd1 | CUB and Sushi multiple domains 1 |
| Csnk1g1 | casein kinase 1, gamma 1 |
| Dcx | doublecortin |
| Diras2 | DIRAS family, GTP-binding RAS-like 2 |
| Dlg2 | discs, large homolog 2 (Drosophila) |
| Elk1 | ELK1, member of ETS oncogene family |
| Frk | fyn-related kinase |
| Fut9 | fucosyltransferase 9 (alpha (1,3) fucosyltransferase) |
| Gabrb2 | gamma-aminobutyric acid (GABA-A) receptor, subunit beta 2 |
| Gata3 | GATA binding protein 3 |
| Ghsr | growth hormone secretagogue receptor |
| Gpr3 | G protein-coupled receptor 3 |
| Gria3 | glutamate receptor, ionotropic, AMPA3 (alpha 3) |
| Grik3 | glutamate receptor, ionotropic, kainate 3 |
| Grk5 | G protein-coupled receptor kinase 5 |
| Gsk3b | glycogen synthase kinase 3 beta |
| Hcn1 | hyperpolarization activated cyclic nucleotide-gated potassium |
| | channel 1 |
| Hcn2 | hyperpolarization-activated, cyclic nucleotide-gated K+ 2 |
| Htr1a | 5-hydroxytryptamine (serotonin) receptor 1A |
| Impa1 | inositol (myo)-1(or 4)-monophosphatase 1 |
| Kalrn | kalirin, RhoGEF kinase |
| Kcnn3 | potassium intermediate/small conductance calcium-activated channel, |
| | subfamily N, member 3 |
| Kpna3 | karyopherin alpha 3 (importin alpha 4) |
| Myt1l | myelin transcription factor 1-like |
| Ncoa2 | nuclear receptor coactivator 2 |
| Ndrg4 | N-Myc Downstream-Regulated Gene 4 |
| Nos1ap | nitric oxide synthase 1 (neuronal) adaptor protein |
| Nr3c2 | nuclear receptor subfamily 3, group C, member 2 |
| Ntng1 | netrin G1 |
| Nucks1 | nuclear casein kinase and cyclin-dependent kinase substrate 1 |
| Pde1a | phosphodiesterase 1A, calmodulin-dependent |
| Pde4a | phosphodiesterase 4A, cAMP specific |
| Pde8b | phosphodiesterase 8B |
| Plcb1 | phospholipase C, beta 1 |
| Prlr | prolactin receptor |
| Rab1b | RAB1B, member RAS oncogene family |
| Rap2a | Ras-Related Protein Rap-2a |
| Rorb | Retinoid-Related Orphan Receptor Beta |
| Sirt1 | sirtuin 1 (silent mating type information regulation 2, homolog) 1 |
| Slc12a6 | solute carrier family 12, (potassium/chloride transporters) |
| | member 6 |
| Slc5a7 | solute carrier family 5 (choline transporter), member 7 |
| Slc6a4 | solute carrier family 6 (neurotransmitter transporter, serotonin), |
| | member 4 |
| Sp1 | trans-acting transcription factor 1 |
| Sv2b | synaptic vesicle glycoprotein 2 b |
| Syne1 | Synaptic nuclear envelope 1 (encodes nesprin-1) |
| Syt1 | synaptotagmin I |
| Syt2 | synaptotagmin II |
| Syt3 | synaptotagmin III |
| Tgfbr2 | transforming growth factor, beta receptor II |
| Thrb | thyroid hormone receptor, beta |
| Trpc6 | transient receptor potential cation channel, subfamily C, member 6 |
| Vamp2 | vesicle-associated membrane protein 2 |
| Wnt3 | wingless-related MMTV integration site 3 |
| Zbed4 | zinc finger, BED domain containing 4 |

A “nucleic acid” as used herein generally refers to a molecule (single-stranded or double-stranded oligomer or polymer) of RNA or a derivative, mimic or analog thereof, comprising a nucleobase. A nucleobase includes, for example, a naturally occurring purine or pyrimidine base found in RNA (e.g., an adenine “A,” a guanine “G,” an uracil “U” or a cytosine “C”). The term “nucleic acid” encompasses the terms “oligonucleotide” and “polynucleotide,” each as a subgenus of the term “nucleic acid.” The term “nucleic acid” further includes nucleic acids derived from synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to respective naturally occurring portions. Such modified or substituted oligonucleotides may be preferred over native forms because of desirable properties such as, for example, enhanced cellular uptake, enhanced affinity for nucleic acid target and increased stability in the presence of nucleases, as discussed in further detail below.

The terms “mimic” or “analog” refer to a molecule that may or may not structurally resemble a naturally occurring molecule, but possesses similar functions.

The synthetic miR-135 molecule of some embodiments of the invention is a double stranded nucleic acid molecule comprising a miR-135b and a complementary strand. Such a double stranded nucleic acid molecule is similar in structure to the naturally occurring miRNA precursor and can be bound and processed by the cellular protein complex into the active mature miRNA.

According to a specific embodiment, the miR-135b (also referred to as “guide strand” or “active strand”) comprises a sequence having at least 90%, 91%, 92%, 93%, 94%, 95%, 96%, 97%, 98%, 99% or 100% sequence homology or identity to the endogenous mature miR-135b. According to a specific embodiment, the miR-135b comprises a sequence that is comparable (e.g. identical) to the endogenous mature miR-135b.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire nucleic acid sequences of the invention and not over portions thereof.

The degree of homology or identity between two or more sequences can be determined using various known sequence comparison tools. Following is a non-limiting description of such tools which can be used along with some embodiments of the invention.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used.

According to some embodiment, determination of the degree of homology further requires employing the Smith-Waterman algorithm (for protein-protein comparison or nucleotide-nucleotide comparison).

According to some embodiments of the invention, the global homology is performed on sequences which are pre-selected by local homology to the polypeptide or polynucleotide of interest (e.g., 60% identity over 60% of the sequence length), prior to performing the global homology to the polypeptide or polynucleotide of interest (e.g., 80% global homology on the entire sequence). For example, homologous sequences are selected using the BLAST software with the Blastp and tBlastn algorithms as filters for the first stage, and the needle (EMBOSS package) or Frame+ algorithm alignment for the second stage. Local identity (Blast alignments) is defined with a very permissive cutoff—60% Identity on a span of 60% of the sequences lengths because it is used only as a filter for the global alignment stage. In this specific embodiment (when the local identity is used), the default filtering of the Blast package is not utilized (by setting the parameter “−F F”). In the second stage, homologs are defined based on a global identity of at least 80% to the core gene polypeptide sequence.

According to a specific embodiment, the miR-135b comprises a sequence as set forth in SEQ ID NO: 37.

According to a specific embodiment, the miR-135b comprises a sequence as set forth in any one of SEQ ID NOs: 1, 5, 7, 8, 10, 14, 16, 41, 42, 43, 44, 45 or 46.

According to a specific embodiment, the complementary strand of the synthetic miR-135 molecule (also referred to herein as “passenger strand”) comprises a sequence having at least about 50%, 60%, 70%, 80%, 85%, 90%, 91%, 92%, 93%, 94%, 95%, 96%, 97%, 98%, 99% or 100% complementarity to the mature miR-135b sequence.

According to a specific embodiment, the complementary strand comprises a sequence that is 100% complementary, i.e. complete match, to the guide strand.

According to a specific embodiment, the complementary strand comprises a sequence as set forth in SEQ ID NO: 40.

According to a specific embodiment, the complementary strand comprises a sequence as set forth in SEQ ID NO: 2, 3, 4, 6, 9, 11, 12, 13, 15 or 47.

According to a specific embodiment, the nucleic acid sequence of miR-135b (e.g. as set forth in SEQ ID NO: 37) and the nucleic acid sequence of the complementary strand (e.g. as set forth in SEQ ID NO: 40) are 100% complementary over the entire nucleic acid sequences (e.g. over the entire length of SEQ ID NO: 37 and SEQ ID NO: 40).

According to a specific embodiment, there are no overhangs between the guide strand and the passenger strand of the synthetic miR-135 molecules of some embodiments of the invention.

According to one embodiment, the synthetic miR-135 molecule comprises a nucleic acid sequence of a miR-135b as set forth in SEQ ID NO: 37.

According to one embodiment, the synthetic miR-135 molecule comprises a complementary strand as set forth in SEQ ID NO: 40.

The length of the synthetic miR-135 molecule of the present invention is optionally of 100 nucleotides or less, optionally of 90 nucleotides or less, optionally 80 nucleotides or less, optionally 70 nucleotides or less, optionally 60 nucleotides or less, optionally 55 nucleotides or less, or optionally 50 nucleotides or less.

According to one embodiment, the synthetic miR-135 molecule comprises 46-50 residues.

According to one embodiment, the synthetic miR-135 molecule comprises 46-55 residues.

According to one embodiment, the synthetic miR-135 molecule comprises 46-60 residues.

According to one embodiment, the synthetic miR-135 molecule comprises 46-70 residues.

According to one embodiment, the synthetic miR-135 molecule comprises 46-80 residues.

According to a specific embodiment, the synthetic miR-135 molecule comprises at least about 46, 47, 48, 49, 50, 51, 52, 53, 54, 55, 56, 57, 58, 59, 60, 61, 62, 63, 64, 65, 66, 67, 68, 69 or 70 nucleic acid residues in length.

According to a specific embodiment, the synthetic miR-135 molecule comprises no more than 46, 47, 48, 49, 50, 51, 52, 53, 54, 55, 56, 57, 58, 59, 60, 61, 62, 63, 64, 65, 66, 67, 68, 69 or 70 nucleic acids in length.

According to a specific embodiment, the synthetic miR-135 molecule comprises no more than 52 nucleic acids.

According to a specific embodiment, the synthetic miR-135 molecule comprises no more than 50 nucleic acids.

According to a specific embodiment, the synthetic miR-135 molecule comprises no more than 48 nucleic acids.

According to a specific embodiment, the synthetic miR-135 molecule comprises 52 nucleic acids.

According to a specific embodiment, the synthetic miR-135 molecule comprises 50 nucleic acids.

According to a specific embodiment, the synthetic miR-135 molecule comprises 48 nucleic acids.

According to a specific embodiment, the synthetic miR-135 molecule comprises 46 nucleic acids.

According to one embodiment, the synthetic miR-135 molecule does not comprise a linker region. In such a case, the miR-135b region and the complementary region (i.e. the guide and passenger strands, respectively) are independent and are annealed as a duplex.

According to one embodiment, the nucleic acid sequence of miR-135b as set forth in SEQ ID NO: 37 and the complementary strand as set forth in SEQ ID NO: 40 are on separate nucleic acid sequence molecules that form the double stranded synthetic miR-135 molecule.

According to one embodiment, the nucleic acid sequence of miR-135b as set forth in any one of SEQ ID NOs: 1, 5, 7, 8, 10, 14, 16, 41, 42, 43, 44, 45 or 46 and the complementary strand as set forth in any one of SEQ ID NOs: 2, 3, 4, 6, 9, 11, 12, 13, 15 or 47 are on separate nucleic acid sequence molecules that form the double stranded synthetic miR-135 molecule.

According to one embodiment, the nucleic acid sequence of miR-135b as set forth in SEQ ID NO: 37 and the complementary strand as set forth in SEQ ID NO: 40 form a hairpin loop structure.

According to one embodiment, the nucleic acid sequence of miR-135b as set forth in any one of SEQ ID NOs: 1, 5, 7, 8, 10, 14, 16, 41, 42, 43, 44, 45 or 46 and the complementary strand as set forth in any one of SEQ ID NOs: 2, 3, 4, 6, 9, 11, 12, 13, 15 or 47 form a hairpin loop structure.

According to one embodiment, the synthetic miR-135 molecule comprises a linker region between the nucleic acid sequence of miR-135b (e.g. guide strand) and the nucleic acid sequence of the complementary sequence (e.g. passenger strand). Such a linker region may create a hairpin loop. Accordingly, the synthetic miR-135 molecule of some embodiments of the invention is capable of forming a hairpin loop structure as a result of bonding between the miR-135b region and the complementary region of the molecule.

According to one embodiment, the linker region comprises between 2 and 30 residues.

According to one embodiment, the linker region comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 residues in length.

According to one embodiment, the linker region comprises no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 residues in length.

According to one embodiment, the synthetic miR-135 molecule may comprise flanking sequences at either the 5′ or 3′ end of the miR-135b (e.g. guide strand) and/or the complementary sequence (e.g. passenger strand).

According to one embodiment, there are at least about 1, 2, 3, 4, 5, 6, 7, 8, 9, 10 nucleotides flanking one or both sides of the miR-135b (e.g. guide strand) and/or the complementary sequence (e.g. passenger strand).

According to one embodiment, the miR-135b (e.g. guide strand) and/or the complementary sequence (e.g. passenger strand) do not comprise flanking sequences.

According to one embodiment, a nucleic acid sequence of the miR-135 molecule (e.g. of miR-135b and/or of complementary strand) comprises one or more modification.

According to one embodiment, a nucleic acid sequence of the miR-135 molecule (e.g. of miR-135b and/or of complementary strand) comprises a single modification as compared to an endogenous mature miR-135b sequence or its complementary strand (also referred to as native miR-135b sequences).

According to another embodiment, the nucleic acid sequence of the miR-135 molecule (e.g. of miR-135b and/or of complementary strand) comprises 2, 3, 4, 5, 6, 7, 8, 9, 10, 11, 12, 13, 14, 15, 16 or more modifications as compared to native miR-135b sequences.

According to one embodiment, the nucleic acid sequence of the miR-135 molecule comprises 1-2 modifications as compared to native miR-135b sequences.

According to one embodiment, the nucleic acid sequence of the miR-135 molecule comprises 3-4 modifications as compared to native miR-135b sequences.

According to one embodiment, the nucleic acid sequence of the miR-135 molecule comprises 5-10 modifications as compared to native miR-135b sequences.

According to one embodiment, the nucleic acid sequence of the miR-135 molecule comprises 10-15 modifications as compared to native miR-135b sequences.

According to one embodiment, the nucleic acid sequence of the miR-135 molecule comprises 16-26 modifications as compared to native miR-135b sequences.

Thus, the synthetic miR-135 molecule of the invention can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting.

According to one embodiment, the synthetic miR-135 molecule comprises a modification selected from an insertion, deletion, substitution or point mutation of a nucleic acid, as long as the molecule retains at least about 90%, 95%, 99% or 100% of its the biological activity (e.g. miR-135 silencing activity).

According to one embodiment, the synthetic miR-135 molecule includes at least one base (e.g. nucleobase) modification or substitution. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications. Additional base modifications are described in Deleavey and Damha, Chemistry and Biology (2012) 19: 937-954, incorporated herein by reference.

According to one embodiment, the modification is a chemical modification.

According to one embodiment, the synthetic miR-135 molecule of the invention can have a chemical modification on a nucleotide in an internal (i.e., non-terminal) region having non-complementarity with the target nucleic acid. For example, a modified nucleotide can be incorporated into the region of a miRNA that forms a bulge. The modification can include a ligand attached to the miRNA, e.g., by a linker. The modification can, for example, improve pharmacokinetics or stability of the polynucleotide, or improve hybridization properties (e.g., hybridization thermodynamics) of the polynucleotide to a target nucleic acid.

In some embodiments, the orientation of a modification or ligand incorporated into or tethered to the bulge region of a polynucleotide is oriented to occupy the space in the bulge region. For example, the modification can include a modified base or sugar on the nucleic acid strand or a ligand that functions as an intercalator. These are preferably located in the bulge. The intercalator can be an aromatic, e.g., a polycyclic aromatic or heterocyclic aromatic compound. A polycyclic intercalator can have stacking capabilities, and can include systems with 2, 3, or 4 fused rings. In some embodiments, the orientation of a modification or ligand incorporated into or tethered to the bulge region of the polynucleotide is oriented to occupy the space in the bulge region. This orientation facilitates the improved hybridization properties or an otherwise desired characteristic of the polynucleotide.

In one embodiment, the synthetic miR-135 molecule can include an aminoglycoside ligand, which can cause the polynucleotide to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. In some embodiments the guanidine analog (the guanidinoglycoside) of an aminoglycoside ligand is tethered to a polynucleotide agent. In a guanidinoglycoside, the amine group on the amino acid is exchanged for a guanidine group. Attachment of a guanidine analog can enhance cell permeability of the polynucleotide.

For increased nuclease resistance and/or binding affinity to the target, the synthetic miR-135 molecule of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), e.g. inclusion of nucleic acid analogues in which the ribose ring is “locked” by a methylene bridge connecting the 2′-O atom and the 4′-C atom, ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to the target. The inclusion of pyranose sugars in the oligonucleotide backbone can also decrease endonucleolytic cleavage.

A miR-135 molecule can be further modified by including a 3′ cationic group, or by inverting the nucleoside at the terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

According to one embodiment, the 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the synthetic miR-135 molecule includes a modification that improves targeting. Examples of modifications that target oligonucleotide agents to particular cell types include carbohydrate sugars such as galactose, N-acetylgalactosamine, mannose; vitamins such as folates; other ligands such as RGDs and RGD mimics; and small molecules including naproxen, ibuprofen or other known protein-binding molecules (further discussed herein below).

According to one embodiment, the modification is selected from the group consisting of a sugar modification, a nucleobase modification, and an internucleotide linkage modification, as is broadly described herein under.

Specific examples of synthetic miR-135 molecules useful according to this aspect of the present invention include those containing modified backbones (e.g. sugar-phosphate backbones) or non-natural internucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050 and 8,017,763; as well as in U.S. Pat. Applic. No. 20100222413, all incorporated herein by reference.

According to one embodiment, the nucleic acid sequence of the miR-135 molecule (e.g. of miR-135b and/or of complementary strand) comprises a phosphorus-modified internucleotide linkage at the 5′ or 3′ end of the nucleotide sequence.

Exemplary internucleotide linkage modifications include, but are not limited to, phosphorothioate, chiral phosphorothioate, phosphorodithioate, phosphotriester, aminoalkyl phosphotriester, methyl phosphonate, alkyl phosphonate (including 3′-alkylene phosphonates), chiral phosphonate, phosphinate, phosphoramidate (including 3′-amino phosphoramidate), aminoalkylphosphoramidate, thionophosphoramidate, thionoalkylphosphonate, thionoalkylphosphotriester, boranophosphate (such as that having normal 3′-5′ linkages, 2′-5′ linked analogues of these, and those having inverted polarity wherein the adjacent pairs of nucleoside units are linked 3′-5′ to 5′-3′ or 2′-5′ to 5′-2′), boron phosphonate, phosphodiester, phosphonoacetate (PACE), morpholino, peptide nucleic acid (PNA) and threose nucleic acid (TNA). Various salts, mixed salts, and free acid forms of the above modifications can also be used. Additional internucleotide linkage modifications are described in Deleavey and Damha, Chemistry and Biology (2012) 19: 937-954, incorporated herein by reference.

According to a specific embodiment, the internucleotide linkage modification comprises a phosphorothioate.

According to one embodiment, the synthetic miR-135 molecule comprises at least one phosphorothioate linkage modification in the nucleic acid sequence of miR-135b or the complementary strand.

According to one embodiment, the synthetic miR-135 molecule comprises at least 2, 3, 4, 5, 6, 7, 8, 9, 10, 11, 12, 13, 14, 15, 16, 17, 18, 19, 20 or more phosphorothioate linkage modifications in the nucleic acid sequence of miR-135b or the complementary strand.

According to one embodiment, the synthetic miR-135 molecule comprises a phosphorothioate at the internucleotide linkage at the 5′ or 3′ end of the nucleotide sequence (e.g. in the last nucleotide at the 5′ end of the miR-135b nucleic acid sequence).

According to one embodiment, the synthetic miR-135 molecule comprises a phosphorothioate between the last two nucleotides at the 3′ end of the nucleic acid sequence of miR-135b or the complementary strand.

According to one embodiment, the synthetic miR-135 molecule comprises a phosphorothioate between the last two nucleotides at the 5′ end of the nucleic acid sequence of miR-135b or the complementary strand.

According to one embodiment, the synthetic miR-135 molecule comprises a phosphorothioate between the last two nucleotides at the 3′ end and between the last two nucleotides at the 5′ end of the nucleic acid sequence of miR-135b or the complementary strand.

According to one embodiment, the synthetic miR-135 molecule comprises a phosphorothioate in both the nucleic acid sequence of miR-135b and in the nucleic acid sequence of the complementary strand.

According to one embodiment, the synthetic miR-135 molecule comprises a boranophosphate at the internucleotide linkage at the 5′ or 3′ end of the nucleotide sequence (e.g. in the last nucleotide at the 5′ end of the miR-135b nucleic acid sequence).

According to one embodiment, the synthetic miR-135 molecule comprises a methyl phosphonate at the internucleotide linkage at the 5′ or 3′ end of the nucleotide sequence (e.g. in the last nucleotide at the 5′ end of the miR-135 nucleic acid sequence).

According to one embodiment, the synthetic miR-135 molecule comprises a phosphodiester at the internucleotide linkage at the 5′ or 3′ end of the nucleotide sequence (e.g. in the last nucleotide at the 5′ end of the miR-135b nucleic acid sequence).

According to a specific embodiment, the synthetic miR-135 molecule comprises a phosphate at the internucleotide linkage at the 5′ or 3′ end of the nucleotide sequence (e.g. in the last nucleotide at the 5′ end of the miR-135b nucleic acid sequence).

Alternatively, synthetic miR-135 molecule backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

According to one embodiment, the synthetic miR-135 molecule comprises at least one sugar modification (e.g. ribose modification).

According to one embodiment, the synthetic miR-135 molecule comprises at least 2, 3, 4, 5, 6, 7, 8, 9, 10, 11, 12, 13, 14, 15, 16, 17, 18, 19, 20 or more sugar modifications (e.g. ribose modification).

According to one embodiment, at least one nucleic acid of the miR-135 molecule (e.g. of miR-135b and/or of complementary strand) comprises a modification corresponding to position 2 of the ribose.

According to one embodiment, the sugar modification is in the last nucleotide at the 3′ end of the nucleic acid sequence of the miR-135b strand or the complementary strand.

According to one embodiment, the sugar modification is in the last nucleotide at the 5′ end of the nucleic acid sequence of the miR-135b strand or the complementary strand.

According to one embodiment, the sugar modification is in the last nucleotide at the 3′ end and at the 5′ end of the nucleic acid sequence of the miR-135b strand or the complementary strand.

According to a specific embodiment, the sugar modification is in the first 1-2 nucleotides at the 5′ end of the nucleic acid sequence of the miR-135b strand and/or in the last 1-3 nucleotides at the 3′ end of the nucleic acid sequence of the miR-135b strand.

According to a specific embodiment, the sugar modification is in the first two nucleotides at the 5′ end of the nucleic acid sequence of the complementary strand and/or in the last nucleotide at the 3′ end of the nucleic acid sequence of the complementary strand.

According to one embodiment, the sugar modification is in both the nucleic acid sequence of miR-135b and in the nucleic acid sequence of the complementary strand.

Exemplary sugar modifications include, but are not limited to, 2′-modified nucleotide, e.g., a 2′-deoxy, 2′-fluoro (2′-F), 2′-deoxy-2′-fluoro, 2′-O-methyl (2′-O-Me), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-Fluoroarabinooligonucleotides (2′-F-ANA), 2′-O—N-methylacetamido (2′-O-NMA), 2′-NH2 or a locked nucleic acid (LNA). Additional sugar modifications are described in Deleavey and Damha, Chemistry and Biology (2012) 19: 937-954, incorporated herein by reference.

According to one embodiment, the synthetic miR-135 molecule comprises at least one 2′-O-methyl (2′-O-Me)-modified nucleotide.

According to one embodiment, the synthetic miR-135 molecule comprises 2, 3, 4, 5, 6, 7, 8, 9, 10, 11, 12 or more 2′-O-methyl (2′-O-Me)-modified nucleotides.

According to one embodiment, all of the nucleotides of the synthetic miR-135 molecule include a 2′-O-methyl (2′-O-Me) modification.

According to a specific embodiment, the 2′-O-methyl (2′-O-Me)-modified nucleotide is at the 5′ end of the nucleic acid sequence of the miR-135b strand or the complementary strand.

According to a specific embodiment, the 2′-O-methyl (2′-O-Me)-modified nucleotide is at the 3′ end of the nucleic acid sequence of the miR-135b strand or the complementary strand.

According to a specific embodiment, the 2′-O-methyl (2′-O-Me)-modified nucleotide is at the last 2 nucleotides at the 5′ end of the nucleic acid sequence of the miR-135b strand or the complementary strand and/or at the last nucleotide at the 3′ end of the nucleic acid sequence of the miR-135b strand or the complementary strand.

According to one embodiment, the synthetic miR-135 molecule comprises at least one 2′-O-methoxyethyl (2′-O-MOE)-modified nucleotide.

According to one embodiment, the synthetic miR-135 molecule comprises 2, 3, 4, 5, 6, 7, 8, 9, 10, 11, 12 or more 2′-O-methoxyethyl (2′-O-MOE)-modified nucleotides.

According to a specific embodiment, the 2′-O-methoxyethyl (2′-O-MOE)-modified nucleotide is at the 5′ end of the nucleic acid sequence of the miR-135b strand or the complementary strand.

According to a specific embodiment, the 2′-O-methoxyethyl (2′-O-MOE)-modified nucleotide is at the 3′ end of the nucleic acid sequence of the miR-135b strand or the complementary strand.

According to a specific embodiment, the 2′-O-methoxyethyl (2′-O-MOE)-modified nucleotide is at the last 2 nucleotides at the 3′ end of the nucleic acid sequence of the miR-135b strand or the complementary strand and/or at the last nucleotide at the 5′ end of the nucleic acid sequence of the miR-135b strand or the complementary strand.

According to a specific embodiment, the 2′-O-MOE modification is effected on an adenine (A) nucleotide of the miR-135b strand or the complementary strand (e.g. at the 3′ end of the nucleic acid sequence of the miR-135b strand).

According to a specific embodiment, the 2′-O-MOE modification further comprises a 5′ ribose methylation (designated 2′-O-MOE-5′-Me).

According to a specific embodiment, the 2′-O-MOE-5′-Me modification is effected on a uracil (U) nucleotide of the miR-135b strand or the complementary strand (e.g. at the 5′ end of the nucleic acid sequence of the miR-135b strand).

According to one embodiment, the synthetic miR-135 molecule comprises at least one 2′-fluoro (2′-F)-modified nucleotide.

According to one embodiment, the synthetic miR-135 molecule comprises 2, 3, 4, 5, 6, 7, 8, 9, 10, 11, 12 or more 2′-fluoro (2′-F)-modified nucleotides.

According to a specific embodiment, the 2′-fluoro (2′-F)-modified nucleotide is at the 5′ end of the nucleic acid sequence of the miR-135b strand or the complementary strand.

According to a specific embodiment, the 2′-fluoro (2′-F)-modified nucleotide is not the end nucleotide at the 5′ end or the 3′ end of the nucleic acid sequence of the miR-135b strand or the complementary strand.

According to one embodiment, the synthetic miR-135 molecule comprises a modified internucleotide linkage and a sugar modification. Accordingly, the synthetic miR-135 molecules which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

According to one embodiment, the synthetic miR-135 molecule comprises a phosphorus-modified internucleotide linkage and at least one sugar modification (e.g. 2′-modified nucleotide).

According to one embodiment, the synthetic miR-135 molecule comprises a phosphorus-modified internucleotide linkage, at least one phosphorothioate-modified internucleotide linkage, and at least one sugar modification (e.g. 2′-modified nucleotide).

According to a specific embodiment, the synthetic miR-135 molecule comprises a phosphate in the last nucleotide at the 5′ end of the miR-135b nucleic acid sequence and a 2′-O-Me modification in the last nucleotide at the 3′ end of the miR-135b nucleic acid sequence.

According to a specific embodiment, the synthetic miR-135 molecule comprises a phosphate in the last nucleotide at the 5′ end of the miR-135b nucleic acid sequence, a 2′-O-Me modification in the last two nucleotides at the 5′ end of the miR-135b nucleic acid sequence and a 2′-O-Me modification in the last nucleotide at the 3′ end of the miR-135b nucleic acid sequence.

According to a specific embodiment, the synthetic miR-135 molecule comprises a phosphate in the last nucleotide at the 5′ end of the miR-135b nucleic acid sequence, a 2′-O-MOE-5′-Me modification in the last nucleotide at the 5′ end of the miR-135b nucleic acid sequence, and a 2′-O-Me modification in the last nucleotide at the 3′ end of the miR-135b nucleic acid sequence.

According to a specific embodiment, the synthetic miR-135 molecule comprises a phosphate in the last nucleotide at the 5′ end of the miR-135b nucleic acid sequence, a 2′-O-MOE-5′-Me modification in the last nucleotide at the 5′ end of the miR-135b nucleic acid sequence, and a 2′-O-MOE modification in the last two nucleotides at the 3′ end of the miR-135b nucleic acid sequence.

According to a specific embodiment, the synthetic miR-135 molecule comprises a 2′-O-Me modification in the last nucleotide at the 3′ end of the complementary strand nucleic acid sequence and a 2′-O-Me modification in the last two nucleotides at the 5′ end of the complementary strand nucleic acid sequence.

According to one embodiment, any of the above-described synthetic miR-135 molecules further comprise at least one 2′-F-modified nucleotide.

According to one embodiment, any of the above-described synthetic miR-135 molecules further comprise at least one phosphorothioate-modified internucleotide linkage.

Exemplary synthetic miR-135b sequences include, but are not limited to, SEQ ID NOs: 1, 5, 7, 8, 10, 14, 16, 41, 42, 43, 44, 45 and 46.

Exemplary modified complementary sequences include, but are not limited to, SEQ ID NOs: 2, 3, 4, 6, 9, 11, 12, 13, 15 and 47.

According to a specific embodiment, the synthetic miR-135 molecule of the present invention has a nucleic acid sequence of miR-135b as set forth in SEQ ID NO: 10.

According to a specific embodiment, the synthetic miR-135 molecule of the present invention has a nucleic acid sequence of miR-135b as set forth in SEQ ID NO: 16.

According to a specific embodiment, the synthetic miR-135 molecule of the present invention has a nucleic acid sequence of miR-135b as set forth in SEQ ID NO: 41.

According to a specific embodiment, the synthetic miR-135 molecule of the present invention has a nucleic acid sequence of miR-135b as set forth in SEQ ID NO: 42.

According to a specific embodiment, the synthetic miR-135 molecule of the present invention has a nucleic acid sequence of the complementary strand as set forth in SEQ ID NO: 13.

According to a specific embodiment, the synthetic miR-135 molecule of the present invention has a nucleic acid sequence of the complementary strand as set forth in SEQ ID NO: 47.

According to a specific embodiment, the synthetic miR-135 molecule of the present invention comprises a nucleic acid sequence of a miR-135b as set forth in SEQ ID NO: 37, and a complementary strand as set forth in SEQ ID NO: 40.

According to a specific embodiment, the synthetic miR-135 molecule of the present invention comprises a nucleic acid sequence of a miR-135b as set forth in SEQ ID NO: 10, and a complementary strand as set forth in SEQ ID NO: 13.

According to a specific embodiment, the synthetic miR-135 molecule of the present invention comprises a nucleic acid sequence of a miR-135b as set forth in SEQ ID NO: 41, and a complementary strand as set forth in SEQ ID NO: 13.

According to a specific embodiment, the synthetic miR-135 molecule of the present invention comprises a nucleic acid sequence of a miR-135b as set forth in SEQ ID NO: 42, and a complementary strand as set forth in SEQ ID NO: 13.

According to a specific embodiment, the synthetic miR-135 molecule of the present invention comprises a nucleic acid sequence of a miR-135b as set forth in SEQ ID NO: 43, and a complementary strand as set forth in SEQ ID NO: 13.

According to a specific embodiment, the synthetic miR-135 molecule of the present invention comprises a nucleic acid sequence of a miR-135b as set forth in SEQ ID NO: 44, and a complementary strand as set forth in SEQ ID NO: 13.

According to a specific embodiment, the synthetic miR-135 molecule of the present invention comprises a nucleic acid sequence of a miR-135b as set forth in SEQ ID NO: 45, and a complementary strand as set forth in SEQ ID NO: 13.

According to a specific embodiment, the synthetic miR-135 molecule of the present invention comprises a nucleic acid sequence of a miR-135b as set forth in SEQ ID NO: 46, and a complementary strand as set forth in SEQ ID NO: 13.

According to a specific embodiment, the synthetic miR-135 molecule of the present invention comprises a nucleic acid sequence of a miR-135b as set forth in SEQ ID NO: 16, and a complementary strand as set forth in SEQ ID NO: 13.

According to a specific embodiment, the synthetic miR-135 molecule of the present invention comprises a nucleic acid sequence of a miR-135b as set forth in SEQ ID NO: 10, and a complementary strand as set forth in SEQ ID NO: 47.

According to a specific embodiment, the synthetic miR-135 molecule of the present invention comprises a nucleic acid sequence of a miR-135b as set forth in SEQ ID NO: 41, and a complementary strand as set forth in SEQ ID NO: 47.

According to a specific embodiment, the synthetic miR-135 molecule of the present invention comprises a nucleic acid sequence of a miR-135b as set forth in SEQ ID NO: 42, and a complementary strand as set forth in SEQ ID NO: 47.

According to a specific embodiment, the synthetic miR-135 molecule of the present invention comprises a nucleic acid sequence of a miR-135b as set forth in SEQ ID NO: 43, and a complementary strand as set forth in SEQ ID NO: 47.

According to a specific embodiment, the synthetic miR-135 molecule of the present invention comprises a nucleic acid sequence of a miR-135b as set forth in SEQ ID NO: 44, and a complementary strand as set forth in SEQ ID NO: 47.

According to a specific embodiment, the synthetic miR-135 molecule of the present invention comprises a nucleic acid sequence of a miR-135b as set forth in SEQ ID NO: 45, and a complementary strand as set forth in SEQ ID NO: 47.

According to a specific embodiment, the synthetic miR-135 molecule of the present invention comprises a nucleic acid sequence of a miR-135b as set forth in SEQ ID NO: 46, and a complementary strand as set forth in SEQ ID NO: 47.

According to a specific embodiment, the synthetic miR-135 molecule of the present invention comprises a nucleic acid sequence of a miR-135b as set forth in SEQ ID NO: 16, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 10, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 41, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 42, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 43, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 44, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 45, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 46, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 16, and a complementary strand as set forth in SEQ ID NO: 13.

According to one aspect of the invention, there is provided a composition of matter comprising a synthetic miR-135 molecule comprising a nucleic acid sequence of a miR-135b as set forth in SEQ ID NO: 10, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 41, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 42, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 43, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 44, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 45, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 46, and a complementary strand as set forth in SEQ ID NO: 47.

According to one aspect of the invention, there is provided a composition of matter comprising a synthetic miR-135 molecule comprising a nucleic acid sequence of a miR-135b as set forth in SEQ ID NO: 16, and a complementary strand as set forth in SEQ ID NO: 47.

The synthetic miR-135 molecules of the invention can be constructed using chemical synthesis and/or enzymatic ligation reactions using procedures known in the art (as discussed in detail below). For example, the polynucleotide can be chemically synthesized using naturally occurring nucleotides or variously modified nucleotides designed to increase the biological stability of the molecules or to increase the physical stability of the duplex formed between the polynucleotide and target nucleic acids, e.g., phosphorothioate derivatives and acridine substituted nucleotides can be used (as discussed in detail hereinabove).

The synthetic miR-135 molecule designed according to the teachings of the present invention can be generated according to any oligonucleotide synthesis method known in the art, including both enzymatic syntheses and solid-phase syntheses. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

According to one embodiment, chemical synthesis can be achieved by the diester method, triester method, polynucleotides phosphorylase method and by solid-phase chemistry. These methods are discussed in further detail below.

Diester Method: The diester method was the first to be developed to a usable state. The basic step is the joining of two suitably protected deoxynucleotides to form a dideoxynucleotide containing a phosphodiester bond.

Triester Method: The main difference between the diester and triester methods is the presence in the latter of an extra protecting group on the phosphate atoms of the reactants and products. The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide Phosphorylase Method: This is an enzymatic method of DNA synthesis that can be used to synthesize many useful oligonucleotides. Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-Phase Methods: Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers. Equipment and reagents for executing solid-phase synthesis are commercially available from, for example, Applied Biosystems.

Phosphoramidite chemistry has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Recombinant Methods: Recombinant methods for producing nucleic acids in a cell are well known to those of skill in the art and can be implemented in cases where the synthetic miR-135 molecule does not comprise chemical modifications. These include the use of vectors, plasmids, cosmids, and other vehicles for delivery a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Maryland; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to one embodiment, the synthetic miR-135 molecule is attached to a ligand (also referred to as moiety) that is selected to improve stability, distribution, cellular uptake, crossing of the blood brain barrier (BBB) or to target the synthetic miR-135 molecule to a cell of interest. Thus, the synthetic miR-135 molecule may be modified to include a non-nucleotide moiety, as discussed in detail below.

According to one aspect of the present invention there is provided a conjugated miR-135 molecule.

The term “conjugate” as used herein refers to any compound resulting from the covalent attachment of two or more individual compounds. In the present invention, a conjugate refers to a molecule comprising a synthetic miR-135 molecule and a cell-targeting moiety which are covalently coupled.

As used herein, the expression “cell-targeting moiety” refers to any substance that binds to a molecule expressed or presented on the target cell of interest, preferably in a specific manner, e.g. not expressed/presented on other cell types or expressed/presented at higher levels than in other cell types. According to a specific embodiment, the molecule is a receptor. This binding specificity allows the delivery of the synthetic miR-135 molecule (which is attached to the cell-targeting moiety) to the cell, tissue or organ that expresses or presents the molecule. In this way, a conjugate carrying cell-targeting moiety will be directed specifically to the cells when administered to a subject (e.g. human) or contacted in vitro with a population of cells of different types.

A cell-targeting moiety according to the present invention may show a Kd for the target (the molecule expressed or presented on the target cell of interest, e.g. receptor) of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M or greater.

The term “receptor” refers to a cell-associated protein that binds to a bioactive molecule termed a “ligand”.

According to one embodiment, the molecule expressed or presented on the target cell of interest (e.g. receptor) is expressed in a cell-specific manner (e.g. on central nervous system cells, bone cells, muscle cells, cancer cells, gastrointestinal cells, etc.).

Receptors which may be targeted by the cell-targeting moiety of the invention include, without being limited to, a 5-hydroxytryptamine receptor (e.g. 5-HT1A, 5-HT1B, 5-HT2A, 5-HT3, 5-HT1D, 5-HT6), an adenosine receptor (e.g. A1, A2 A2A), an adrenoceptor receptor (e.g. alpha 1A-adrenoceptor, alpha 1B-adrenoceptor, alpha 1D-adrenoceptor), an angiotensin receptor (e.g. AT2), a bombesin receptor (e.g. BB1, BB2, BB3), a bradykinin receptor (e.g. B1, B2), a calcitonin receptor (e.g. AM1, AMY1, CGRP, CT-R, AM2, AMY3), a chemokine receptor (e.g. CXCR4), a cholecystokinin receptor (e.g. CCK2), a corticotropin-releasing factor receptor (e.g. CRF1, CRF2), a dopamine receptor (e.g. D1, D2), an endothelin receptor (e.g. Eta, Etp), an ephrin receptor (e.g. EphA1, EphA2, EphA3, EphA4, EphB1, EphB2, EphB3), a formylpeptide receptor (e.g. FPR1, FPR2, FPR3), a Frizzled receptor (e.g. FZD2, FZD3, FZD4, FZD5, FZD6, FZD7, FZD8, FZD9, FZD10), a galanin receptor (e.g. GAL1, GAL2, GAL3), a growth hormone secretagogue receptor (Ghrelin) (e.g. GHS-R1a), a Kisspeptin receptor, a melanocortin receptor (e.g. MC1, MC2, MC3, MC4), a melatonin receptor (e.g. MT1, MT2), Neuropeptide FF/neuropeptide AF receptor (e.g NPFF1, NPFF2), a neuropeptide S receptor (e.g. NPS), a neuropeptide W/neuropeptide B receptor (e.g. NPBW2), a neuropeptide Y receptor (e.g. Y1, Y2, Y4, Y5), a neurotensin receptor (e.g. NTS1, NTS2), an opioid receptor (e.g. delta, kappa, mu), an orexin receptor (e.g. OX1, OX2), a peptide P518 receptor (e.g. QRFP), a prostanoid receptor, a SLC6 neurotransmitter transporter family (e.g. DAT, NET, SERT, GlyT1), a somatostatin receptor (e.g. sst1, sst2, sst3, sst4, sst5), a tachykinin receptor (e.g. NK1, NK2, NK3), a Toll-like receptor (e.g. TLR7), a vasopressin and oxytocin receptor (e.g. OT, V1A, V1B, V2), a VEGF receptor (e.g. VEGFR1, VEGFR2, VEGFR3) and a G-protein coupled receptor (GPCR).

According to one embodiment, the molecule expressed or presented on the target cell of interest (e.g. receptor) is expressed/presented on cells of the central nervous system (CNS) including, but not limited to, cells of the hypothalamus, brainstem, cortex, cerebellum, striatum, mesencephalon, hippocampus, glia and/or spinal cord.

According to one embodiment, the molecule (e.g. receptor) is expressed or presented on brain cells.

According to one embodiment, the molecule (e.g. receptor) is expressed or presented on neuronal cells.

According to one embodiment, the molecule (e.g. receptor) is expressed or presented on glial cells (neuroglia).

According to one embodiment, the molecule expressed or presented on the target cell of interest (e.g. receptor) is a neurotransmitter transporter.

According to one embodiment, the second component of the conjugate according to the present invention is a cell-targeting moiety that binds specifically to a neurotransmitter transporter.

As used herein, the term “neurotransmitter transporter”, as used herein, refers to a protein belonging to a class of membrane transport proteins that span the cellular membranes of neurons and which primary function is to carry neurotransmitters across these membranes and to direct their further transport to specific intracellular locations.

Neurotransmitter transporters which may be targeted by the cell-targeting moiety of some embodiments of the invention include, without being limited to, uptake carriers present in the plasma membrane of neurons and glial cells, which pump neurotransmitters from the extracellular space into the cell. This process relies on the Na+ gradient across the plasma membrane, particularly the co-transport of Na⁺. Two families of proteins have been identified. One family includes the transporters for GABA, monoamines such as noradrenaline, dopamine, serotonin, and amino acids such as glycine and proline. Common structural components include twelve putative transmembrane a-helical domains, cytoplasmic N- and C-termini, and a large glycosylated extracellular loop separating transmembrane domains three and four. This family of homologous proteins derives their energy from the co-transport of Na⁺ and Cl⁻ ions with the neurotransmitter into the cell (Na/Cf neurotransmitter transporters). The second family includes transporters for excitatory amino acids such as glutamate. Common structural components include putative 6-10 transmembrane domains, cytoplasmic N- and C-termini, and glycosylations in the extracellular loops. The excitatory amino acid transporters are not dependent on Cl—, and may require intracellular K+ ions (Na+/K+-neurotransmitter transporters) (Liu, Y. et al. (1999) Trends Cell Biol. 9: 356-363).

Neurotransmitter transporters which may be targeted by the cell-targeting moiety of the invention include, without being limited to, uptake carriers present in the plasma membrane of neurons and glial cells, which pump neurotransmitters from the extracellular space into the cell. This process relies on the Na⁺ gradient across the plasma membrane, particularly the co-transport of Na⁺. Two families of proteins have been identified. One family includes the transporters for GABA, monoamines such as noradrenaline, dopamine, serotonin, and amino acids such as glycine and proline. Common structural components include twelve putative transmembrane a-helical domains, cytoplasmic N- and C-termini, and a large glycosylated extracellular loop separating transmembrane domains three and four. This family of homologous proteins derives their energy from the co-transport of Na⁺ and Cl⁻ ions with the neurotransmitter into the cell (Na/Cl neurotransmitter transporters). The second family includes transporters for excitatory amino acids such as glutamate. Common structural components include putative 6-10 transmembrane domains, cytoplasmic N- and C-termini, and glycosylations in the extracellular loops. The excitatory amino acid transporters are not dependent on Cl⁻, and may require intracellular K⁺ ions (Na⁺/K⁺-neurotransmitter transporters) (Liu, Y. et al. (1999) Trends Cell Biol. 9: 356-363).

Neurotransmitter transporters which may be targeted by the cell-targeting moiety of the invention also include neurotransmitter transporters present in intracellular vesicle membranes, typically synaptic vesicles, which primary function is concentrating neurotransmitters from the cytoplasm into the vesicle, before exocytosis of the vesicular contents during synaptic transmission. Vesicular transport uses the electrochemical gradient across the vesicular membrane generated by an H⁺-ATPase. Two families of proteins are involved in the transport of neurotransmitters into vesicles. One family uses primarily proton exchange to drive transport into secretory vesicles and includes the transporters for monoamines and acetylcholine. For example, the monoamine transporters exchange two luminal protons for each molecule of cytoplasmic transmitter. The second family includes the GABA transporters, which relies on the positive charge inside synaptic vesicles. The two classes of vesicular transporters show no sequence similarity to each other and have structures distinct from those of the plasma membrane carriers (Schloss, P. et al. (1994) Curr. Opin. Cell Biol. 6: 595-599; Liu, Y. et al. (1999) Trends Cell Biol. 9: 356-363).

According to one embodiment, types of neurotransmitter transporters that can be targeted with the cell-targeting moiety of the invention include e.g. dopamine transporters (DAT), serotonin transporters (SERT) and norepinephrine transporters (NET).

Dopamine transporter (also termed DAT or SLC6A3) refers to a molecule which is an integral membrane protein that transports the neurotransmitter dopamine from the synaptic cleft and deposits it into surrounding cells, thus terminating the signal of the neurotransmitter.

Serotonin transporter (also termed SERT or SLC6A4) refers to a polypeptide which is an integral membrane protein that transports the neurotransmitter serotonin from synaptic spaces into presynaptic neurons.

Norepinephrine transporter (also termed NET or SLC6A2) refers to a molecule which is a transmembrane protein that transports synaptically released norepinephrine back into the presynaptic neuron.

Specific types of neurotransmitter transporters that can be targeted with the cell-targeting moiety of the invention include, but are not limited to, glutamate/aspartate transporters, including, excitatory amino acid transporter 1 (EAAT1), excitatory amino acid transporter 2 (EAAT2), excitatory amino acid transporter 3 (EAAT3), excitatory amino acid transporter 4 (EAAT4), excitatory amino acid transporter 5 (EAAT5), vesicular glutamate transporter 1 (VGLUT1), vesicular glutamate transporter 2 (VGLUT2) and vesicular glutamate transporter 3 (VGLUT3); GABA transporters, including, GABA transporter type 1 (GAT1), GABA transporter type 2 (GAT2), GABA transporter type 3 (GAT3), Betaine transporter (BGT1) and vesicular GABA transporter (VGAT); glycine transporters, including, glycine transporter type 1 (GlyT1), glycine transporter type 2 (GlyT2); monoamine transporters, including, dopamine transporter (DAT), norepinephrine transporter (NET), serotonin transporter (SERT), vesicular monoamine transporter 1 (VMAT1), vesicular monoamine transporter 2 (VMAT2); adenosine transporters, including, equilibrative nucleoside transporter 1 (ENT1), equilibrative nucleoside transporter 2 (ENT2), equilibrative nucleoside transporter 3 (ENT3) and equilibrative nucleoside transporter 4 (ENT4) and vesicular acetylcholine transporter (VAChT).

According to one embodiment, the conjugate of the invention comprises a cell-targeting moiety that binds specifically to a tumor associated antigen.

As used herein the phrase “tumor-associated antigen” refers to a protein that is common to a specific hyperproliferative disorder (such as cancer) and is produced by the tumor cells.

The type of tumor-associated antigen referred to in the invention includes a tumor-specific antigen (TSA) or a tumor-associated antigen (TAA). A “TSA” refers to a protein or polypeptide antigen unique to tumor cells and which does not occur on other cells in the body. A “TAA” refers to a protein or polypeptide antigen that is expressed by a tumor cell. For example, a TAA may be one or more surface proteins or polypeptides, nuclear proteins or glycoproteins, or fragments thereof, of a tumor cell.

According to one embodiment, the tumor-associated antigen is associated with a solid tumor (e.g. colon carcinoma, breast carcinoma, prostate carcinoma, renal cell carcinoma (RCC), lung carcinoma, sarcoma or melanoma).

According to one embodiment, the tumor-associated antigen is associated with a hematologic malignancy.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.291\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS. Further examples of tumor antigens include, but are not limited to, A33, BAGE, Bcl-2, β-catenin, CA125, CA19-9, CD5, CD19, CD20, CD21, CD22, CD33, CD37, CD45, CD123, CEA, c-Met, CS-1, cyclin B1, DAGE, EBNA, EGFR, ephrinB2, estrogen receptor, FAP, ferritin, folate-binding protein, GAGE, G250, GD-2, GM2, gp75, gp100 (Pmel 17), HER-2/neu, HPV E6, HPV E7, Ki-67, LRP, mesothelin, p53 and PRAME. Further tumor antigens are provided in van der Bruggen P, Stroobant V, Vigneron N, Van den Eynde B. Peptide database: T cell-defined tumor antigens. Cancer Immun (2013), www(dot)cancerimmunity(dot)org/peptide/, incorporated herein by reference.

According to one embodiment, the conjugate of the invention comprises a cell-targeting moiety that binds specifically to a molecule (e.g. receptor) expressed or presented on bone cells.

According to one embodiment, the cell-targeting moiety targets the skeletal system.

According to one embodiment, the cell-targeting moiety is aimed at a specific bone cell type (e.g. osteoblast, osteocyte, osteoclast, bone cell progenitor, osteoclast progenitor or a bone lining cell).

Exemplary bone cell targets which can be targeted by the cell-targeting moiety of the invention, include but are not limited to, hydroxyapatite (HA), osteocalcin, bone sialoprotein, collagen type 1, bone alkaline phosphatase, dentine matrix protein 1 and sclerostin.

According to one embodiment, the conjugate of the invention comprises a cell-targeting moiety that binds specifically to a molecule (e.g. receptor) expressed or presented on muscle cells.

Exemplary muscle cell molecules which can be targeted by the cell-targeting moiety of the invention, include but are not limited to, M-Cadherin/Cadherin-15, Caveolin-1, ABCG2, and Myogenin.

According to one embodiment, the conjugate of the invention comprises a cell-targeting moiety that binds specifically to a molecule (e.g. receptor) expressed or presented on gastrointestinal cells.

Exemplary gastrointestinal cell molecules which can be targeted by the cell-targeting moiety of the invention, include but are not limited to, serotonin transporter or serotoning receptor 1-7.

The selection of the cell-targeting moiety of the invention will depend on the particular type of disease (e.g. psychiatric disorder, cancer, bone disease, etc.) to be treated.

According to one embodiment, the cell-targeting moiety is a small molecule.

According to one embodiment, the cell-targeting moiety is a small drug. Exemplary small drugs which can be used to target e.g. 5-HT neurons and post-synaptic neurons, include but are not limited to, ligands for 5-HT1a receptor [11C] DASB, [11C] WAY100635 or [18F] MPPF.

According to one embodiment, the cell-targeting moiety is a synthetic component.

According to one embodiment, the cell-targeting moiety is a nanoparticle capable of binding an antigen, a receptor or other protein, or non-proteinaceous membrane compounds of a target cell.

According to one embodiment, the cell-targeting moiety is an affinity binding moiety, i.e. any naturally occurring or artificially produced molecule or composition which binds to a specific molecule (e.g. antigen) with a higher affinity than to a non-specific molecule (e.g. antigen).

It should be noted that the affinity can be quantified using known methods such as, Surface Plasmon Resonance (SPR) (described in Scarano S, Mascini M, Turner A P, Minunni M. Surface plasmon resonance imaging for affinity-based biosensors. Biosens Bioelectron. 2010, 25: 957-66) using e.g. a captured or immobilized monoclonal antibody (MAb) format to minimize contribution of avidity, and can be calculated using, e.g., a dissociation constant, Kd, such that a lower Kd reflects a higher affinity.

An affinity binding moiety typically has a binding affinity (K_{D}) of at least about 2 to about 200 M (i.e. as long as the binding is specific i.e., no background binding),

According to a specific embodiment, the affinity binding moiety is an aptamer or a lectin.

According to a specific embodiment, the affinity binding moiety is an antibody or an antibody fragment.

The term “antibody” as used in this invention includes intact molecules as well as functional fragments thereof, such as Fab, Fab′, F(ab′)2, Fv, linear antibodies, scFv antibodies, and multispecific antibodies formed from antibody fragments that are capable of binding to the antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

According to one embodiment, the cell-targeting moiety binds to a neurotransmitter transporter.

According to one embodiment, the cell-targeting moiety which binds specifically to a neurotransmitter transporter is selected from the group consisting of a serotonin reuptake inhibitors (SRI), a selective serotonin reuptake inhibitor (SSRI), a serotonin-norepinephrine reuptake inhibitor (SNRI), a noradrenergic and specific serotoninergic antidepressant (NASSA), a noradrenaline reuptake inhibitor (NRI), a dopamine reuptake inhibitor (DRI), an endocannabinoid reuptake inhibitor (eCBRI), an adenosine reuptake inhibitor (AdoRI), an excitatory Amino Acid Reuptake Inhibitor (EAARI), a glutamate reuptake inhibitor (GluRI), a GABA Reuptake Inhibitor (GRI), a glycine Reuptake Inhibitor (GlyRI), a Norepinephrine-Dopamine Reuptake Inhibitor (NDRI), a triple reuptake inhibitor, a noradrenaline dopamine double reuptake inhibitor, a serotonin single reuptake inhibitor, a noradrenaline single reuptake inhibitor and a dopamine single reuptake inhibitor.

The term “serotonin reuptake inhibitor” or “SRI” refers to a molecule which is capable of blocking serotonin uptake and includes both selective serotonin reuptake inhibitors (SSRI) (which block specifically serotonin uptake without substantially affecting other neurotransmitter) as well as non-selective serotonin reuptake inhibitors such as serotonin-norepinephrine reuptake inhibitors (SNRI) and serotonin-norepinephrine-dopamine reuptake inhibitors (SNDRI).

The term “serotonin selective reuptake inhibitors” or “SSRI” refers to selective inhibitors of serotonin reuptake without substantially affecting other neurotransmitter reuptake or transporter systems. These compounds act primarily at the presynaptic serotoninergic cell leading to an increase in the extracellular level of the neurotransmitter serotonin, thereby increasing the level of serotonin available to bind to the postsynaptic receptor and reversing the deficit of the activity of this monoaminergic neurotransmitter system in the brain. Exemplary non-limiting examples of SSRI include, but are not limited to, sertraline (CAS 79617-96-2), a sertraline-structural analog, fluoxetine (CAS 54910-89-3), fluvoxamine (CAS 54739-18-3), paroxetine (CAS 61869-08-7), indapline (CAS 63758-79-2), zimeldine (CAS 56775-88-3), citalopram (CAS 59729-33-8) and escitalopram (CAS 219861-08-2). Any method known in the art can determine whether a given compound acts as a SSRI, these include but are not limited to, assaying the ability to reduce ex vivo uptake of serotonin and of antagonizing the serotonin-depleting action of p-chloroamphetamine without affecting rat heart uptake of intravenous [³H]norepinephrine as described in Koe et al. (J. Pharmacol. Exp. Ther., 1983, 226:686-700).

In a specific embodiment, the SSRI is sertraline or a structural analog thereof having the structure (Formula I)

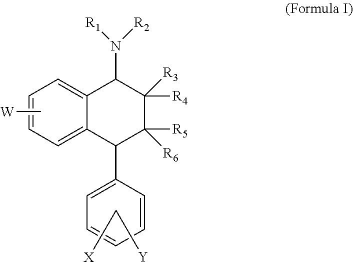

wherein, independently, R₁, R₂, R₃, R₄, R₅, and R₆ are hydrogen or an optionally substituted C1-C6 alkyl; X and Y are each selected from the group consisting of hydrogen, fluoro, chloro, bromo, trifluoromethyl, C1-C3 alkoxy, and cyano; and W is selected from the group consisting of hydrogen, fluoro, chloro, bromo, trifluoromethyl, nitro and C1-C3 alkoxy. In some embodiments, the sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and the 4-carbons are asymmetrically substituted, each cis-compound has two optically active enantiomeric forms denoted (with reference to the I-carbon) as the cis-(1R) and cis-(1S) enantiomers.

Certain useful sertraline analogs are the following compounds, in either the (1S)-enantiomeric or the (1S)(1R) racemic forms, and their pharmaceutically acceptable salts:
cis-N-methyl-4-(3,4-dichlorophenyl)-1,2,3,4-tetrahydro-1-naphthalenamine;
cis-N-methyl-4-(4-bromophenyl)-1,2,3,4-tetrahydro-1-naphthalenamine;
cis-N-methyl-4-(4-chlorophenyl)-1,2,3,4-tetrahydro-1-naphthalenamine;
cis-N-methyl-4-(3-trifluoromethyl-phenyl)-1,2,3,4-tetrahydro-1-naphthalenamine;
cis-N-methyl-4-(3-trifluoromethyl-4-chlorophenyl)-1,2,3,4-tetrahydro-1-naphthalenamine;
cis-N,N-dimethyl-4-(4-chlorophenyl)-1,2,3,4-tetrahydro-1-naphthalenamine;
cis-N,N-dimethyl-4-(3-trifluoromethyl-phenyl)-1,2,3,4-tetrahydro-1-naphthalenamine and
cis-Nmethyl-4-(4-chlorophenyl)-7-chloro-1,2,3,4-tetrahydro-1-naphthalenamine.

Of interest also is the (1R)-enantiomer of cis-N-methyl-4-(3,4-dichlorophenyl)-1,2,3,4-tetrahydro-1-naphthalenamine.

Sertraline analogs are also described in U.S. Pat. No. 4,536,518 (incorporated herein by reference). Other related compounds include (S,S)—N-desmethylsertraline, rac-cis-N-desmethylsertraline, (1S,4S)-desmethyl sertraline, 1-des(methylamine)-1-oxo-2-(R,S)-hydroxy sertraline, (1R,4R)-desmethyl sertraline, sertraline, sulfonamide, sertraline (reverse) methane sulfonamide, 1R,4R sertraline, enantiomer, N,N-dimethyl sertraline, nitro sertraline, sertraline aniline, sertraline iodide, sertraline sulfonamide NH2, sertraline sulfonamide ethanol, sertraline nitrile, sertraline-CME, dimethyl sertraline reverse sulfonamide, sertraline reverse sulfonamide (CH2linker), sertraline B-ring ortho methoxy, sertraline A-ring methyl ester, sertraline A-ring ethanol, sertraline N,Ndimethylsulfonamide, sertraline A ring carboxylic acid, sertraline B-ring paraphenoxy, sertraline B-ring para-trifluoromethane, N,N-dimethyl sertraline B-Ring and para-trifluoromethane, and UK-416244. Structures of these analogs are shown below.

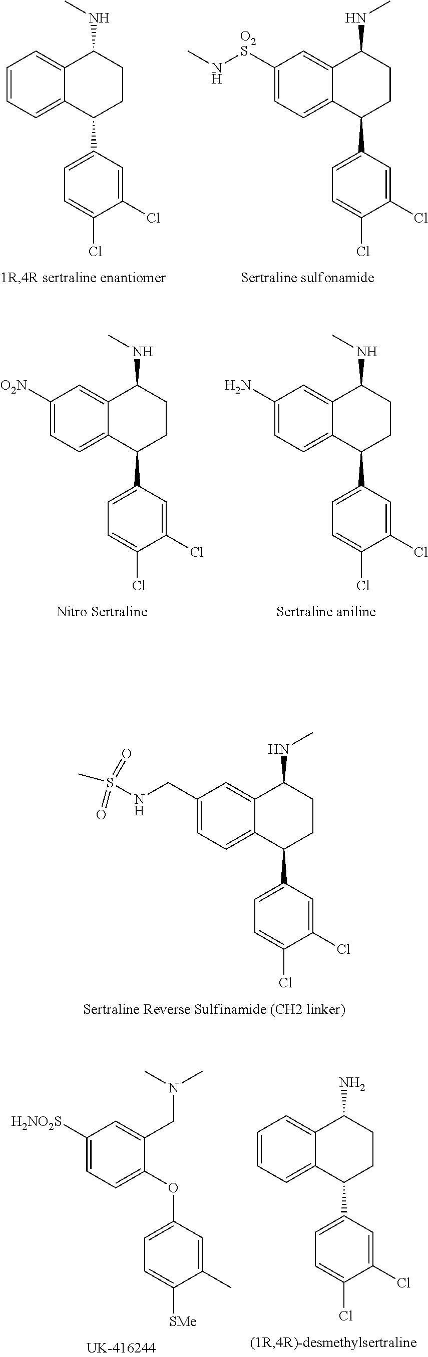

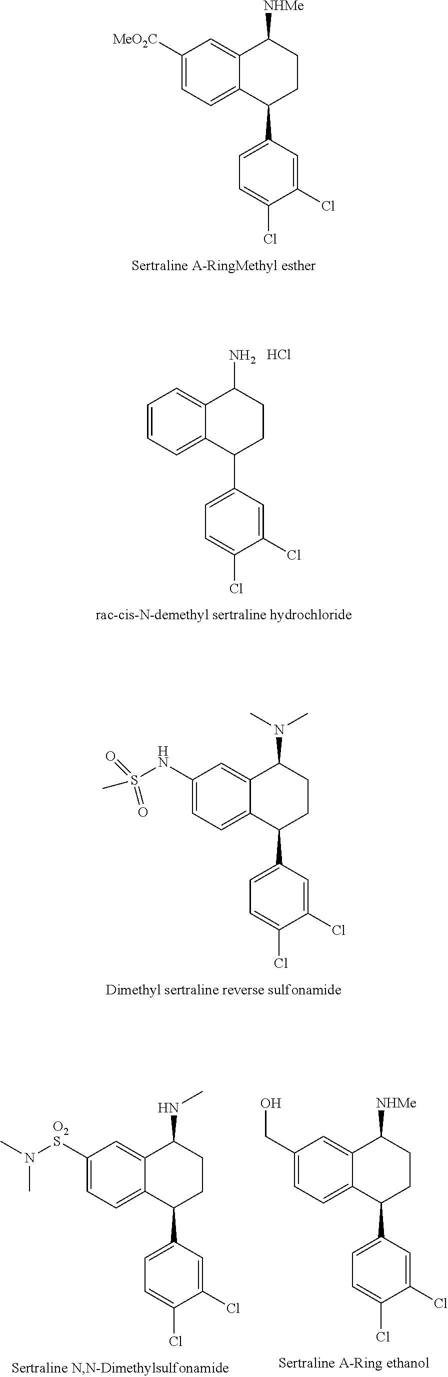 [SMILES: CN[C@H]1CC[C@@H](c2ccc(Cl)c(Cl)c2)c2ccc(C(=O)OC)cc21 ; Cl ; NC1CCC(c2ccc(Cl)c(Cl)c2)c2ccccc21 ; CN(C)[C@H]1CC[C@@H](c2ccc(Cl)c(Cl)c2)c2ccc(NS(C)(=O)=O)cc21 ; CN[C@H]1CC[C@@H](c2ccc(Cl)c(Cl)c2)c2ccc(S(=O)(=O)N(C)C)cc21 ; CN[C@H]1CC[C@@H](c2ccc(Cl)c(Cl)c2)c2ccc(CO)cc21]

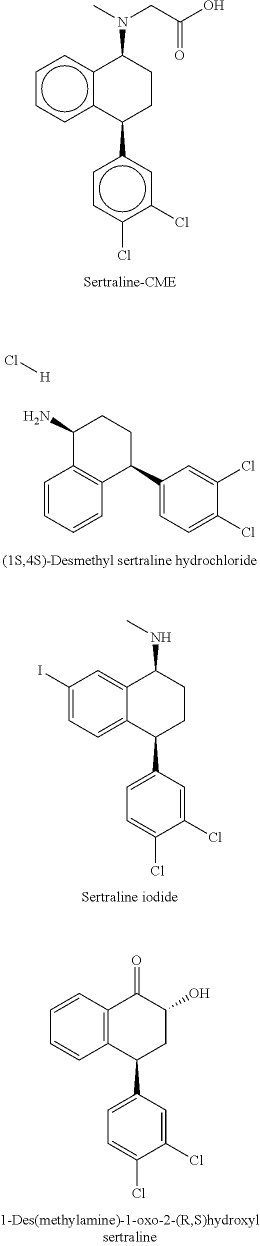

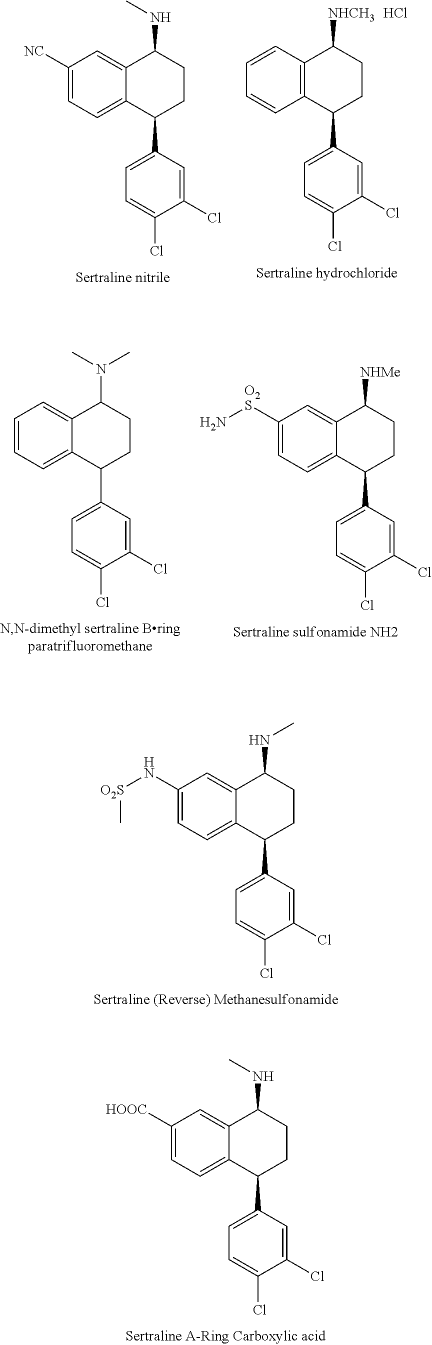

The term “serotonin-norepinephrine reuptake inhibitor” or “SNRI” refers to a family of compounds which are capable of inhibiting the reuptake of serotonin by blocking the serotonin transporter and the reuptake of norepinephrine by blocking the norepinephrine transporter. This family includes compounds such as, but not limited to, venlafaxine (CAS 93413-69-5), desvenlafaxine (CAS 93413-62-8), duloxetine (CAS 116539-59-4), milnacipran (CAS 92623-85-3), Sibutramine (106650-56-0), Tramadol (CAS 27203-92-5) and Bicifadine (CAS 71195-57-8). Any method known in the art can determine whether a given compound acts as a SNRI, these include but are not limited to, assaying the ability to reduce the uptake of serotonin and norepinephrine by brain synaptosomes as described essentially in Bolden-Watson C, Richelson E. (Life Sci. 1993; 52(12): 1023-9).

In one embodiment, the SNRIs are tricyclic antidepressants which are SNRIs having a general molecular structure comprising three rings Prominent among the tricyclic anti-depressants are the linear tricyclics, e.g., imipramine, desipramine, amitriptyline, nortriptyline, protriptyline, doxepin, ketipramine, mianserin, dothiepin, amoxapine, dibenzepin, melitracen, maprotiline, flupentixol, azaphen, tianeptine and related compounds showing similar activity. Angular tricyclics include indriline, clodazone, nomifensin, and related compounds. A variety of other structurally diverse anti-depressants, e.g., iprindole, wellbatrin, nialamide, milnacipran, phenelzine and tranylcypromine have been shown to produce similar activities. They are functionally equivalent to the tricyclic antidepressants and are therefore included within the scope of the invention. Thus, the term tricyclic anti-depressant is intended by the present inventor to embrace the broad class of anti-depressants described above together with related compounds sharing the common property that they all possess anti-depressant activity and which include, without being limited to, compounds such as amitriptyline, amitriptylinoxide, carbamazepine, butriptyline, clomipramine, demexiptiline, desipramine, dibenzepin, dimetacrine, dosulepin/dothiepin, Doxepin, Imipramine, Imipraminoxide, Iprindole, Lofepramine, Melitracen, Metapramine, Nitroxazepine, Nortriptyline, Noxiptiline, pregabalin, Propizepine, Protriptyline, Quinupramine and Trimipramine.

The term “noradrenaline reuptake inhibitor”, “NRI”, “NERI”, “adrenergic reuptake inhibitor” or “ARI” refers to a family of compounds which are capable of blocking reuptake of noradrenaline and adrenaline by blocking the action of the norepinephrine transporter (NET). This family of compounds includes the selective NRIs which block exclusively the NET without affecting other monoamine transporters as well as nonselective NRIs such as the SNRIs, which block the norepinephrine transporter and the serotonin transporter (see above), the norepinephrine-dopamine reuptake inhibitors (NDRI), which block the norepinephrine and the dopamine transporters (see below), triciclyc antidepressants and tetracyclic antidepressants (see above). Suitable selective NRIs adequalte for the present invention include, without being limited to, Atomoxetine/Tomoxetine (Strattera® or CAS 83015-26-3), Mazindol (Mazanor®, Sanorex® or CAS 22232-71-9), Reboxetine (Edronax®, Vestra® or CAS 98819-76-2) and Viloxazine (Vivalan® or CAS 46817-91-8).

The term “dopamine reuptake inhibitor” or “DRI” acts as a reuptake inhibitor for the neurotransmitter dopamine by blocking the action of the dopamine transporter (DAT). This in turn leads to increased extracellular concentrations of dopamine and therefore an increase in dopaminergic neurotransmission. Suitable DRIs include, without being limited to, pharmaceutical drugs such as amineptine, Benzatropine/Benztropine, Bupropion, dexmethylphenidate, Esketamine, Etybenzatropine/Ethybe, Ponalide, Fencamfamine, Fencamine, Ketamine, Lefetamine, Medifoxamine, Mesocarb, Methylphenidate, Nefopam, Nomifensine, Pipradrol, Prolintane, Pyrovalerone, Tiletamine and Tripelennamine; research chemicals such as altropane, amfonelic acid, benocyclidine, brasofensine, bromantane, DBL-583, dichloropane, diclofensine, Dieticyclidine, difluoropine, gacyclidine, GBR-12,935, indatraline, ioflupane, Iometopane, manifaxine, radafaxine, tametraline, tesofensine, troparil and vanoxerine. Suitable DRIs can be identified using any method known to one of skill in the art such as the determination of the capacity of the putative DRI in inhibiting high-affinity uptake of the dopamine by synaptosomal preparations prepared from rat corpus striatum carried out as described using methods published by Kula et al, (Life Sciences 34: 2567-2575, 1984).

The term “endocannabinoid reuptake inhibitor” or “eCBRI”, as used herein, refers to any compound which acts as a reuptake inhibitor for endocannabinoids by blocking the action of the endocannabinoids transporter. Compounds having this activity can be identified using the method described in Beltramo, M. et al. (Science, 1997, 277: 1094-1097) based on the ability of the putative endocannabinoid reuptake inhibitor to block uptake of anandamide by rat neurons and astrocytes and include, without limitation, AM404, arvanil and olvanil.

The term “adenosine reuptake inhibitor” or “AdoRI” refers to a compound which acts as a reuptake inhibitor for the purine nucleoside and neurotransmitter adenosine by blocking the action of one or more of the equilibrative nucleoside transporters (ENTs). This in turn leads to increased extracellular concentrations of adenosine and therefore an increase in adenosinergic neurotransmission. Compounds having AdoRI activity can be identified using an in vitro assay based on the ability of the putative AdoRI in inhibiting adenosine uptake by erythrocytes as well as in vivo assays based on the ability of the putative AdoRI of inhibiting the vasodilator effect of adenosine as well as of preventing adenosine-mediated promotion of the growth of collateral vessels, all of which can be carried out essentially as described in U.S. Pat. No. 6,984,642 incorporated herein by reference. Suitable AdoRI include, without being limited to, acadesine, acetate, Barbiturates, Benzodiazepines, Calcium Channel Blockers, Carbamazepine, Carisoprodol, Cilostazol, Cyclobenzaprine, Dilazep, Dipyridamole, Estradiol, Ethanol (Alcohol), Flumazenil, Hexobendine, Hydroxyzine, Indomethacin, Inosine, KF24345, Meprobamate, Nitrobenzylthioguanosine, Nitrobenzylthioinosine, Papaverine, Pentoxifylline, Phenothiazines, Phenytoin, Progesterone, Propentofylline, Propofol, Puromycin, R75231, RE 102 BS, Soluflazine, Toyocamycin, Tracazolate, Tricyclic Antidepressants.

The term “Excitatory Amino Acid Reuptake Inhibitor” or “EAARI”, refer to compounds which inhibit the reuptake of excitatory Amino Acid by blocking of the Excitatory Amino Acid transporter or EEATs. Many compounds are known to bind to EAATs and inhibit transporter function. Inhibitors of EAATs fall into two major classes that differ in their mode of action: non-transportable blockers and competitive substrates. Suitable EAARIs include, without being limited to, DL-threo-beta-Benzyloxyaspartate, kainite, dihydrokainate, 2S4R4MG, threo-P-hydroxyaspartate, L-trans-pyrrolidine-2,4-dicarboxylic acid (t-2,4-PDC) Suitable EEARIs can be identified for instance using the assay described by Shimamotot et al. (Molecular Pharmacology, 1998, 53: 195-201) based on the ability of the putative EEARI to inhibit uptake of radio labelled glutamate by Cos-1 cells expressing the human excitatory amino acid transporter-1 (EAAT1) or the human excitatory amino acid transporter-2 (EEAT2).

The term “glutamate reuptake inhibitor” or “GluRI”, refers to a compound which acts as a reuptake inhibitor for the glutamate by blocking the action of one or more of the glutamate transporters. Suitable inhibitors of glutamate reuptake encompass any one of those inhibitors that are already known in the art, including, illustratively, threo-3hydroxy-DL-aspartic acid (THA), (2S)-trans-pyrrolidine-2,4-dicarboxylic acid (PDC), aminocaproic acid, and (2S,3S)-3-{3-[4-(Trifluoromethyl)benzoylamino]benzyloxy} aspartate. Compounds having GluRI activity can be identified for instance using the assay described by Shimamotot et al. (Molecular Pharmacology, 1998, 53: 195-201) based on the ability of the putative GluRI to inhibit uptake of radio labelled glutamate into Cos-1 cells expressing the human excitatory amino acid transporter-1 (EAAT1) or the human excitatory amino acid transporter-2 (EEAT2).

The term “GABA reuptake inhibitor” or “GRI”, refers to a compound which acts as a reuptake inhibitor for the neurotransmitter gamma-aminobutyric acid (GABA) by blocking the action of the gamma-aminobutyric acid transporters (GATs). This in turn leads to increased extracellular concentrations of GABA and therefore an increase in GABAergic neurotransmission.

Suitable inhibitors of GABA reuptake include, without being limited to, adhyperforin (found in Hypericum perforatum (St. John's Wort)), CI-966, deramciclane (EGIS-3886), Guvacine (C10149), hyperforin (found in Hypericum perforatum (St. John's Wort)), Nipecotic acid, NNC 05-2090, NNC-71 1, SKF-89976A, SNAP-5114, stiripentol and Tiagabine (Gabitril) which are described in Borden LAEur J Pharmacol. 1994, 269: 219-224). Methods for detecting whether a given compound is a GABA reuptake inhibitor are known in the art and are described, e.g., in U.S. Pat. Nos. 6,906,177; 6,225,115; 4,383,999 and Ali, F. E., et al. (J. Med. Chem. 1985, 28, 653-660). These methods usually comprise contacting a cell with radio labelled GABA and detecting the uptake of the GABA in the presence and absence of a candidate compound.

The term “glycine reuptake inhibitor” or “GlyRI” refers to a compound which acts as a reuptake inhibitor for the neurotransmitter glycine by blocking the action of the glycine transporters (GlyTs) including compounds which block the glycine transporter (type 1) GlyTI which is involved in removing of glycine from the synaptic cleft as well as GlyT2, which is required for the reuptake and reloading of glycine into the synaptic vesicle (Gomeza et al, (2003) Curr Opin Drug Discov Devel 6(5): 675-82). Suitable glycine reuptake inhibitors for use in the present invention include GlyT1-specific inhibitors such as, but not limited to, N-methyl-N-[[(1R,2S)-1,2,3,4-tetrahydro-6-methoxy-1-phenyl-2-naphthalenyljmethyl glycine (the free base of MTHMPNMglycine), 4-[3-fluoro-4-propoxyphenyl]-spiro[2H-1-benzopyran-2,4′-piperidine]-1′-acetic acid (the free base of FPPSBPAA) which are described in PCT publication WO/0007978 and WO/0136423, ALX 5407, sarcosine, 5,5-diaryl-2-amino-4-pentenoates or the compounds described in PCT publication WO/0208216 as well as GlyT2-specific inhibitors such as those described in PCT publication WO/05044810A, which contents are incorporated by reference in their entirety. Methods for detecting GlyT1-specific or GlyT2-specific reuptake inhibitors are known in the art and include, for example, the method described in PCT publication Nos. WO/05018676A or WO/05044810 wherein cells expressing the relevant receptor (GlyT1 or GlyT2) are contacted with radio labelled glycine in the presence of the compound which reuptake inhibitory activity is to be tested and the amount of glycine which is found inside the cell after a given time is determined.

The term “norepinephrine-dopamine reuptake inhibitor” or “NDRI”, as used herein, refers to a compound which acts as a reuptake inhibitor for the neurotransmitters norepinephrine and dopamine by blocking the action of the norepinephrine transporter (NET) and the dopamine transporter (DAT), respectively. This in turn leads to increased extracellular concentrations of both norepinephrine and dopamine and therefore an increase in adrenergic and dopaminergic neurotransmission. Suitable NDRIs for use in the conjugates of the present invention include, without being limited to, Amineptine (Survector®, Maneon®, Directin®), Bupropion (Wellbutrin®, Zyban®), Dexmethylphenidate (Focalin®), Fencamfamine (Glucoenergan®, Reactivan®), Fencamine (Altimina®, Sicoclor®), Lefetamine (Santenol®), Methylphenidate (Ritalin®, Concerta®), Nomifensine (Merital®), Pipradrol (Meretran®), Prolintane (Promotil®, Katovit®), Pyrovalerone (Centroton®, Thymergix®), Nefopam (Acupan®), adhyperforin (found in Hypericum perforatum (St. John's Wort)), hyperforin (found in Hypericum perforatum (St. John's Wort)), Cocaine, Desoxypipradrol (2-DPMP), Diphenylprolinol (D2PM), Methylenedioxypyrovalerone (MDPV), Cilobamine, Manifaxine (GW-320,659), Radafaxine (GW-353,162), Tametraline (CP-24,441).

According to one embodiment, the conjugate of the invention comprises a cell-targeting moiety that binds specifically to a neurotransmitter transporter which is a selective serotonin reuptake inhibitor (SSRI).

According to a specific embodiment, the conjugate of the invention comprises a SSRI selected from the group consisting of sertraline, a sertraline-structural analog, fluoxetine, fluvoxamine, paroxetine, indapline, zimelidine, citalopram, dapoxetine, escitalopram, and mixtures thereof.

According to a specific embodiment, the conjugate of the invention comprises the SSRI sertraline or a structural analog thereof as defined above.

According to a specific embodiment, when the cell-targeting moiety targets a bone cell, the targeting moiety can include synthetic components such as a tetracycline or a bisphosphonate (BP).

The synthetic miR-135 molecule and the cell-targeting moiety may be coupled directly or indirectly via an intervening moiety or moieties, such as a linker, a bridge, or a spacer moiety or moieties.

According to one embodiment, the synthetic miR-135 molecule and the cell-targeting moiety may be directly coupled. Alternatively, according to another embodiment, both moieties may be linked by a connecting group.

The terms “connecting group”, “linker”, “linking group” and grammatical equivalents thereof are used herein to refer to an organic moiety that connects two parts of a compound. The cell-targeting moiety can be attached to any nucleotide in the sense (e.g. guide) or antisense (e.g. passenger) strand within the miR-135 molecule, but it can be preferably coupled through the 3′ terminal nucleotide and/or 5′ terminal nucleotide. An internal conjugate may be attached directly or indirectly through a linker to a nucleotide at a 2′ position of the ribose group, or to another suitable position.

As mentioned, the synthetic miR-135 molecule of the invention is preferably a double-stranded nucleic acid molecule, therefore the conjugate moiety (i.e. cell-targeting moiety) can be attached to the 3′ terminal nucleotide of the guide sequence, the 5′ terminal nucleotide of the guide sequence, the 3′ terminal nucleotide of the passenger sequence, and/or the 5′ terminal nucleotide of the passenger sequence.

Though not wishing to be limited by definitions or conventions, in this application the length of the linker is described by counting the number atoms that represent the shortest distance between the atom that joins the conjugate moiety (i.e. cell-targeting moiety) to the linker and the oxygen atom of the terminal phosphate moiety associated with the miR-135 oligonucleotide through which the linker is attached to the miR-135 oligonucleotide. In cases where the linker comprises one or more ring structures, counting the atoms around the ring that represent the shortest path is preferred.

Suitable linker groups for use in the present invention include, without being limited to, modified or unmodified nucleotides, nucleosides, polymers, sugars, carbohydrates, polyalkylenes such as polyethylene glycols and polypropylene glycols, polyalcohols, polypropylenes, mixtures of ethylene and propylene glycols, polyalkylamines, polyamines such as polylysin and spermidine, polyesters such as poly(ethyl acrylate), polyphosphodiesters, aliphatics, and alkylenes. Moreover, linkers/linker chemistries that are based on omega-amino-1,3-diols, omega-amino-1,2-diols, hydroxyprolinols, omega-amino-alkanols, diethanolamines, omega-hydroxy-1,3-diols, omega-hydroxy-1,2-diols, omega-thio-1,3-diols, omega-thio-1,2-diols, omega-carboxy-1,3-diols, omega-carboxy-1,2-diols, co-hydroxy-alkanols, omega-thio-alkanols, omega-carboxy-alkanols, functionalized oligoethylene glycols, allyl amine, acrylic acid, allyl alcohol, propargyl amine, propargyl alcohol, and more, can be applied in this context to generate linkers of the appropriate length.

The linker may also confer other desirable properties such as improved aqueous solubility, optimal distance of separation between the conjugate moiety (i.e. cell-targeting moiety) and the miR-135 molecule, flexibility (or lack thereof), specific orientation, branching, and others.

According to one embodiment, the connecting group has the following structure:

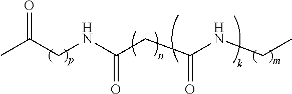

wherein
m, n and p are selected from 0, 1, 2, 3, 4, 5, 6, 7, 8, 9, 10, 11, 12 and 13,
wherein the sum of m+n+p is an integer number selected from 7, 8, 9, 10, 11, 12, 13, 14, 15, 16, 17 and 18 and
wherein k is 0 or 1.

According to one embodiment, p is 5, n is 2, k is 1 and m is 6 giving a linker having the structure:

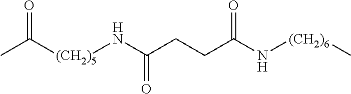

According to one embodiment, p is 5, n and k are 0 and m is 6 giving a linker having the structure:

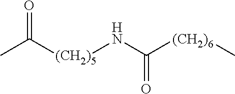

According to one embodiment, the linker comprises more than one coupling for the cell-targeting moiety. In a preferred embodiment, the linker is a bivalent or trivalent linker, i.e. 2 or 3 molecules of agent can be coupled, respectively.

In the case wherein more than one molecule of cell-targeting moiety are coupled to the miR-135 nucleic acid through a linker, the molecules can represent the same or different cell-targeting moiety.

According to one embodiment, the bivalent or trivalent linker has the following formula:

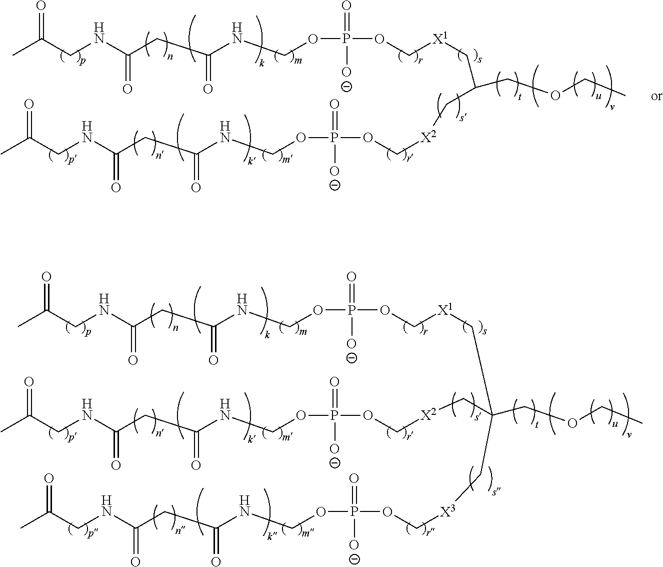

m, m′, m″, n, n′, n″, p, p′, p″, r, r′, r″, s, s′, s″, t and u are independently selected from O, 1, 2, 3, 4, 5, 6, 7, 8, 9, 10, 11, 12 and 13;
k, k′, k″ and v are independently selected from 0 and 1; and
X¹, X² and X³ are independently selected from CH₂, O, S, NH, CO, C(O)O and C(O)NH.

Depending on the values of the above mentioned groups, branched linkers can be symmetrical or asymmetrical.

In a specific embodiment, the linker is a bivalent linker as shown above wherein p and p′ are 5, n and n′ are 2, k and k′ are 1 and m and m′ are 6. In a specific embodiment, the linker is a bivalent linker wherein p and p′ are 5, n, n′, k and k′ are 0 and m and m′ are 6.

In a specific embodiment, the linker is a bivalent linker as shown above wherein r and r′ are 4, s and s′ are 1, t and v are 0 and X¹ and X² represent C(O)NH. In another embodiment, the linker is a bivalent linker wherein r is 2, r′ is 0, s is 1, s′ is 0, t and v are 0 and X¹ and X² represent CH₂.

In a specific embodiment, the linker is a bivalent linker wherein p and p′ are 5, n and n′ are 2, k and k′ are 1, m and m′ are 6, r and r′ are 4, s and s′ are 1, t and v are 0 and X¹ and X² represent C(O)NH.

In another embodiment, the linker is a bivalent linker wherein p and p′ are 5, n and n′ are 2, k and k′ are 1, m and m′ are 6, r is 2, r′ is 0, s is 1, s′ is 0, t and v are 0 and X¹ and X² represent CH₂.

In another embodiment, the linker is a bivalent linker wherein p and p′ are 5, n, n′, k and k′ are 0 and m and m′ are 6, r and r′ are 4, s and s′ are 1, t and v are 0 and X¹ and X² represent C(O)NH.

In another embodiment, the linker is a bivalent linker wherein p and p′ are 5, n, n′, k and k′ are 0 and m and m′ are 6, r is 2, r′ is 0, s is 1, s′ is 0, t and v are 0 and X¹ and X² represent CH₂.

In a specific embodiment, the linker is a trivalent linker as shown above wherein p, p′ and p″ are 5, n, n′ and n″ are 2, k, k′ and k″ are 1 and m, m′ and m″ are 6. In a specific embodiment, the linker is a trivalent linker wherein p, p′ and p″ are 5, n, n′, n″, k, k′ and k″ are 0 and m, m′ and m″ are 6.

In a specific embodiment, the linker is a trivalent linker as shown above wherein r, r′ and r″ are 3, s, s′ and s″ are 1, t is 1, v is 0 and X¹, X² and X³ represent O. In another embodiment, the linker is a trivalent linker wherein r, r′ and r″ are 3, s, s′ and s″ are 1, t is 1, u is 3, v is 1 and X¹, X² and X³ represent O.

In a specific embodiment, the linker is a trivalent linker wherein p, p′ and p″ are 5, n, n′ and n″ are 2, k, k′ and k″ are 1, m, m′ and m″ are 6, r, r′ and r″ are 3, s, s′ and s″ are 1, t is 1, v is 0 and X¹, X² and X³ represent O.

In another embodiment, the linker is a trivalent linker wherein p, p′ and p″ are 5, n, n′ and n″ are 2, k, k′ and k″ are 1, m, m′ and m″ are 6, r, r′ and r″ are 3, s, s′ and s″ are 1, t is 1, u is 3, v is 1 and X¹, X² and X³ represent O.

In another embodiment, the linker is a trivalent linker wherein p, p′ and p″ are 5, n, n′, n″, k, k′ and k″ are 0, m, m′ and m″ are 6, r, r′ and r″ are 3, s, s′ and s″ are 1, t is 1, v is 0 and X¹, X² and X³ represent O.

In another embodiment, the linker is a trivalent linker wherein p, p′ and p″ are 5, n, n′, n″, k, k′ and k″ are 0, m, m′ and m″ are 6, r, r′ and r″ are 3, s, s′ and s″ are 1, t is 1, u is 3, v is 1 and X¹, X² and X³ represent O.

According to one embodiment, the linking compound is selected from a phosphodiester, a phosphorothioate, a carbamate, a methylphosphonate, a guanidinium, a sulfamate, a sulfamide, a formacetal, a thioformacetal, a sulfone, an amide and mixtures thereof.

According to one embodiment, the linking compound is a C10 N-hydroxysuccimide ester linker (i.e. a C10 linker).

According to a specific embodiment, the conjugate of the invention has the structure:

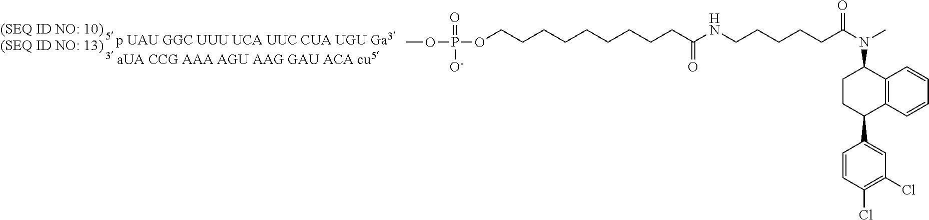 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to a specific embodiment, the conjugate of the invention has the structure:

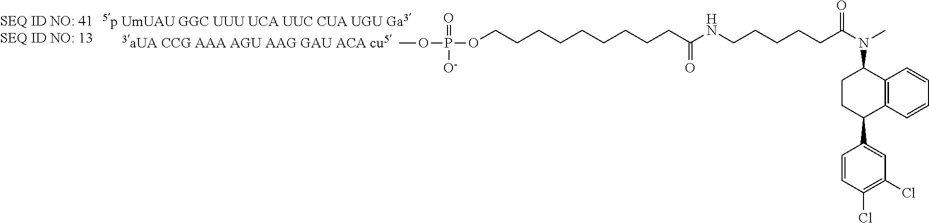 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to a specific embodiment, the conjugate of the invention has the structure:

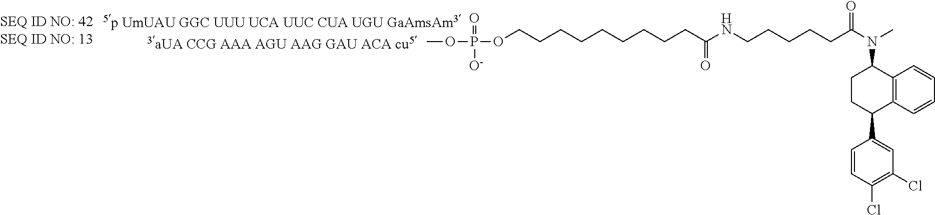 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to a specific embodiment, the conjugate of the invention has the structure:

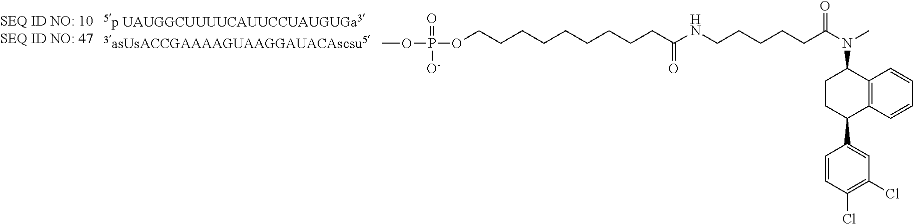 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to a specific embodiment, the conjugate of the invention has the structure:

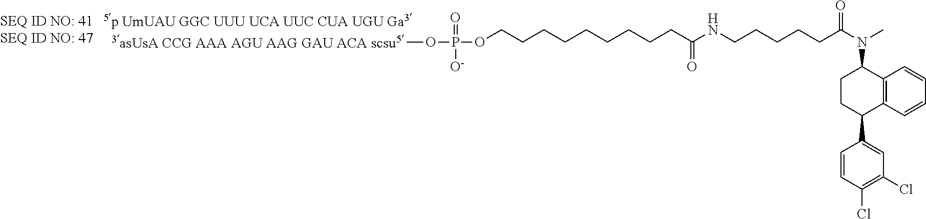 [SMILES: COP(=O)([O-])OCCCCCCCCCC(=O)NCCCCCC(=O)N(C)[C@@H]1CC[C@H](c2ccc(Cl)c(Cl)c2)c2ccccc21]

According to a specific embodiment, the conjugate of the invention has the structure:

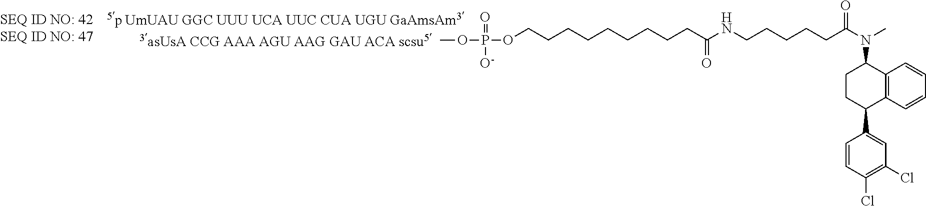 [SMILES: COP(=O)([O-])OCCCCCCCCCC(=O)NCCCCCC(=O)N(C)[C@@H]1CC[C@H](c2ccc(Cl)c(Cl)c2)c2ccccc21]

The conjugates of the invention can be prepared using techniques known by those skilled in the art. The synthesis of conjugates may involve the selective protection and deprotection of functional groups. Suitable protecting groups are well known for the skilled person in the art. For example, a general review of protecting groups in organic chemistry is provided by Wuts, P. G. M. and Greene T. W. in Protecting Groups in Organic Synthesis (4^{th}, Ed. Wiley-Interscience), and by Kocienski P. J. in Protecting Groups (3^{rd} Ed. Georg Thieme Verlag).

For purposes of the present invention, “protecting group” shall be understood to mean chemical modifications, which have been incorporated at either terminus of the miR-135 oligonucleotide. Non-limiting examples of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; amino hexyl phosphate; 3′-phosphate; 3′-phosphorothioate; phosphorodithioate; or bridging or non-bridging methylphosphonate moiety. Details are described in WO97/26270, incorporated by reference herein. The 3′-cap includes, for example, 4′,5′-methylene nucleotide; 1-(beta-D-erythrofuranosyl) nucleotide: 4′-thio nucleotide, carbocyclic nucleotide; 5′-amino-alkyl phosphate; 1,3-diamino-2-propyl phosphate, 3-aminopropyl phosphate; 6-amino hexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inveiled abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties. See also Beaucage and Iyer, 1993, Tetrahedron 49, 1925; the contents of which are incorporated by reference herein.

According to one embodiment, the synthetic miR-135 molecule or conjugate of the invention may be further modified to enhance the activity, cellular distribution or cellular uptake of the miR-135 nucleic acid by chemically linking to the nucleic acid of miR-135 or to the protecting group one or more moieties or conjugates.

According to one embodiment, the synthetic miR-135 molecule or conjugate of some embodiments of the invention further comprises at least one cell-penetrating moiety. Such moieties include but are not limited to lipidic moieties (i.e. naturally occurring or synthetically produced lipids) such as a cholesterol moiety (Letsinger et al, Proc. Natl. Acid. Sci. USA, 199, 86, 6553-6556), cholic acid (Manoharan et al, Biorg. Med. Chem. Let., 1994 4 1053-1060), a thioether, e.g., beryl-S-tritylthiol (Manoharan et al, Ann. N.Y. Acad. Sci., 1992, 660, 306-309; Manoharan et al, Biorg. Med. Chem. Let., 1993, 3, 2765-2770), a thiocholesterol (Oberhauser et al, Nucl. Acids Res., 1992, 20, 533-538), an aliphatic chain, e.g., dodecandiol or undecyl residues (Saison-Behmoaras et al, EMBO J, 1991, 10, 11 11-1118; Kabanov et al, FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al, Tetrahedron Lett., 1995, 36, 3651-3654; Shea et al, Nucl. Acids Res., 1990, 18, 3777-3783), a polyamine or a polyethylene glycol chain (Manoharan et al., Nucleosides and Nucleotides, 1995, 14, 969-973), or adamantane acetic acid (Manoharan et al., Tetrahedron Lett., 1995, 36, 3651-3654), a palmityl moiety (Mishra et al, Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Additional lipid moieties which may be used in accordance with the present invention include, but are not limited to, fatty acids; fats; oils; waxes; cholesterol; sterols; fat-soluble vitamins, such as vitamins A, D, E and K; monoglycerides; diglycerides, and phospholipids. According to one embodiment, fatty acids include, for example, lauroic acid (C12), myristic acid (C14), palmitic acid (C16), stearic acid (C18), docosanoic acid (C22), and hybrid of lithocholic acid and oleylamine (lithocholic-oleyamine, C43).

According to a specific embodiment, the lipid moiety is palmitoyl.

According to a specific embodiment, the lipid moiety is cholesteryl.

According to one embodiment, the lipid moiety is C18-C18 (i.e. C18-phosphodiester-C18), e.g. wherein the C18 is provided as a phosphoramidite and is added to the 5′ end of the oligonucleotide by a coupling reaction, and then a second C18 is attached to the previous one following the same coupling reaction.

According to one embodiment, the cell-penetrating moiety is a peptide or protein.

According to a specific embodiment, the cell-penetrating moiety is an oxytocin peptide or a compound derived therefrom (e.g. recombinant or synthetic oxytocin).

According to a specific embodiment, the cell-penetrating moiety is a human seric albumin or other plasma protein or a partial peptide thereof.

According to a specific embodiment, the cell-penetrating moiety is a peptide-shuttle.

Exemplary peptide shuttles include, but are not limited to, Angiopep-2, RGV29, and THR.

According to one embodiment, the cell-penetrating moiety is a small drug. Exemplary small drugs which can be used to target e.g. 5-HT neurons and post-synaptic neurons, include but are not limited to, ligands for 5-HT1a receptor [11C] DASB, [11C] WAY100635 or [18F] MPPF.

Alternatively, the moiety capable of enhancing cellular distribution may be a low molecular weight compound or polypeptide which is capable of being specifically translocated across biological barriers by the use of receptor-mediated endocytosis using specific transporters present in the biological barriers. A wide array of uptake receptors and carriers, with an even wider number of receptor-specific ligands, are known in the art. Preferred ligands for receptors that mediates endocytosis and/or transcytosis for use in accordance with present invention include e.g. ligands for, or that specifically bind to the thiamine transporter, folate receptor, vitamin B12 receptors, asialoglycoprotein receptors, alpha(2,3)-sialoglycoprotein receptor (with e.g., the FC5 and FC44 nanobodies consisting of llama single-domain antibodies (sdAbs) as receptor-specific ligands), transferrin-1 and -2 receptors, scavenger receptors (class A or B, types I, II or III, or CD36 or CD 163), low-density lipoprotein (LDL) receptor, LDL-related protein 1 receptor (LRP1, type B), the LRP2 receptor (also known as megalin or glycoprotein 330), diphtheria toxin receptor (DTR, which is the membrane-bound precursor of heparin-binding epidermal growth factor-like growth factor (HB-EGF)), insulin receptor, insulin-like growth factors (IGF) receptors, leptin receptors, substance P receptor, glutathione receptor, glutamate receptors and mannose 6-phosphate receptor.

Exemplary ligands that bind to these receptors for use in accordance with the present invention include e.g. ligands selected from the group consisting of: lipoprotein lipase (LPL), alpha2-macroglobulin (alpha2M), receptor associated protein (RAP), lactoferrin, desmoteplase, tissue- and urokinase-type plasminogen activator (tPA/uPA), plasminogen activator inhibitor (PAI-I), tPA/uPA:PAI-1 complexes, melanotransferrin (or P97), thrombospondin 1 and 2, hepatic lipase, factor V11a/tissue-factor pathway inhibitor (TFPI), factor VIIIa, factor IXa, Abeta1-40, amyloid-beta precursor protein (APP), C1 inhibitor, complement C3, apolipoproteinE (apoE), Pseudomonas exotoxin A, CRM66, HIV-I Tat protein, rhinovirus, matrix metalloproteinase 9 (MMP-9), MMP-13 (collagenase-3), spingolipid activator protein (SAP), pregnancy zone protein, antithrombin III, heparin cofactor II, alpha1-antitrypsin, heat shock protein 96 (HSP-96), platelet-derived growth factor (PDGF), apolipoproteinJ (apoJ, or clusterin), ABETA bound to apoJ and apoE, aprotinin, angio-pepl, very-low-density lipoprotein (VLDL), transferrin, insulin, leptin, an insulin-like growth factor, epidermal growth factors, lectins, peptidomimetic and/or humanized monoclonal antibodies or peptides specific for the receptors, hemoglobin, non-toxic portion of a diphtheria toxin polypeptide chain, all or a portion of the diphtheria toxin B chain (including DTB-His (as described by Spilsberg et al., 2005, Toxicon., 46(8):900-6)), all or a portion of a non-toxic mutant of diphtheria toxin CRM197, apolipoprotein B, apolipoprotein E (e.g., after binding to polysorb-80 coating on nanoparticles), vitamin D-binding protein, vitamin A/retinol-binding protein, vitamin B12/cobalamin plasma carrier protein, glutathione and transcobalamin-B12.

According to a specific embodiment, the conjugate of the invention further comprises a group that facilitates the transport of the conjugate across biological membranes. According to one embodiment, the group is amphipathic. Exemplary agents include, without being limited to, penetratin, the fragment of the Tat protein comprising amino acids 48-60, the signal sequence based peptide, PVEC, transportan, amphiphilic model peptide, Arg9, bacterial cell wall permeating peptide, LL-37, cecropin P1, α-defensin, β-defensin, bactenectin, PR-39 and indolicidin. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

According to one embodiment, the synthetic miR-135 molecule or conjugate of some embodiments of the invention further comprise at least one moiety for transport across the BBB.

According to one embodiment, the moiety for transport across the BBB is a peptide or protein.

According to one embodiment, the moiety for transport across the BBB is a neurotropic or neurotoxin derived peptide or a variant thereof.

Exemplary peptides include, but are not limited to, EPO-fusion protein (e.g. fused to a peptidomimetic antibody with an affinity to human insulin receptor, anti-transferrin receptor (TfR) monoclonal antibody, rabis virus glycoprotein (e.g. chimeric RVG fragment peptides), as discussed in Razzak et al. Int. J. Mol. Sci. (2019), 20: 3108, incorporated herein by reference.

According to one embodiment, the moiety for transport across the BBB is a BBB-shuttle (also referred to as a Trojan horse antibody). Exemplary BBB-shuttles include, but are not limited to, Angiopep-2, DAngiopep-2, ApoB, ApoE, THR, THRre, RVG29, TGN, ^{D}CDX, Apamin, TGN and TAT (47-57). Additional BBB-shuttles are discussed in Oller-Salvia et al., Chem. Soc. Rev. (The Royal Society of Chemistry) 2016, incorporated herein by reference.

In another specific embodiment of the invention, the conjugate of the invention may further comprise an endosomolytic ligand. Endosomolytic ligands promote the lysis of the endosome and/or transport of the composition of the invention, or its components, from the endosome to the cytoplasm of the cell. The endosomolytic ligand may be a polyanionic peptide or peptidomimetic which shows pH-dependent membrane activity and fusogenicity. In certain embodiments, the endosomolytic ligand assumes its active conformation at endosomal pH. Exemplary endosomolytic ligands include e.g. the GAL4 peptide (Subbarao et al., Biochemistry, 1987, 26: 2964-2972), the EALA peptide (Vogel et al., J. Am. Chem. Soc, 1996, 118: 1581-1586), and their derivatives (Turk et al., Biochem. Biophys. Acta, 2002, 1559: 56-68), the INF-7 peptide, the Inf HA-2 peptide, the diINF-7 peptide, the diINF3 peptide, the GLF peptide, the GALA-INF3 peptide and the INF-5 peptide.

Any of the above described ligands or moieties (e.g. cell-penetrating moiety or the moiety for transport across the BBB) may be selected by the skilled person taking into consideration the target tissue, the target cell, the administration route, the pathway that the oligonucleotide is expected to follow, etc.

According to one embodiment, when the ligand or moiety (e.g. cell-penetrating moiety or the moiety for transport across the BBB) is a peptide or peptide product, it may be subjected to in-vitro modification (e.g., PEGylation, lipid modification, etc.) so as to confer the peptide's amino acid sequence with stability (e.g., against protease activities) and/or solubility (e.g., within a biological fluid such as blood, digestive fluid) while preserving its biological activity and prolonging its half-life.

The conjugates of the invention are typically synthesized using standard procedures in organic synthesis. The skilled person will appreciate that the exact steps of the synthesis will depend on the exact structure of the conjugate which has to be synthesized. For instance, if the conjugate comprises a single nucleic acid strand conjugated to the cell-targeting moiety through its 5′ end, then the synthesis is usually carried out by contacting an amino-activated oligonucleotide and a reactive activated cell-targeting moiety.

According to one embodiment, when the conjugate comprises a double stranded miR-135 nucleic acid, then the sense (e.g. guide) and antisense (e.g. passenger) strands are synthesized separately and annealed in vitro using standard molecular biology procedures (as discussed in detail hereinabove). In a typical conjugate, the first nucleic acid strand carries the cell-targeting moiety and the second nucleic acid strands carries a protecting group.

In one embodiment, the cell-targeting moiety is coupled to the 5′ end of the first nucleic acid strand and/or the protecting group is attached to the 5′ end of the second nucleic acid strand, although the attachment of the cell-targeting moiety or of the protecting group can also be carried out at the 3′ ends of the nucleic acid strands.

In a specific embodiment, the cell-targeting moiety is coupled to the 5′ end of the passenger strand and/or the protecting group is attached to the 5′ end of the guide strand.

It will be appreciated that when the cell-targeting moiety is coupled to the 5′ end or to the 3′ end of the sense (e.g. guide) or antisense (e.g. passenger) strand of the synthetic miR-135 molecule, the cell-penetrating moiety and/or the moiety for transport across the BBB may be coupled to any of the remaining ends (5′ or 3′) of the double stranded molecule.

According to one embodiment, the cell-penetrating moiety and/or the moiety for transport across the BBB may be coupled to the cell-targeting moiety.

According to a specific embodiment, the lipidic moiety (e.g. cholesterol) is coupled to the cell-targeting moiety (e.g. SSRI such as sertraline).

According to one embodiment, when the synthetic miR-135 molecule is not conjugated to a cell-targeting moiety, the cell-penetrating moiety and/or the moiety for transport across the BBB may be coupled to any of the ends (5′ or 3′) of the sense (e.g. guide) or antisense (e.g. passenger) strand of the synthetic miR-135 molecule.

According to one embodiment, the synthetic miR-135 molecule or conjugate of some embodiments of the invention and the cell-penetrating moiety and/or the moiety for transport across the BBB may be directly coupled. Alternatively, according to another embodiment, both moieties may be linked by a connecting group.

Suitable linker groups for use in the present invention are discussed above.

According to a specific embodiment, the linker is a palmitoyl modified linker having the following structure (wherein ASO is the oligonucleotide):

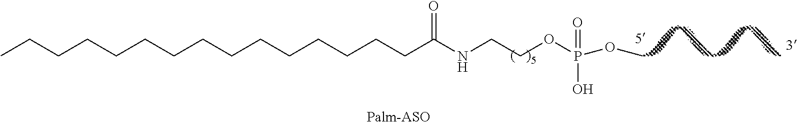

According to a specific embodiment, the linker is a phosphodiester unit (e.g. phosphodiester (PO)-trinucleotide linker and a hexylamino spacer) to conjugate palmitate to the oligonucleotide.

According to a specific embodiment, the linker is a cholesterol linker. Exemplary linkers include the triethyl glycol [TEG] linker and 2-aminobutyl-1-3-propanediol [C7] linker.

According to a specific embodiment, the conjugates of the invention are synthesized using the following steps:
(i) Activating the cell-penetrating moiety (e.g. which binds specifically to a neurotransmitter transporter). According to a specific embodiment, the activation group in the agent is a succinimide or an amino group,
(ii) Activating the passenger strand (or the guide strand) on its 5′ end. According to a specific embodiment, the activation group in the oligonucleotide is amino group (wherein the agent has been activated by a succinimide group) or a carboxyl group (wherein the agent has been activated by an amine group),
(iii) Contacting the activated cell-penetrating moiety with the activated passenger strand (or the activated guide strand) under conditions adequate for the reaction between the two activation groups.

According to one embodiment, the following step may further be carried out:
(iv) Adding a protecting group to the guide strand (or the passenger strand). This step may be carried out using an oligonucleotide which reactive groups are blocked by acetylation or benzylation (the furanose groups), 2-cyanoethylation (the phosphodiester linkages) and FMOC (the exocyclic amino groups).

According to one embodiment, the following step may further be carried out:
(v) Annealing the guide and passenger strands.

According to one embodiment, and as mentioned above, in cases where the synthetic miR-135 molecule does not comprise a chemical modification it may be administered to the target cell (e.g. brain cells such as neuroglia cells, oligodendrocytes, choroid plexus (CP) cells, stem cells or differentiated stem cells) as part of an expression construct. In this case, the synthetic miR-135 molecule is ligated in a nucleic acid construct under the control of a cis-acting regulatory element (e.g. promoter) capable of directing an expression of the microRNA in the target cells (e.g. brain cells such as neuroglia cells, oligodendrocytes, CP cells, stem cells or differentiated stem cells) in a constitutive or inducible manner.

The expression constructs of the present invention may also include additional sequences which render it suitable for replication and integration in eukaryotes (e.g., shuttle vectors). Typical cloning vectors contain transcription and translation initiation sequences (e.g., promoters, enhances) and transcription and translation terminators (e.g., polyadenylation signals). The expression constructs of the present invention can further include an enhancer, which can be adjacent or distant to the promoter sequence and can function in up regulating the transcription therefrom. Polyadenylation sequences can also be added to the expression constructs of the present invention in order to increase the efficiency of expression.

In addition to the embodiments already described, the expression constructs of the present invention may typically contain other specialized elements intended to increase the level of expression of cloned nucleic acids or to facilitate the identification of cells that carry the recombinant DNA. The expression constructs of the present invention may or may not include a eukaryotic replicon.

The nucleic acid construct may be introduced into the target cells (e.g. brain cells such as neuroglia cells) of the present invention using an appropriate gene delivery vehicle/method (transfection, transduction, etc.) and an appropriate expression system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In order to circumvent the blood brain barrier, the constructs of the present invention may be administered directly into the brain (via the ventricle), into the olfactory bulb (via an intranasal administration), via the spinal cord (e.g. by an epidural catheter) or by expression in the choroid plexus, as further detailed herein. Additional modes of administration are discussed in detail herein below.

Additionally or alternatively, lipid-based systems may be used for the delivery of constructs or conjugated miR-135 molecules into the target cells (e.g. brain cells such as neuroglia cells) of the present invention.

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D., Liposomes Technology Inc., Elsevier, 1993, 63-105. (chapter 3); Winterhalter M, Lasic D D, Chem Phys Lipids, 1993 September; 64(1-3):35-43]. Any method known in the art can be used to incorporate micro-RNA (e.g. synthetic miR-135 molecule) into a liposome. For example, the micro-RNA polynucleotide agent (e.g. synthetic miR-135 molecule) may be encapsulated within the liposome. Alternatively, it may be adsorbed on the liposome's surface. Other methods that may be used to incorporate a pharmaceutical agent into a liposome of the present invention are those described by Alfonso et al., [The science and practice of pharmacy, Mack Publishing, Easton Pa 19^{th} ed., (1995)] and those described by Kulkarni et al., [J. Microencapsul. 1995, 12 (3) 229-46].

The liposomes used in the methods of the present invention may cross the blood barriers.

Thus, according to an embodiment the liposomes of the present invention do not comprise a blood barrier targeting polysaccharide (e.g. mannose) in their membrane portion. In order to determine liposomes that are especially suitable in accordance with the present invention a screening assay can be performed such as the assays described in U.S. Pat. Appl. No. 20040266734 and U.S. Pat. Appl. No. 20040266734; and in Danenberg et al., Journal of cardiovascular pharmacology 2003, 42:671-9; Circulation 2002, 106:599-605; Circulation 2003, 108:2798-804.

According to one embodiment, dual-targeted liposomes are utilized. Exemplary dual-targeted liposomes include, but are not limited to, Angiopep-2-oligoarginine, T7-TAT, THR-transportan, Tf-TAT, Tf-penetratin or Tf-mastoparan.

Additionally or alternatively, non-lipid based vesicles can be used according to this aspect of the present invention including exosomes, e.g. modified exosomes such as EVOX. Such exosomes can cross the BBB and release the therapeutic composition into the brain, e.g. brain cerebrospinal fluid (CSF).

Other non-lipid based vesicles that can be used according to this aspect of the present invention include but are not limited to polylysine, dendrimers and Gagomers.

Regardless of the method or construct employed, there is provided an isolated cell comprising the nucleic acid construct encoding the synthetic miR-135 molecule, as detailed above, or comprising the synthetic miR-135 molecule or conjugated form thereof.

According to a specific embodiment, the cell is a neuroglia cell (i.e. a neuron or a glial cell e.g., oligodendrocytes or astrocyte).

According to a specific embodiment, the neuroglia cell is a neuron such as a serotonergic neuron.

According to a specific embodiment, the cell is a cancerous cell.

According to a specific embodiment, the cell is a bone cell.

According to a specific embodiment, the cell is a muscle cell.

According to a specific embodiment, the cell is a gastrointestinal cell.

The synthetic miR-135 molecules of the invention are to be provided to the cells i.e., target cells (e.g. neuroglia cells) of the present invention in vivo (i.e., inside the organism or the subject) or ex vivo (e.g., in a tissue culture). In case the cells are treated ex vivo, the method preferably includes a step of administering such cells back to the individual (ex vivo cell therapy).

For ex vivo therapy, cells (e.g. brain cells such as neuroglia cells e.g. oligodendrocytes, CP cells, stem cells and/or differentiated stem cells) are preferably treated with the composition of the present invention (e.g., synthetic miR-135 molecule or conjugated form thereof), following which they are administered to the subject in need thereof.

Administration of the ex vivo treated cells of the present invention can be effected using any suitable route of introduction, such as intravenous, intraperitoneal, intra-kidney, intra-gastrointestinal track, subcutaneous, transcutaneous, intramuscular, intracutaneous, intrathecal, epidural, and rectal (as further discussed herein below). According to presently preferred embodiments, the ex vivo treated cells of the present invention may be introduced to the individual using intravenous, intra-kidney, intra-gastrointestinal track, and/or intraperitoneal administration.

The cells of the present invention (e.g. neuroglia cells such as oligodendrocytes, CP cells, stem cells, differentiated stem cells and/or cardiac cells) can be derived from either autologous sources or from allogeneic sources such as human cadavers or donors. Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

Encapsulation techniques are generally classified as microencapsulation, involving small spherical vehicles, and macroencapsulation, involving larger flat-sheet and hollow-fiber membranes (Uludag, H. et al. (2000). Technology of mammalian cell encapsulation. Adv Drug Deliv Rev 42, 29-64).

Methods of preparing microcapsules are known in the art and include for example those disclosed in: Lu, M. Z. et al. (2000). Cell encapsulation with alginate and alpha-phenoxycinnamylidene-acetylated poly(allylamine). Biotechnol Bioeng 70, 479-483; Chang, T. M. and Prakash, S. (2001) Procedures for microencapsulation of enzymes, cells and genetically engineered microorganisms. Mol Biotechnol 17, 249-260; and Lu, M. Z., et al. (2000). A novel cell encapsulation method using photosensitive poly(allylamine alpha-cyanocinnamylideneacetate). J Microencapsul 17, 245-521.

For example, microcapsules are prepared using modified collagen in a complex with a ter-polymer shell of 2-hydroxyethyl methylacrylate (HEMA), methacrylic acid (MAA), and methyl methacrylate (MMA), resulting in a capsule thickness of 2-5 μm. Such microcapsules can be further encapsulated with an additional 2-5 μm of ter-polymer shells in order to impart a negatively charged smooth surface and to minimize plasma protein absorption (Chia, S. M. et al. (2002). Multi-layered microcapsules for cell encapsulation. Biomaterials 23, 849-856).

Other microcapsules are based on alginate, a marine polysaccharide (Sambanis, A. (2003). Encapsulated islets in diabetes treatment. Diabetes Thechnol Ther 5, 665-668), or its derivatives. For example, microcapsules can be prepared by the polyelectrolyte complexation between the polyanions sodium alginate and sodium cellulose sulphate and the polycation poly(methylene-co-guanidine) hydrochloride in the presence of calcium chloride.

It will be appreciated that cell encapsulation is improved when smaller capsules are used. Thus, for instance, the quality control, mechanical stability, diffusion properties, and in vitro activities of encapsulated cells improved when the capsule size was reduced from 1 mm to 400 μm (Canaple, L. et al. (2002). Improving cell encapsulation through size control. J Biomater Sci Polym Ed 13, 783-96). Moreover, nanoporous biocapsules with well-controlled pore size as small as 7 nm, tailored surface chemistries, and precise microarchitectures were found to successfully immunoisolate microenvironments for cells (See: Williams, D. (1999). Small is beautiful: microparticle and nanoparticle technology in medical devices. Med Device Technol 10, 6-9; and Desai, T. A. (2002). Microfabrication technology for pancreatic cell encapsulation. Expert Opin Biol Ther 2, 633-646).

Examples of immunosuppressive agents which may be used in conjunction with the ex vivo treatment include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE®), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

For in vivo therapy, the composition (e.g., synthetic miR-135 molecule or conjugated form thereof) is administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the molecule accountable for the biological effect (e.g. synthetic miR-135 molecule or conjugated form thereof).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intranasal, ocular, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient, e.g. local injection into a diseased tissue.

According to a specific embodiment, the composition is for oral administration.

Conventional approaches for drug delivery to the central nervous system (CNS) include: neurosurgical strategies (e.g., intracerebral injection or intracerebroventricular infusion); molecular manipulation of the agent (e.g., production of a synthetic miR-135 molecule that is attached to a cell-targeting moiety that has an affinity for a brain cell surface molecule, as discussed above) in an attempt to exploit one of the endogenous transport pathways of the BBB; pharmacological strategies designed to increase the lipid solubility of an agent (e.g., conjugation of synthetic miR-135 molecule to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Methods for drug delivery behind the BBB include intracerebral implantation (such as with needles) and convection-enhanced distribution. Mannitol can be used in bypassing the BBB. Likewise, mucosal (e.g., nasal) administration can be used to bypass the BBB.

According to a specific embodiment, the composition is for intranasal administration.

Intranasal administration may be used for delivery of therapeutic agents to the central nervous system (CNS). The delivery occurs through the olfactory epithelium which is situated at the upper posterior part of the nasal cavity. The neurons of the olfactory epithelium project into the olfactory bulb in the brain hence enable a direct connection between the brain and the external environment. The transfer of drugs into the brain is thought to occur by either slow inner olfactory nerve cells transport or by a faster transfer along the perineural space surrounding the olfactory nerve cells into the cerebrospinal fluid in the brain. It is considered a non-invasive administration and allows large molecules that do not cross the BBB access to the CNS. This route of administration reduces systemic exposure and thus unwanted systemic side effects. Delivery from the nose to the CNS typically occurs within minutes and does not require the drug to bind to any receptor or axonal transport.

According to a specific embodiment, the composition is for intrathecal (IC), intracerebroventricular (ICV), ocular or intravenous (IV) administration, where the composition will allow passage through the blood brain barrier (BBB).

According to one embodiment, the pharmaceutical composition is administered via intrathecal administration i.e. into the spinal canal, or into the subarachnoid space so that it reaches the cerebrospinal fluid (CSF).

According to one embodiment, the pharmaceutical composition is administered via an ocular mode of administration.

According to one embodiment, the pharmaceutical composition is administered via an intracerebroventricular (ICV) mode of administration, i.e. by injection directly into the cerebrospinal fluid in cerebral ventricles.

According to one embodiment, the pharmaceutical composition is administered via an intravenous (IV) mode of administration.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. synthetic miR-135 molecule or conjugated form thereof) effective to prevent, alleviate or ameliorate symptoms of a disorder (e.g., CNS-related condition, e.g. psychiatric disorder such as a mood disorder) or prolong the survival of the subject being treated.

According to an embodiment of the present invention, administration of the synthetic miR-135 molecule or conjugated form thereof has an anti-depressant and anti-stress effect.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide sufficient plasma levels of the active ingredient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. The dosage and timing of administration will be responsive to a careful and continuous monitoring of the individual changing condition.

It will be appreciated that animal models exist by which the synthetic miR-135 molecules of the present invention may be tested prior to human treatment. For example, animal models of depression, stress, anxiety such as learned helplessness model (LH), chronic mild stress (CMS) model, social defeat stress (SDS) model and maternal deprivation model and sleep deprivation model may be used. For example, animal models of bipolar disease include, for example, transgenic mice with neuron-specific expression of mutant Polg (D181A) [as taught by Kato et al., Neuroscience and Biobehavioral Reviews (2007) 6 (31):832-842, incorporated herein by reference], as well as the well-established mania rat models of Amphetamine-induced hyperactivity [taught e.g. in U.S. Pat. No. 6,555,585] and Ketamine-induced hyperactivity [taught e.g. in Ghedim et al., Journal of Psychiatric Research (2012) 46: 1569-1575], incorporated by reference, may be used.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that the therapeutic compositions of the invention may comprise, in addition to the synthetic miR-135 molecule or conjugated form thereof, other known medications for the treatment of CNS-related conditions (e.g. psychiatric disorders, e.g. depression, stress, anxiety, sleep deprivation, etc.) such as, but not limited to, selective serotonin reuptake inhibitors (SSRIs), serotonin-norepinephrine reuptake inhibitors (SNRIs), noradrenergic and specific serotonergic antidepressants (NaSSAs), norepinephrine (noradrenaline) reuptake inhibitors (NRIs), norepinephrine-dopamine reuptake inhibitors, selective serotonin reuptake enhancers, norepinephrine-dopamine disinhibitors, tricyclic antidepressants (e.g. Imipramine), monoamine oxidase inhibitors (MAOIs). These medications may be included in the article of manufacture in a single or in separate packagings.

According to one embodiment, the therapeutic composition of the invention comprises, in addition to the synthetic miR-135 molecule or conjugated form thereof, a medicament or any combination of medicaments, including but not limited to, lithium (e.g. Lithium carbonate, Lithium citrate, Lithium sulfate), antipsychotic medicaments (e.g. typical antipsychotics and atypical antipsychotics, as detailed below), mood stabilizer medicaments (e.g. Valproic acid (VPA, Valproate), minerals, anticonvulsants, antipsychotics) and anti-depressants.

Exemplary typical antipsychotic medicaments which may be used in accordance with the present teachings, include but are not limited to, Low potency medicaments: Chlorpromazine (Largactil®, Thorazine®), Chlorprothixene (Truxal®), Thioridazine (Mellaril®), Mesoridazine and Levomepromazine; Medium potency medicaments: Loxapine (Loxapac®, Loxitane®), Molindone (Moban®), Perphenazine (Trilafon®) and Thiothixene (Navane®); High potency medicaments: Haloperidol (Haldol®, Serenace®), Fluphenazine (Prolixin®), Droperidol, Zuclopenthixol (Clopixol®), Flupentixol (Depixol®), Prochlorperazine and Trifluoperazine (Stelazine®). In addition, Prochlorperazine (Compazine®, Buccastem®, Stemetil®) and Pimozide (Orap®) may be used.

Exemplary atypical antipsychotic medicaments (also referred to as second generation antipsychotics) which may be used in accordance with the present teachings, include but are not limited to, Amisulpride (Solian®), Aripiprazole (Abilify®), Asenapine (Saphris®), Blonanserin (Lonasen®), Bitopertin (RG1678), Brexpiprazole (OPC-34712), Carpipramine (Prazinil®), Clocapramine (Clofekton®), Clozapine (Clozaril®), Cariprazine (RGH-188), Iloperidone (Fanapt®), Lurasidone (Latuda®), LY2140023, Melperone (Buronil®), Mosapramine (Cremin®), Olanzapine (Zyprexa®), Paliperidone (Invega®), Perospirone (Lullan®), Pimavanserin (ACP-103®), Quetiapine (Seroquel®), Remoxipride (Roxiam®), Risperidone (Risperdal®), Sertindole (Serdolect®), Sulpiride (Sulpirid®), Vabicaserin (SCA-136®), Ziprasidone (Geodon®), Zotepine (Nipolept®) and Zicronapine (Lu 31-130).

Exemplary mood stabilizers which may be used in accordance with the present teachings, include but are not limited to, minerals (e.g. lithium); anticonvulsant mood stabilizers including Valproic acid (Depakine®), divalproex sodium (Depakote®), and sodium valproate (Depacon®, Epilim®), Lamotrigine (Lamictal®), Carbamazepine (Tegretol®), Oxcarbazepine (Trileptal®), Topiramate (Topamax®), Riluzole (Rilutek®) and Gabapentin (Neurontin®); antipsychotics (as described above); and food supplements (e.g. omega-3 fatty acids).

Exemplary anti-depressants which may be used in accordance with the present teachings, include but are not limited to, Selective serotonin reuptake inhibitors (SSRIs, such as Citalopram, Escitalopram, Fluoxetine, Fluvoxamine, Paroxetine and Sertraline); Serotonin-norepinephrine reuptake inhibitors (SNRIs, such as Desvenlafaxine, Duloxetine, Milnacipran and Venlafaxine); Noradrenergic and specific serotonergic antidepressants (such as Mianserin and Mirtazapine); Norepinephrine (noradrenaline) reuptake inhibitors (NRIs, such as Atomoxetine, Mazindol, Reboxetine and Viloxazine); Norepinephrine-dopamine reuptake inhibitors (such as Bupropion); Selective serotonin reuptake enhancers (such as Tianeptine); Norepinephrine-dopamine disinhibitors (NDDIs such as Agomelatine); Tricyclic antidepressants (including Tertiary amine tricyclic antidepressants and Secondary amine tricyclic antidepressants); and Monoamine oxidase inhibitor (MAOIs).

According to one embodiment, the anti-depressant drug comprises selective serotonin reuptake inhibitors (SSRI), tricyclic antidepressants and noradrenaline reuptake inhibitors (NRI).

According to a specific embodiment, the anti-depressant drug comprises selective serotonin reuptake inhibitors (SSRI).

It will be appreciated that additional non-pharmaceutical therapeutic strategies may be employed in combination with the present teachings, including but not limited to, clinical psychology, electroconvulsive therapy, involuntary commitment, light therapy, psychotherapy, transcranial magnetic stimulation and cognitive behavioral therapy.

According to another aspect of the present invention there is provided a method of treating a CNS-related condition in a subject in need thereof, the method comprising administering to the subject a therapeutically effective amount of the composition of matter or conjugate of some embodiments of the invention, thereby treating the CNS-related condition.

According to another aspect of the present invention there is provided a therapeutically effective amount of the composition of matter or conjugate of some embodiments of the invention for use in treating a CNS-related condition in a subject in need thereof.

The term “treating” refers to inhibiting or arresting the development of a disease, disorder or condition and/or causing the reduction, remission, or regression of a disease, disorder or condition or keeping a disease, disorder or medical condition from occurring (i.e. preventing) in a subject who may be at risk for the disease disorder or condition, but has not yet been diagnosed as having the disease disorder or condition. Those of skill in the art will understand that various methodologies and assays can be used to assess the development of a disease, disorder or condition, and similarly, various methodologies and assays may be used to assess the reduction, remission or regression of a disease, disorder or condition.

As used herein, the term “subject” or “subject in need thereof” includes mammals, such as human beings, male or female, at any age which suffers from the pathology or is at risk to develop the pathology.

As used herein the phrase “CNS-related condition” or “central nervous system-related condition” includes psychiatric disorders (e.g., panic syndrome or panic attack, anxiety disorders, e.g. general anxiety disorder, phobic syndromes of all types, e.g. social phobia, mania, manic depressive disease, e.g. bipolar disease, hypomania, depression of all forms and/or types, e.g. unipolar depression, stress disorders, PTSD, somatoform disorders, personality disorders, compulsive behavior, psychosis, and schizophrenia), addiction or substance-related disorders e.g. drug dependence [e.g., alcohol, psychostimulants (e.g., crack, cocaine, speed, and meth), opioids, and nicotine], stress, fatigue, epilepsy, headache, acute pain, chronic pain, neuropathies, cereborischemia, dementia (e.g. Alzheimer's type and multi-infarct dementia), memory loss, cognition impairment (e.g. impaired cognitive function), sleep disorder (e.g. insomnia, early-morning awakening, and/or oversleeping), eating disorder (e.g. bulimia, anorexia, body image distortion, binge-eating disorders), autism spectrum disorder, Tourette's disorder, childhood disorders, movement disorders, multiple sclerosis, growth disorder, reproduction disorder, adjustment disorder, delirium. Additional disorders are described in, e.g., the Diagnostic and Statistical Manual (DSM) of Mental Disorders, Fifth Edition (DSM-5). Typically, such disorders have a complex genetic, biochemical, and/or environmental component.

According to a specific embodiment, the CNS-related condition is a psychiatric disorder.

According to a specific embodiment, the psychiatric disorder is a mood disorder.

Non-limiting examples of mood disorders include, but are not limited to, depression (i.e., depressive disorders), bipolar disorders, substance-induced mood disorders, alcohol-induced mood disorders, benzodiazepine-induced mood disorders, mood disorders due to general medical conditions, as well as many others. See, e.g., DSM-5 (described above).

According to a specific embodiment, the psychiatric disorder is a depressive disorder.

Non-limiting examples of depressive disorders include, but are not limited to, major depression disorder (MDD), atypical depression, melancholic depression, psychotic major depression or psychotic depression, catatonic depression, postpartum depression, seasonal affective disorder (SAD), acute depression, chronic depression (dysthymia), double depression, depressive disorder not otherwise specified, depressive personality disorder (DPD), recurrent brief depression (RBD), minor depressive disorder (minor depression), premenstrual syndrome, premenstrual dysphoric disorder, depression caused by chronic medical conditions (e.g., cancer, chronic pain, chemotherapy, chronic stress), and combinations thereof. Various subtypes of depression are described in, e.g., DSM-5.

According to a specific embodiment, the psychiatric disorder is a bipolar disorder.

Non-limiting examples of bipolar disorders include, but are not limited to, mania, acute mania, severe mania, hypomania, depression, moderate depression, dysthymia, severe depression, episodes of mania and/or depression, psychosis/psychotic symptoms (e.g. hallucinations, delusions), mixed bipolar state, bipolar I disorder (mania with or without major depression), bipolar II disorder (hypomania with major depression), rapid-cycling bipolar disorder, Cyclothymia and/or Bipolar Disorder Not Otherwise Specified (BD-NOS). See, e.g., DSM-5 (described above). Bipolar disorder is also known as manic depression.

According to a specific embodiment, the psychiatric disorder is a schizophrenia.

According to one embodiment, schizophrenia refers to a psychiatric disorder involving a withdrawal from reality by an individual. Symptoms comprise for at least a part of a month two or more of the following symptoms: delusions, hallucinations, disorganized speech, grossly disorganized or catatonic behavior, or negative symptoms (i.e., affective flattening, alogia, or avolition). Schizophrenia encompasses disorders such as, e.g., schizoaffective disorders. Diagnosis of schizophrenia is described in, e.g., DSM-5. Types of schizophrenia include, but are not limited to, paranoid, disorganized, catatonic, undifferentiated, and residual. See, e.g., DSM-5 (described above).

According to a specific embodiment, the CNS-related condition is an autism spectrum disorder.

According to one embodiment, autism spectrum disorder refers to a spectrum of neurodevelopmental disorders characterized by impaired social interaction and communication accompanied by repetitive and stereotyped behavior. Autism includes a spectrum of impaired social interaction and communication; however, the disorder can be roughly categorized into “high functioning autism” or “low functioning autism,” depending on the extent of social interaction and communication impairment. Individuals diagnosed with “high functioning autism” have minimal but identifiable social interaction and communication impairments (e.g., Asperger's syndrome). Additional information on autism spectrum disorders can be found in, e.g., DSM-5; Sicile-Kira and Grandin, Autism Spectrum Disorders: The Complete Guide to Understanding Autism, Asperger's Syndrome, Pervasive Developmental Disorder, and Other ASDs, 2004, Perigee Trade; and Duncan et al., Autism Spectrum Disorders [Two Volumes]: A Handbook for Parents and Professionals, 2007, Praeger.

According to a specific embodiment, the CNS-related condition is a neuroimmune-based psychiatric disorder. Non-limiting examples of neuroimmune-based psychiatric disorders include, but are not limited to, mood disorders such as depression (e.g., major depressive disorder) and bipolar disorder, schizophrenia, autism spectrum disorder, Pediatric Acute-onset Neuropsychiatric Syndrome (PANS) and Pediatric autoimmune neuropsychiatric disorder (PANDAS).

According to another aspect of the present invention there is provided a method of treating a depression-related disorder in a subject in need thereof, the method comprising administering to the subject a therapeutically effective amount of the composition of matter or conjugate of some embodiments of the invention, thereby treating the depression-related disorder.

According to another aspect of the present invention there is provided a therapeutically effective amount of the composition of matter or conjugate of some embodiments of the invention for use in treating a depression-related disorder in a subject in need thereof.

According to one embodiment, the depression-related disorder is selected from the group consisting of a major depression, an obsessive-compulsive disorder (OCD), a pervasive developmental disorder (PDD), a post-traumatic stress disorder (PTSD), an anxiety disorder, a bipolar disorder, an eating disorder and a chronic pain.

According to one embodiment, treating a CNS-related condition (e.g. a psychiatric disorder) or a depression-related disorder may be further effected by administering to the subject an additional medicament (or any combination of medicaments) for the treatment of the CNS-related condition (e.g. the psychiatric disorder) or the depression-related disorder.

Exemplary medicaments for the treatment of a psychiatric disorder or depression-related disorder which may be used in accordance with the present teachings include, but are not limited to, selective serotonin reuptake inhibitors (SSRIs), serotonin-norepinephrine reuptake inhibitors (SNRIs), noradrenergic and specific serotonergic antidepressants (NaSSAs), norepinephrine (noradrenaline) reuptake inhibitors (NRIs), norepinephrine-dopamine reuptake inhibitors, selective serotonin reuptake enhancers, norepinephrine-dopamine disinhibitors, tricyclic antidepressants (e.g. Imipramine), monoamine oxidase inhibitors (MAOIs).

According to one embodiment, treating a psychiatric disorder or a depression-related disorder may be further effected by administering to the subject an additional medicament (or any combination of medicaments) for the treatment of the psychiatric disorder or depression-related disorder including but not limited to, lithium (e.g. Lithium carbonate, Lithium citrate, Lithium sulfate), antipsychotic medicaments (e.g. typical antipsychotics and atypical antipsychotics, as detailed hereinabove), mood stabilizer medicaments (e.g. Valproic acid (VPA, Valproate), minerals, anticonvulsants, antipsychotics) and anti-depressants. Additional medicaments which may be used in accordance with the present teachings are described in detail hereinabove.

According to one embodiment, an efficient treatment (e.g. psychiatric disorder treatment such as anti-depressant/mood disorder treatment) is determined when a significantly higher expression level of the miR-135 is obtained following to the treatment as compared to the miR-135 expression level prior to the treatment.

The expression level of miR-135 in a subject following treatment may be higher by about 5%, 10%, 15%, 20%, 25%, 30%, 40%, 50%, 60%, 70%, 80%, 90% or 100% as compared to that of the subject prior to treatment.

Monitoring treatment may also be effected by assessing the patient's well-being, and additionally or alternatively, by subjecting the subject to behavioral tests, MRI or any other method known to one of skill in the art.

According to another aspect of the present invention there is provided a method of treating a cancerous disease in a subject in need thereof, the method comprising administering to the subject a therapeutically effective amount of the composition of matter or conjugate of some embodiments of the invention, thereby treating the cancerous disease.

According to another aspect of the present invention there is provided a therapeutically effective amount of the composition of matter or conjugate of some embodiments of the invention for use in treating a cancerous disease in a subject in need thereof.

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a specific embodiment, the cancer comprises ovarian cancer, colorectal cancer, or prostate cancer.

According to one embodiment, treating a cancerous disease may be further effected by administering to the subject an additional medicament (or any combination of medicaments) for the treatment of the cancerous disease.

Such medicaments may include, without being limited to, radiation therapy, chemotherapy, biological therapy, e.g., immunotherapy or bone marrow transplantation.

According to one embodiment, an efficient anti-cancer treatment is determined when there is a decrease of at least about 10%, 20%, 30%, 40%, 50%, 60%, 70%, 80%, 90% or 100% or more in tumor mass or there is a halt in tumor growth, as compared to a subject not treated by the composition of the invention, or compared to the same subject being treated but prior to the treatment.

Those of skill in the art will understand that various methodologies and assays can be used to assess the efficiency of cancer treatment, e.g. CT scan, MRI, X-ray, ultrasound, blood tests etc.

The composition of matter of some embodiments of the invention may further be used to enhance muscle cell differentiation and bone regeneration.

Accordingly, a therapeutically effective amount of the composition of matter or conjugate of some embodiments of the invention may be used for treating a bone-related disease or condition or a muscle-related disease or condition in a subject in need thereof.

According to one embodiment, there is provided a method of promoting bone regeneration or muscle cell differentiation in a subject in need thereof, the method comprising administering to the subject a therapeutically effective amount of the composition of matter or conjugate of some embodiments of the invention, thereby promoting bone regeneration or muscle cell differentiation.

Exemplary bone related diseases or conditions which may be treated according to the present teachings include, but not limited to, injuries involving bone damage; fractures such as closed, open and non-union fractures; growth deficiencies; osteolytic bone disease such as cancer; periodontal disease and defects, and other tooth repair processes; skeletal disorders, such as age-related osteoporosis, post-menopausal osteoporosis, glucocorticoid-induced osteoporosis or disuse osteoporosis and arthritis, osteoarthritis or any condition that benefits from stimulation of bone formation. The compositions of the present invention can also be useful in treatment of congenital, trauma-induced or surgical resection of bone (for instance, for cancer treatment), and in cosmetic surgery.

Exemplary muscle related diseases or conditions which may be treated according to the present teachings include, but not limited to, muscle degeneration diseases, neuromuscular diseases, spinal muscular atrophies, inflammatory muscle diseases and metabolic muscle diseases. Exemplary muscle disease include, but are not limited to, Muscular Dystrophies e.g. Duchenne muscular dystrophy (DMD), Becker muscular dystrophy, facioscapulohumeral muscular dystrophy, and myotonic dystrophy; Amyotrophic lateral sclerosis (ALS), Myasthenia Gravis, Lambert-Eaton syndrome, Botulism and cerebrovascular accident.

According to one embodiment, an efficient treatment (e.g. bone regeneration and muscle cell differentiation) is determined when there is an increase of at least about 10%, 20%, 30%, 40%, 50%, 60%, 70%, 80%, 90% or 100% or more in bone regeneration (e.g. in bone cell mass) or in muscle cell differentiation (e.g. in muscle cell mass), as compared to a subject not treated by the composition of the invention, or compared to the same subject being treated but prior to the treatment.

Those of skill in the art will understand that various methodologies and assays can be used to assess the promotion of bone regeneration and muscle cell differentiation, e.g. CT scan, MRI, X-ray, ultrasound, blood tests etc.

It is expected that during the life of a patent maturing from this application many relevant miRNA modifications will be developed and the scope of the term modification is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 10 is expressed in a RNA sequence format (e.g., reciting U for uracil), but it can refer to either a RNA sequence that corresponds to an miR-135b nucleic acid sequence, or the DNA sequence of an miR-135b molecule nucleic acid sequence. Furthermore, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Animals and Housing

Adult C57BL/6 male mice 9-11 weeks old were used. Mice were housed in a temperature-controlled room (22±1° C.) on a reverse 12 hour light/dark cycle. Food and water were available ad libitum. All experimental protocols were performed on male mice and were approved by the Institutional Animal Care and Use Committee of The Weizmann Institute of Science.

Microdissection and Preparation of RNA

Immediately after decapitation, the brain was removed and placed into a 1 mm metal matrix (cat #51380; Stoelting Co. Wood Dale, IL). The brain was sliced using standard razor blades (GEM, 62-0165) into 1 mm or 2 mm slices that were quickly frozen on dry ice. Blunted syringes of different diameters were used to extract the brain regions from slices removed from the matrix which were then stored at −80° C. RNA extraction was performed using NucleoSpin™ RNA XS kit (Macherey-Nagel™, Duren, Germany). RNA was reverse transcribed to cDNA using the High Capacity RNA to cDNA kit (Applied Biosystems, Foster City, CA). The cDNA was then analyzed by quantitative real-time PCR (RT-PCR).

microRNA Purification and Quantitative Real Time PCR Expression Analysis

mRNAs including microRNAs were isolated using miRNeasy® mini kit (Qiagen) according to the manufacturer instructions, and treated using miScript® Reverse transcription kit to generate cDNA. Samples were then analyzed using SYBR™ Green PCR kit (Qiagen) according to the manufacturer's guidelines in Applied Biosystems™ 7500 thermocycler (Applied Biosystems). Specific primers for each miR were used together with the commercial universal primer, while U6 snRNA was used as an internal control. For mRNA quantification, specific primers were designed for each transcript using the software Primer Express 2 (Applied Biosystems) and expression was tested using real time PCR.

Cloning of Target Transcripts 3′UTRs into psiCHEK-2 Luciferase Expression Plasmid

3′UTRs sequences of Slc6a4 and Htr1a were PCR amplified from mouse genomic DNA. 3′UTRs PCR fragments were ligated into pGEM®-T easy vector (Promega) according to the manufacturer's guidelines, and further sub-cloned into a single NotI site at the 3′ end of luciferase in the psiCHECK-2 reporter plasmid (Promega). Cloning orientation was verified by diagnostic cuts and sequencing.

Transfections and luciferase assays HEK293T cells were grown on poly-L-lysine in a 84-well plate to 70-85% confluence and transfected using polyethyleneimine with the following plasmids: psiCHECK-2 plasmid containing the wild type or mutated 3′UTR and the overexpressing vector for a specific miRNA, or empty overexpression plasmids. miRs overexpression plasmids were adopted from the miR-Vec library. Twenty-four hours following transfection, cells were lysed and luciferase reporter activities were assayed as previously described [Kuperman Y. et al., Mol Endocrinol (2011) 25: 157-169]. Renilla luciferase values were normalized to control firefly luciferase levels, transcribed from the same vector but not affected by 3′UTR, then tested and averaged across six repetitions per condition.

TABLE 2
Oligonucleotide primers used for cloning

| Gene | Orientation | Primer Sequence |
|---|---|---|
| Slc6a4 3′UTR | sense | ATCCGCATGAATGCTGTGTA |
| | | (SEQ ID NO: 17) |
| Slc6a4 3′UTR | antisense | GTGGGTGGTGGAAGAGACAC |
| | | (SEQ ID NO: 18) |
| Htr1a 3′UTR | sense | AGTTCTGCCGCTGATGATG |
| | | (SEQ ID NO: 19) |
| Htr1a 3′UTR | antisense | GCACAAATGGAGAGTCTGATTAAA |
| | | (SEQ ID NO: 20) |

TABLE 3
Oligonucleotide primers used for
microRNA real time PCR

| Gene | Primer sequence |
|---|---|
| miR-135a | TATGGCTTTTTATTCCTATGTGA |
| | (SEQ ID NO: 21) |
| miR-135b | TATGGCTTTTCATTCCTATGTGA |
| | (SEQ ID NO: 22) |
| miR-375 | TTTGTTCGTTCGGCTCGCGTGA |
| | (SEQ ID NO: 23) |
| U6 | GATGACACGCAAATTCGTGAA |
| | (SEQ ID NO: 24) |
| miR-124 | TAAGGCACGCGGTGAATGCC |
| | (SEQ ID NO: 25) |
| miR-16 | TAGCAGCACGTAAATATTGGCG |
| | (SEQ ID NO: 26) |

TABLE 4
Oligonucleotide primers used for
mRNA real time PCR

| Gene | Sense primer | Antisense primer |
|---|---|---|
| Slc6a4 | GGGTTTGGATAGTACGTT | CATACGCCCCTCCTGATGTC |
| | CGCA | (SEQ ID NO: 28) |
| | (SEQ ID NO: 27) | |
| Htr1a | GTGCACCATCAGCAAGGA | GCGCCGAAAGTGGAGTAGAT |
| | CC | (SEQ ID NO: 30) |
| | (SEQ ID NO: 29) | |
| Hprt1 | TGACACTGGCAAAACAAT | GGTCCTTTTCACCAGCAAGCT |
| | GCA | (SEQ ID NO: 32) |
| | (SEQ ID NO: 31) | |
| Gapd | TGCACCACCAACTGCTTA | GGCATGGACTGTGGTCATGAG |
| | GC | (SEQ ID NO: 34) |
| | (SEQ ID NO: 33) | |
| YWHAZ | ACTTTTGGTACATTGTGG | CCGCCAGGACAAACCAGTAT |
| | CTTCAA | (SEQ ID NO: 36) |
| | (SEQ ID NO: 35) | |

### Synthesis of miR-135 Single Strand Oligonucleotide (Optionally Conjugated to Sertraline)

Oligonucleotide sequences were assembled using a standard procedure using standard 2′-deoxy, 2′-O-Me or 2′-MOE phosphoramidite blocks, e.g.:

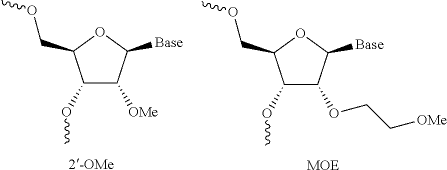

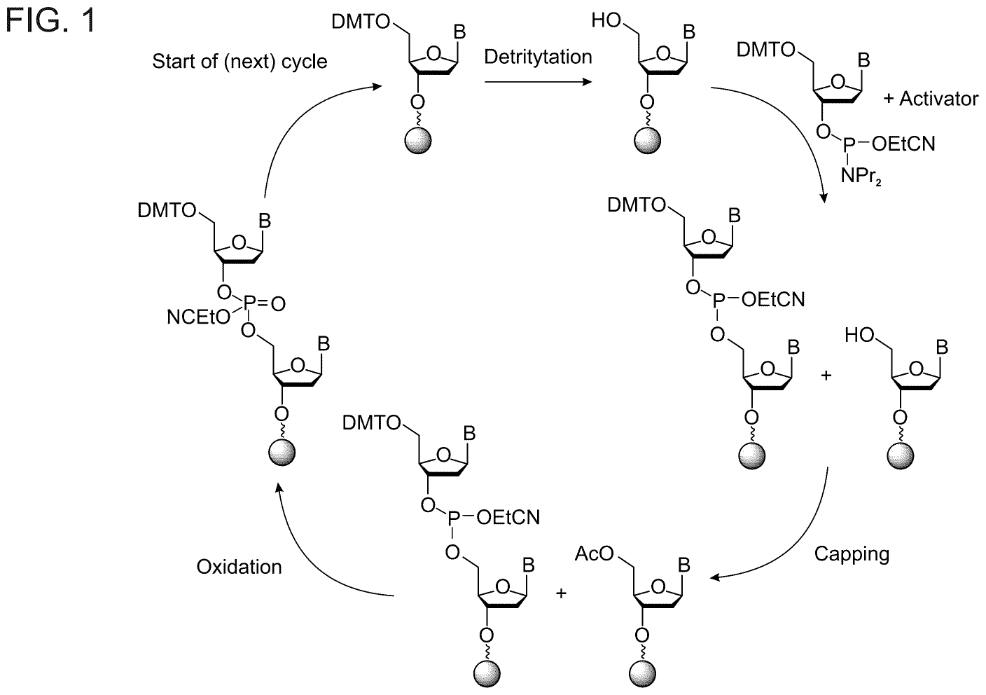

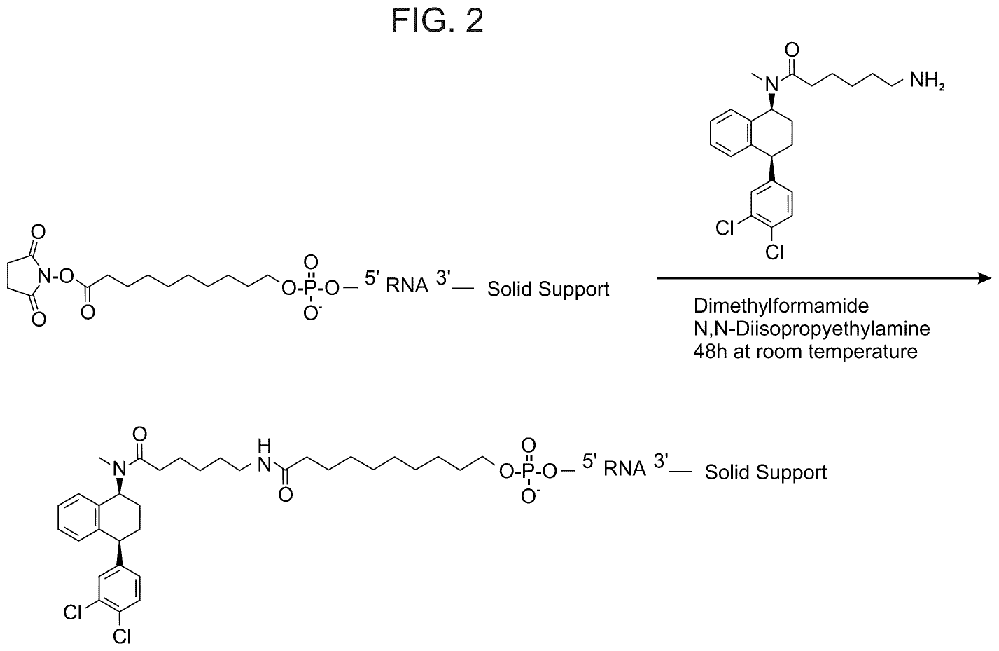

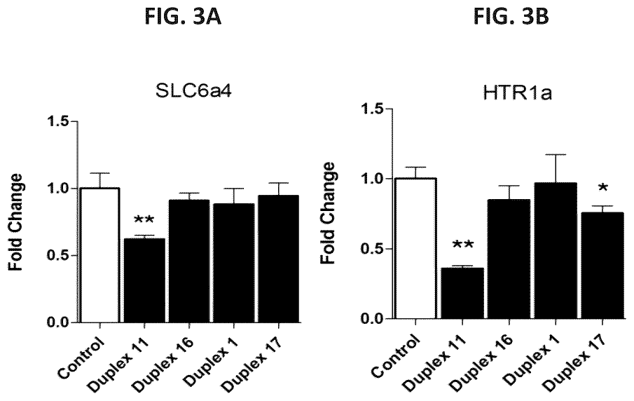

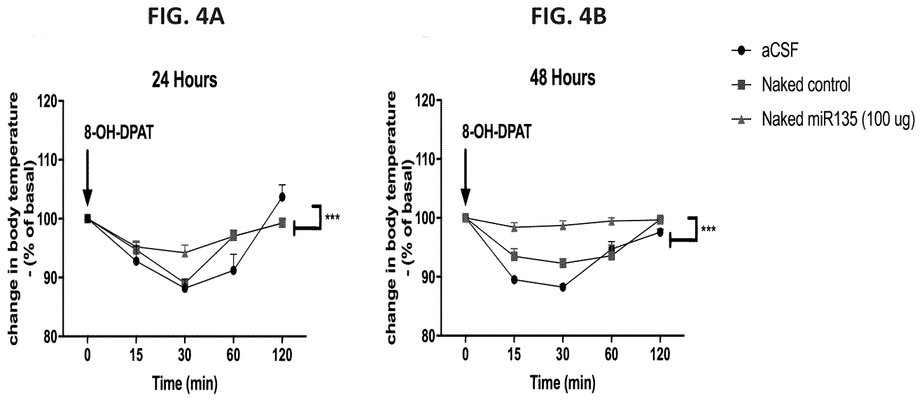

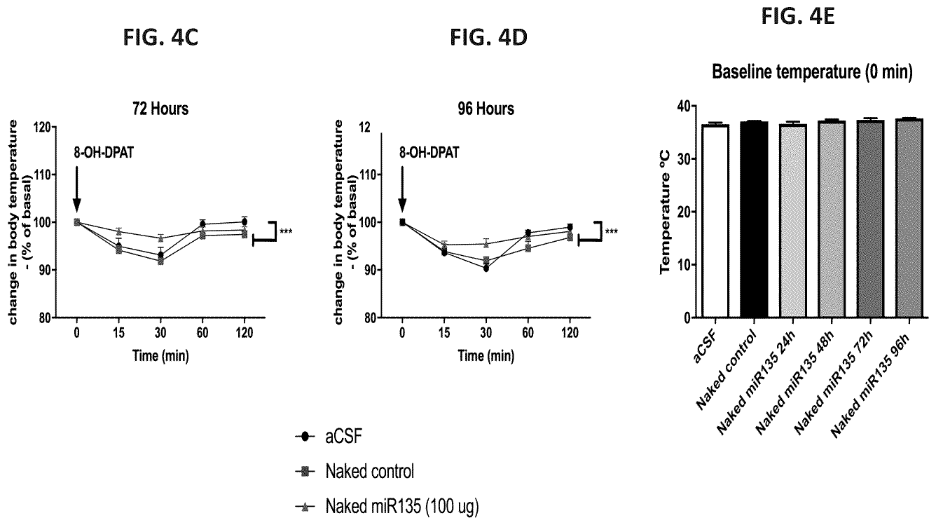

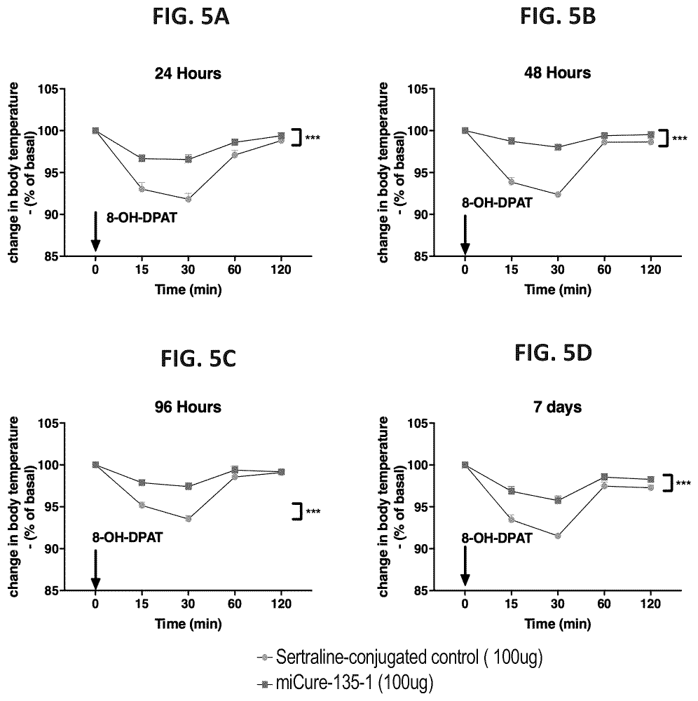

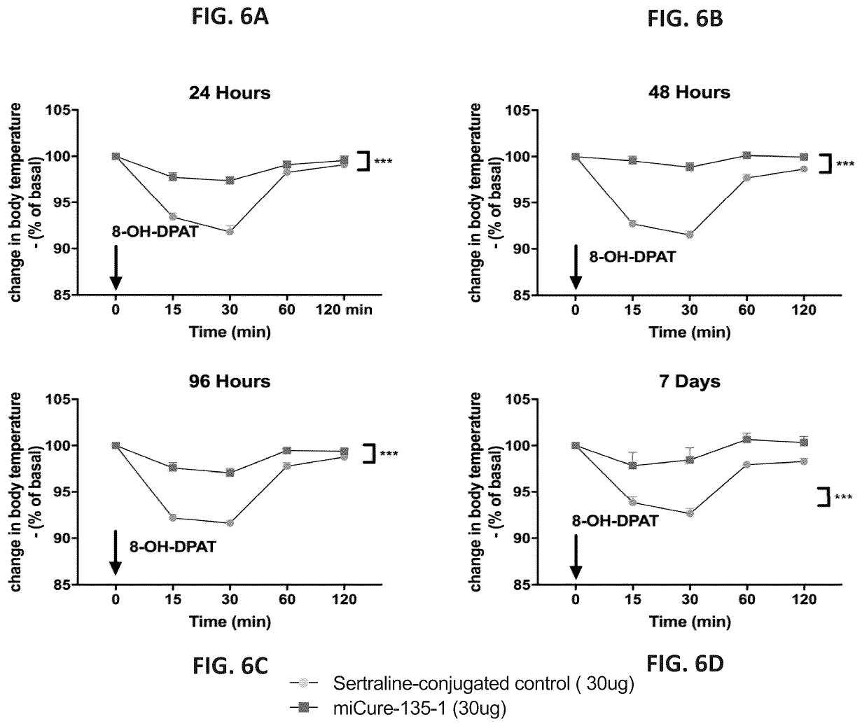

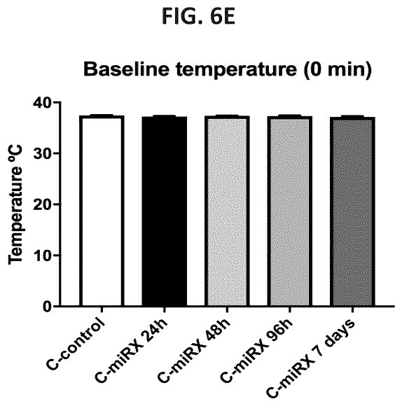

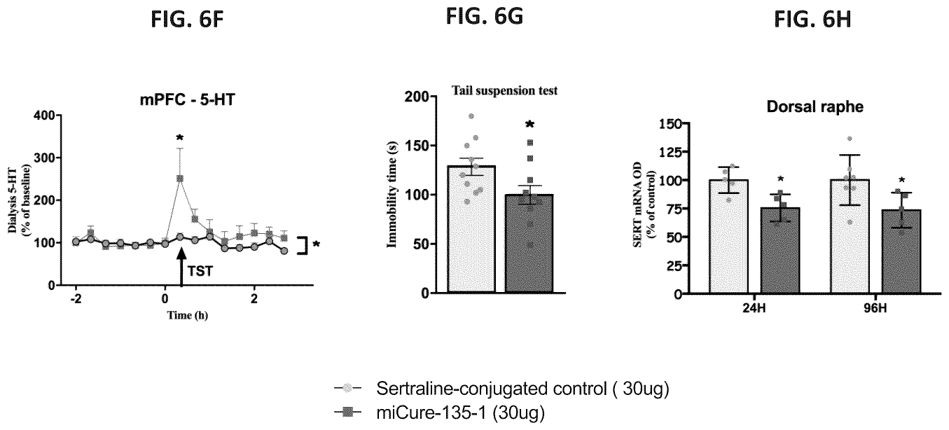

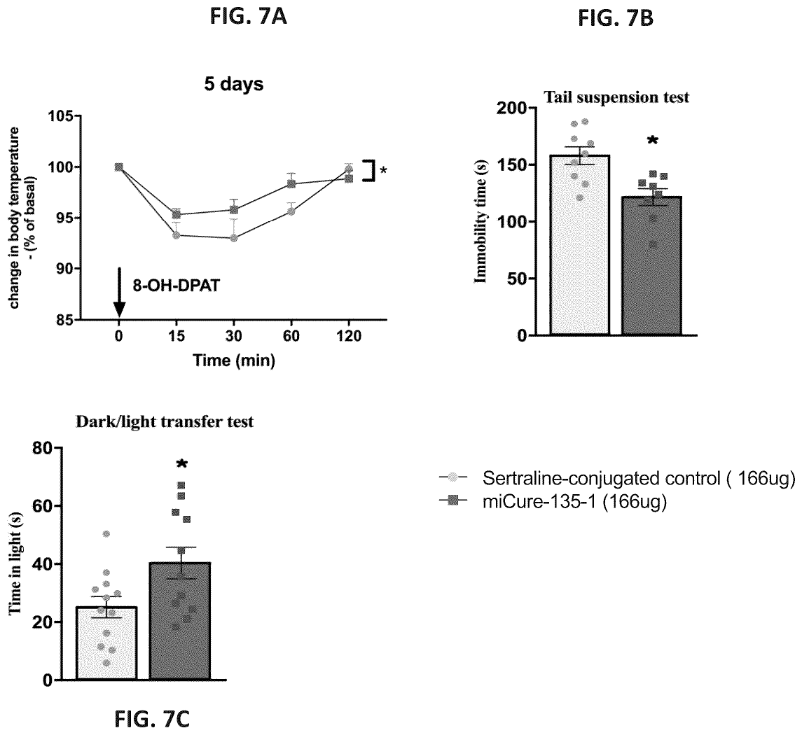

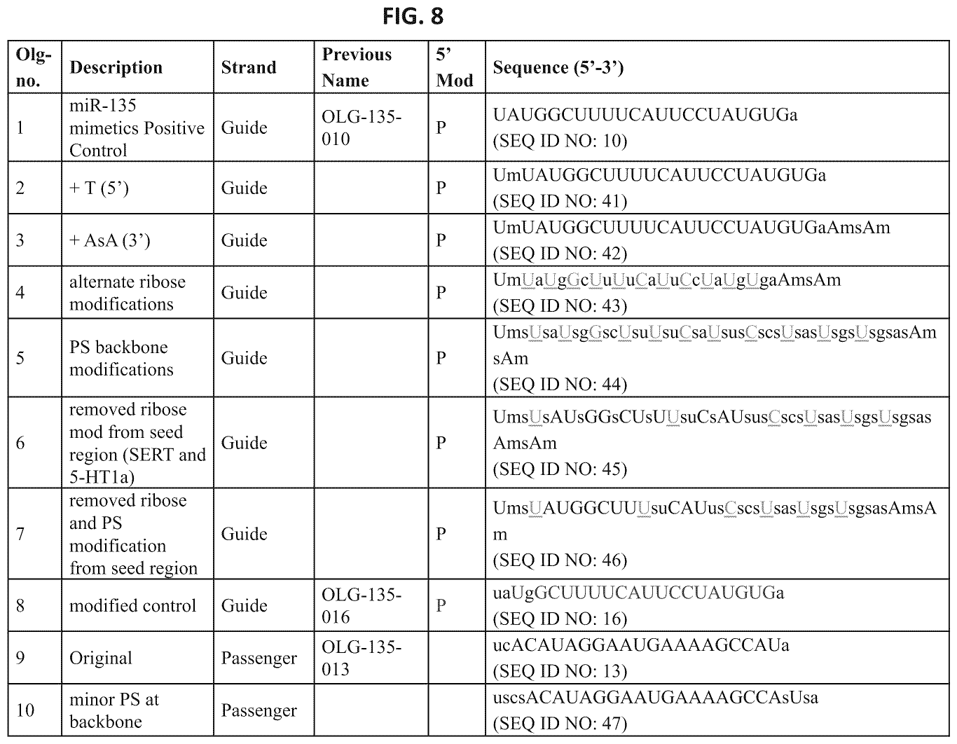

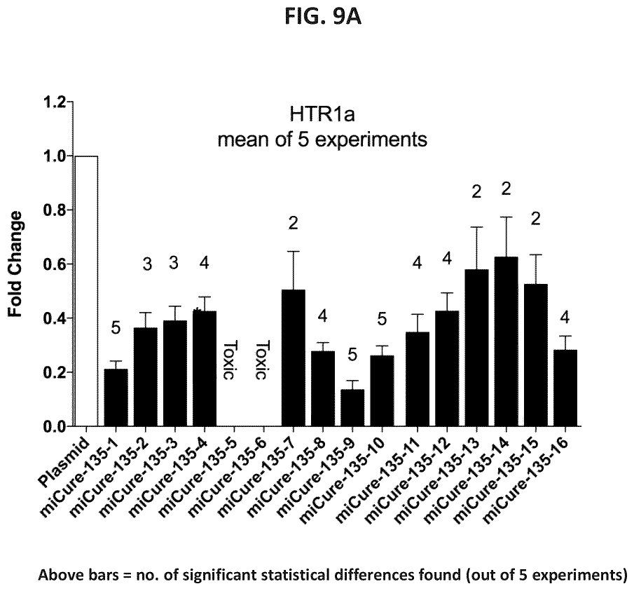

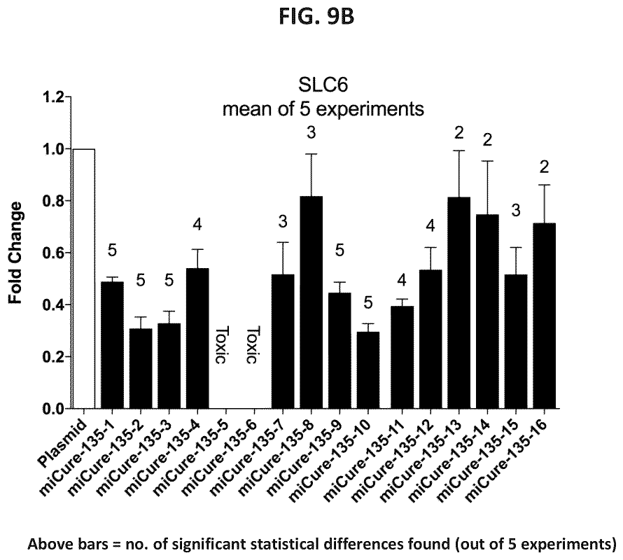

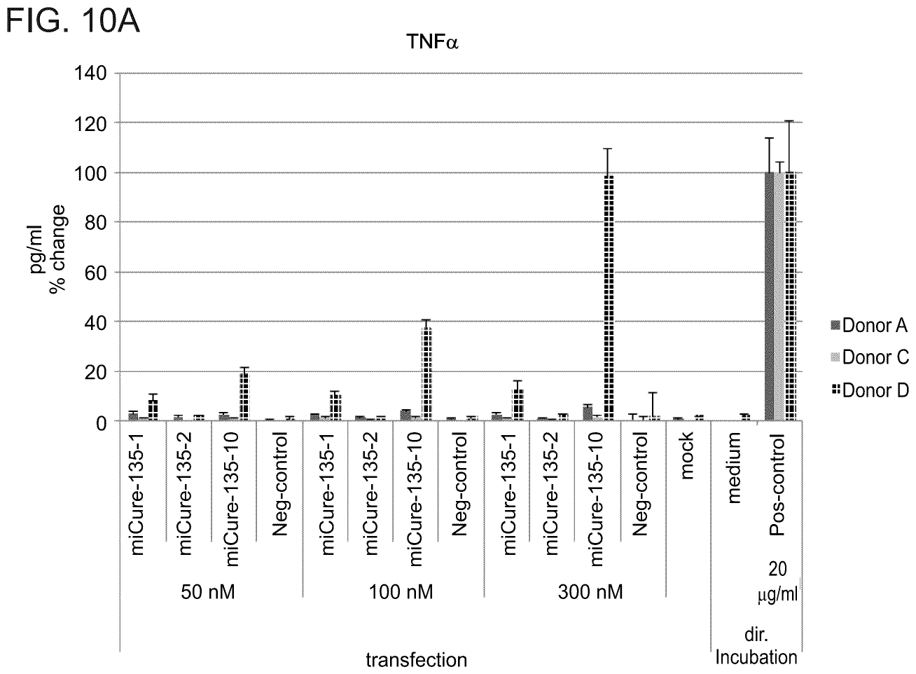

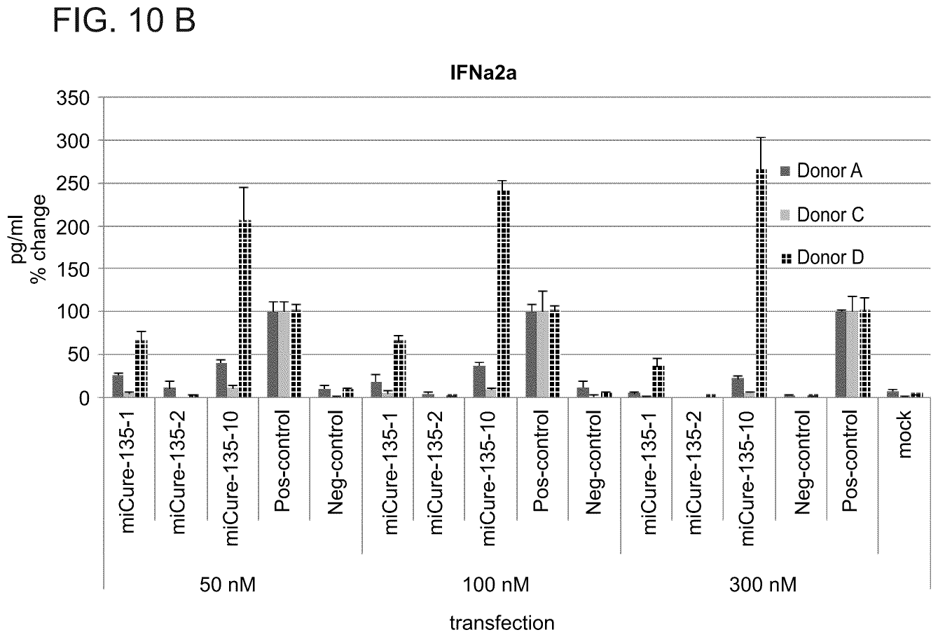

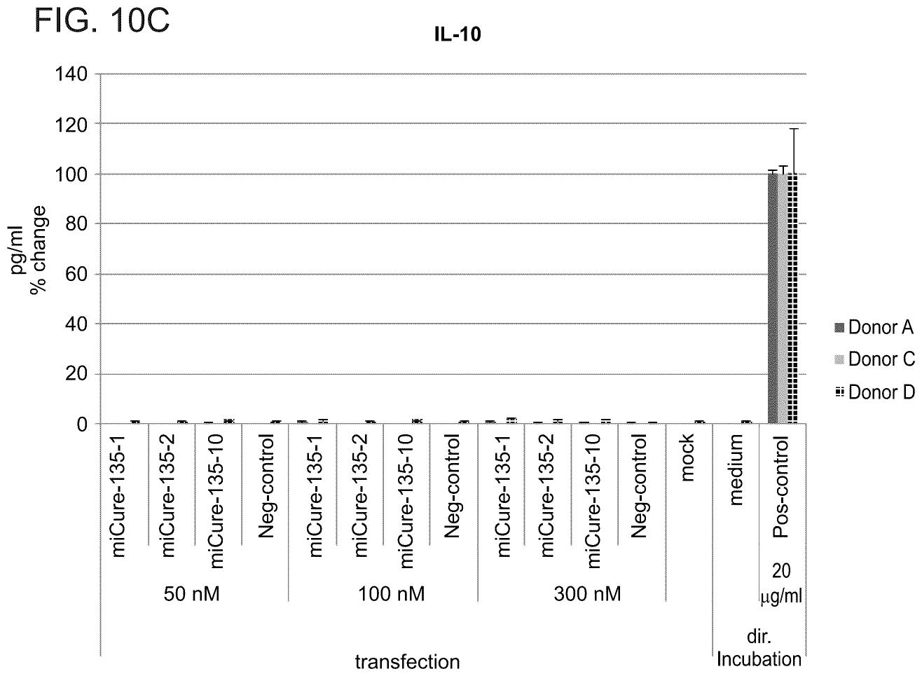

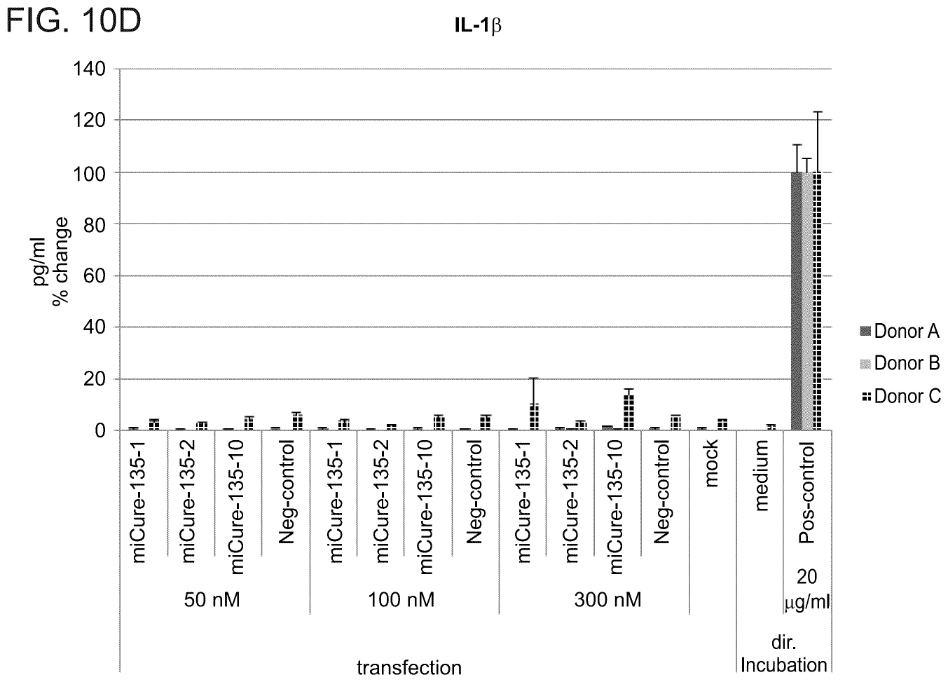

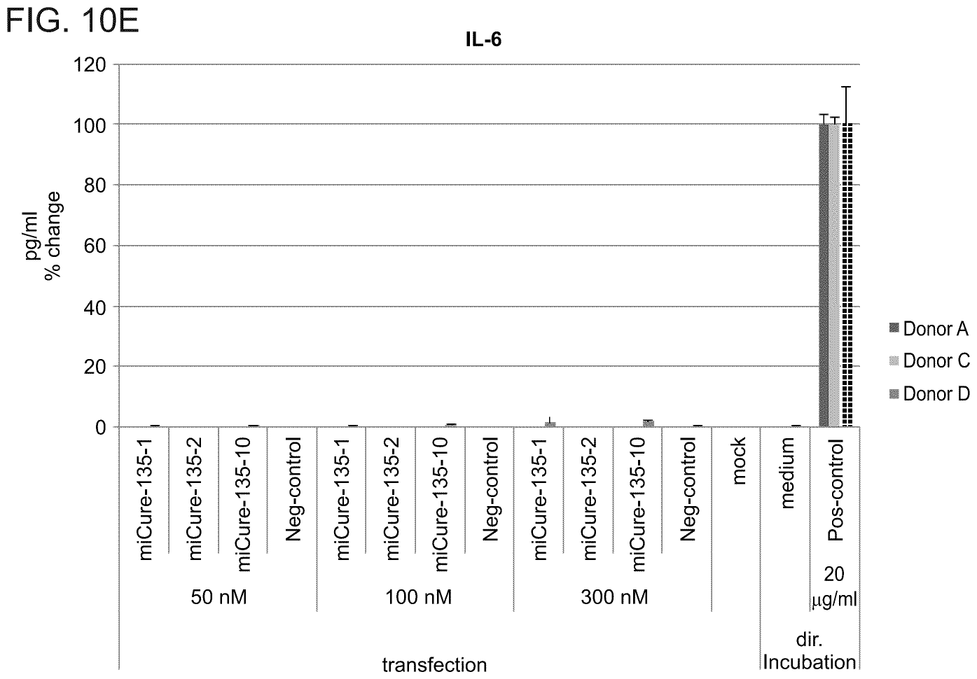

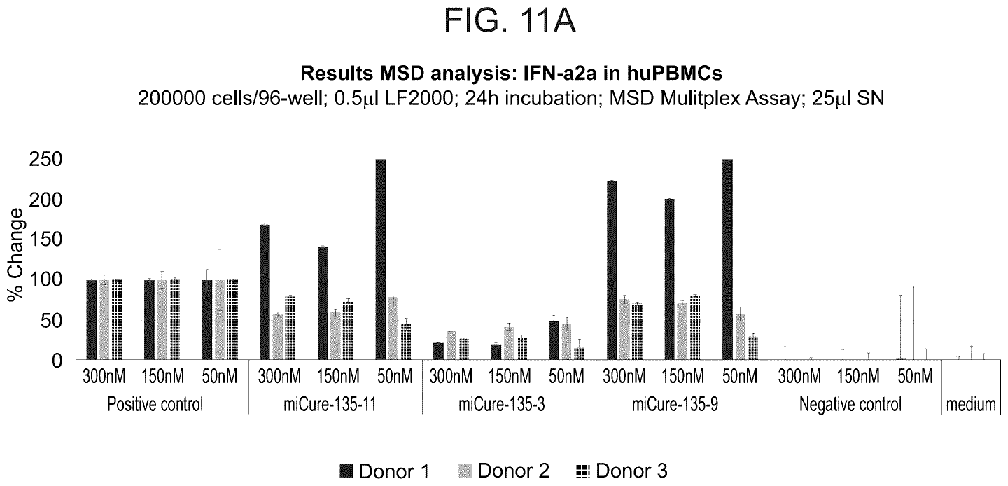

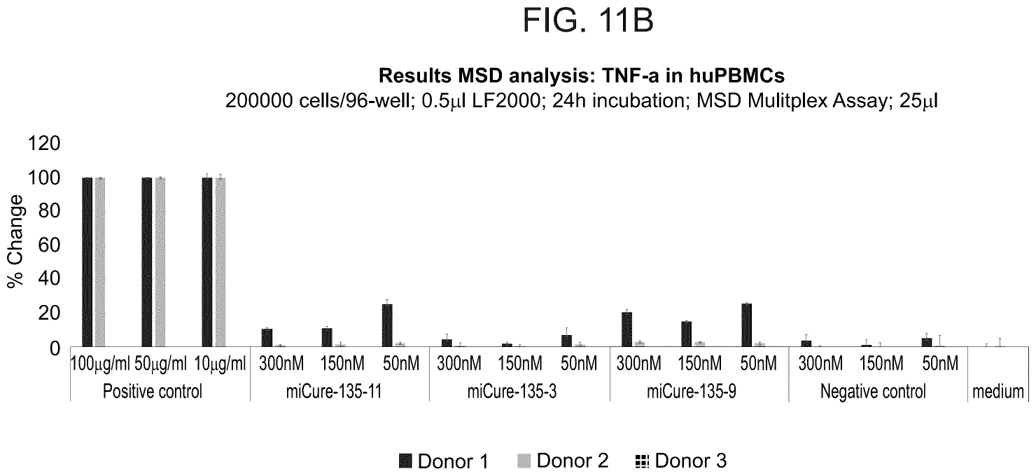

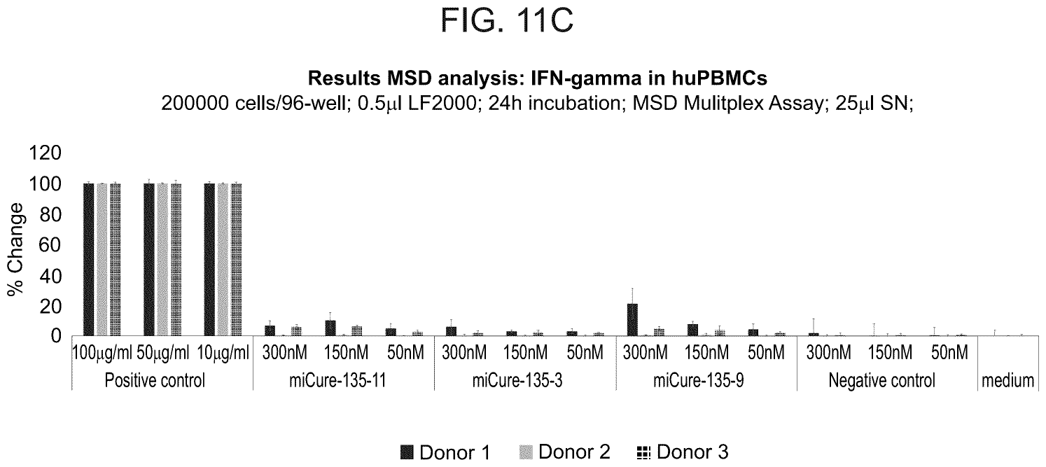

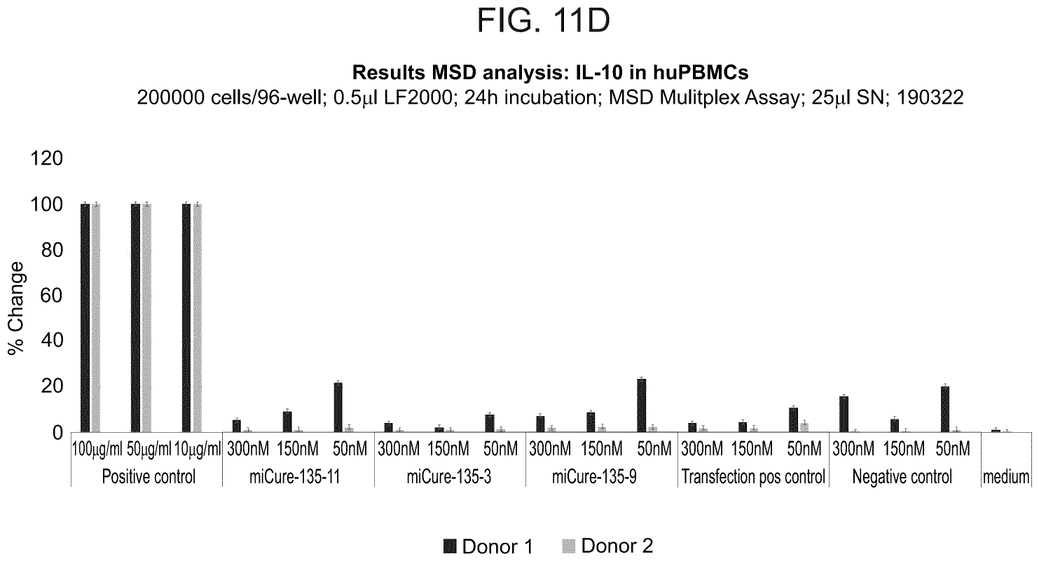

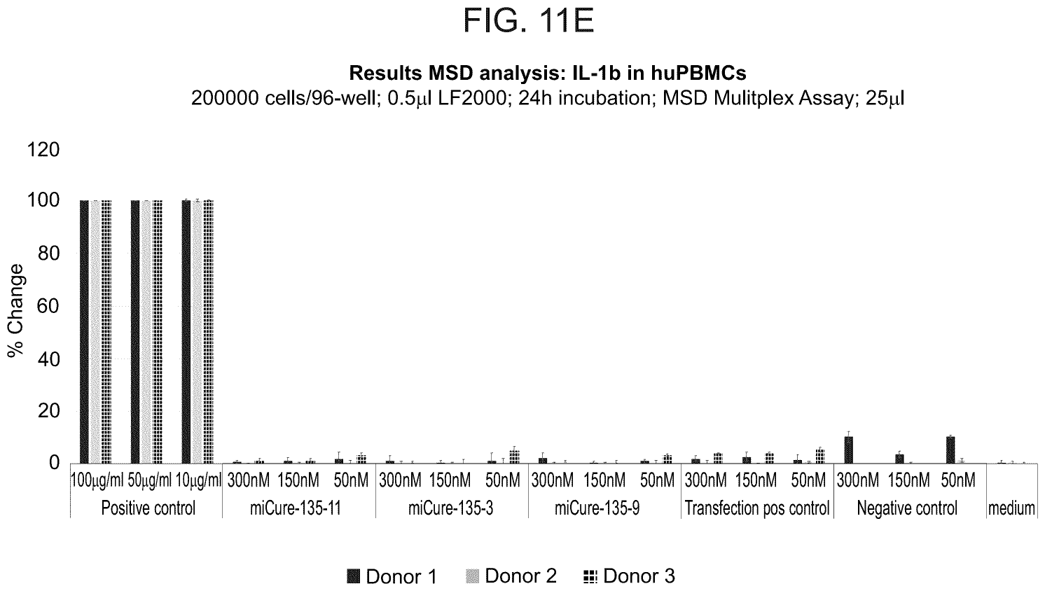

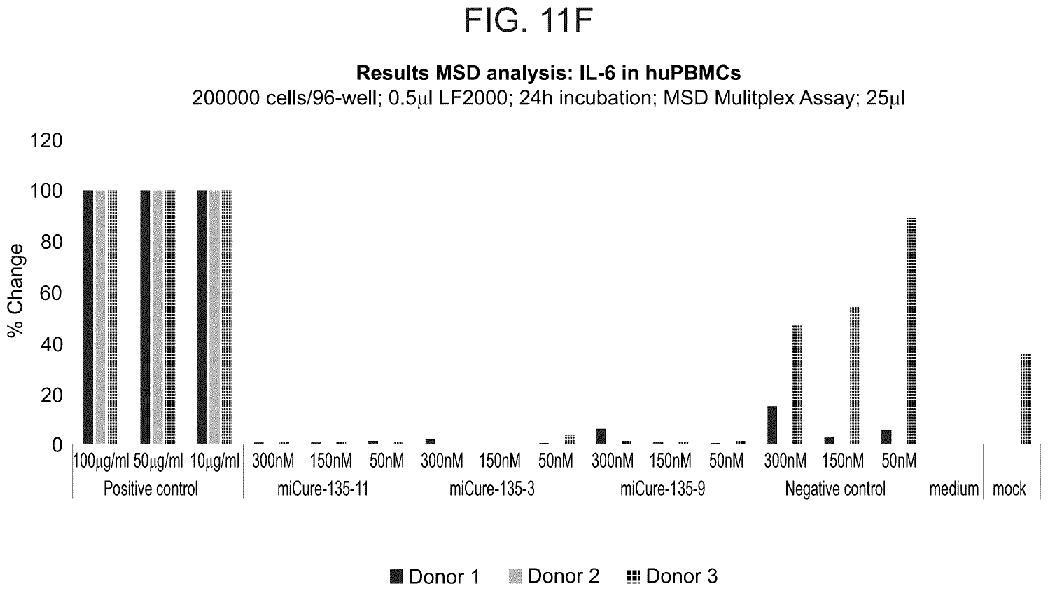

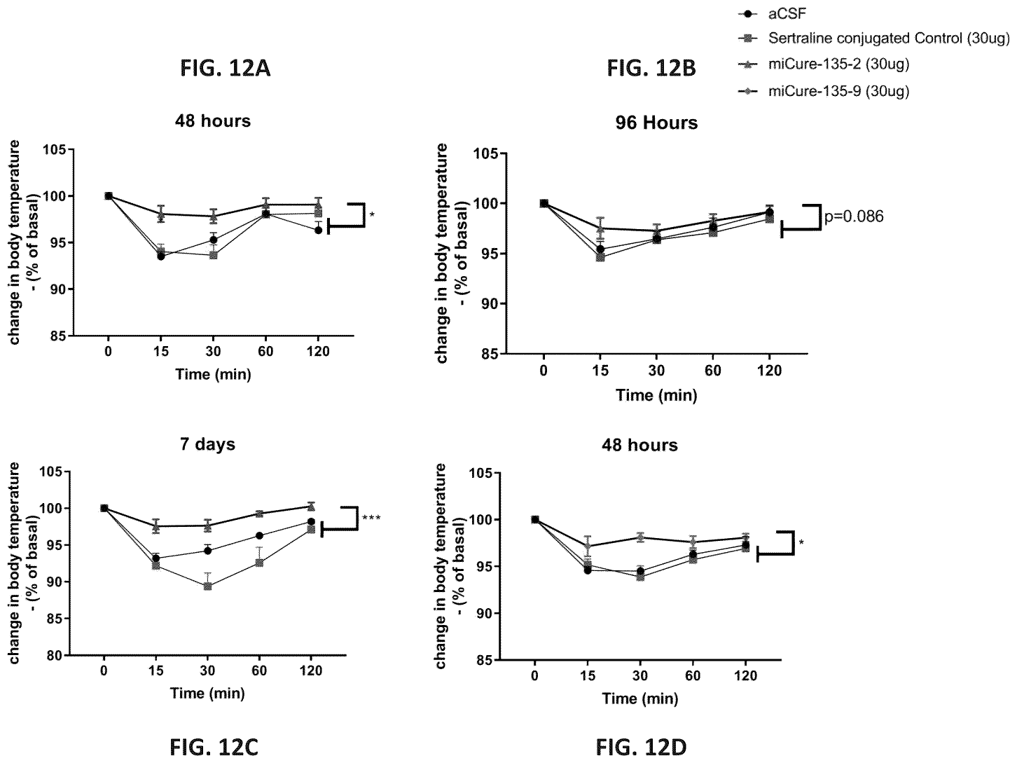

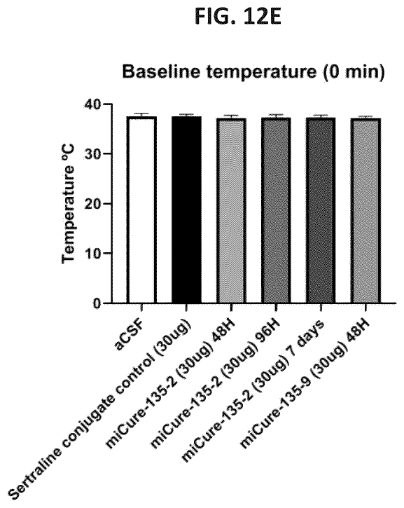

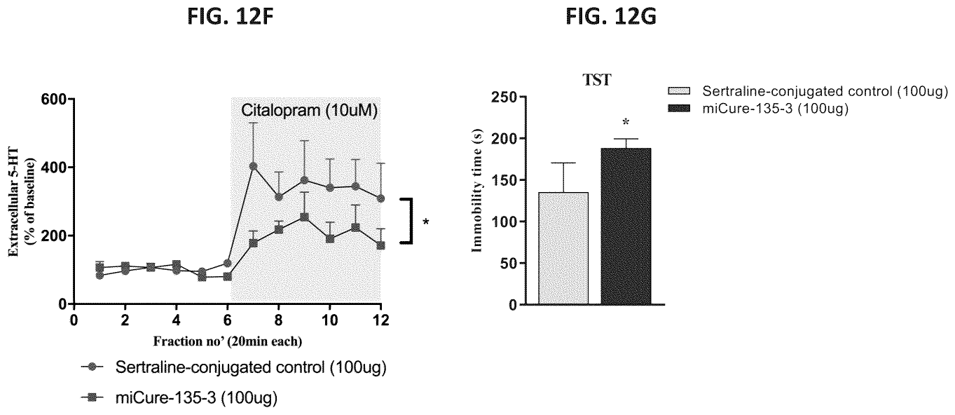

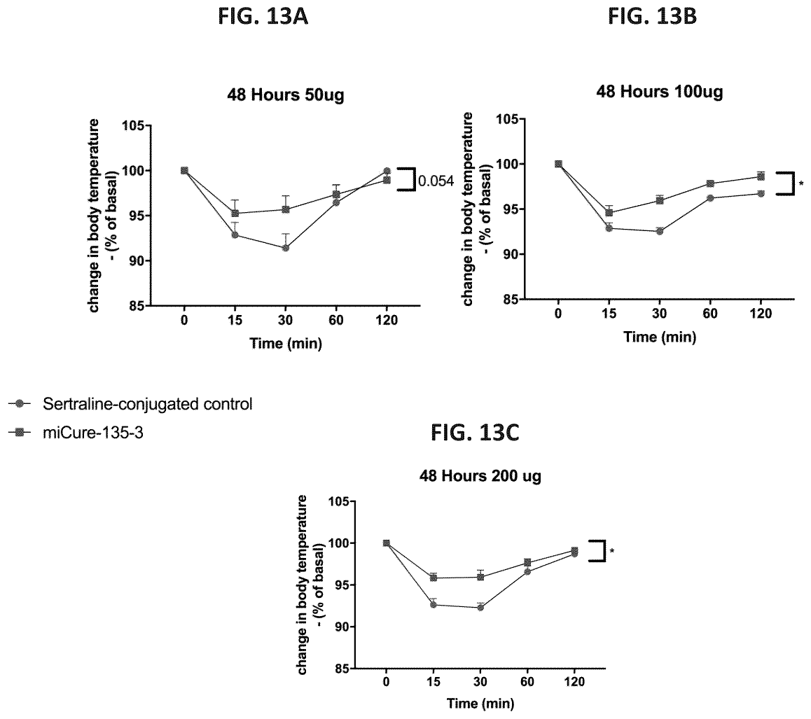

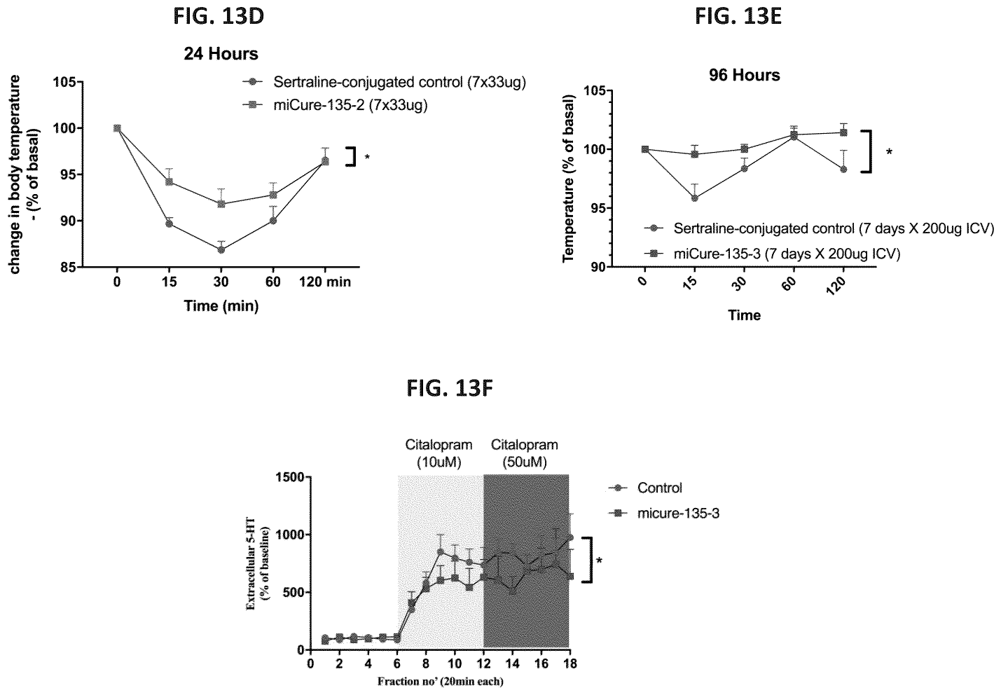

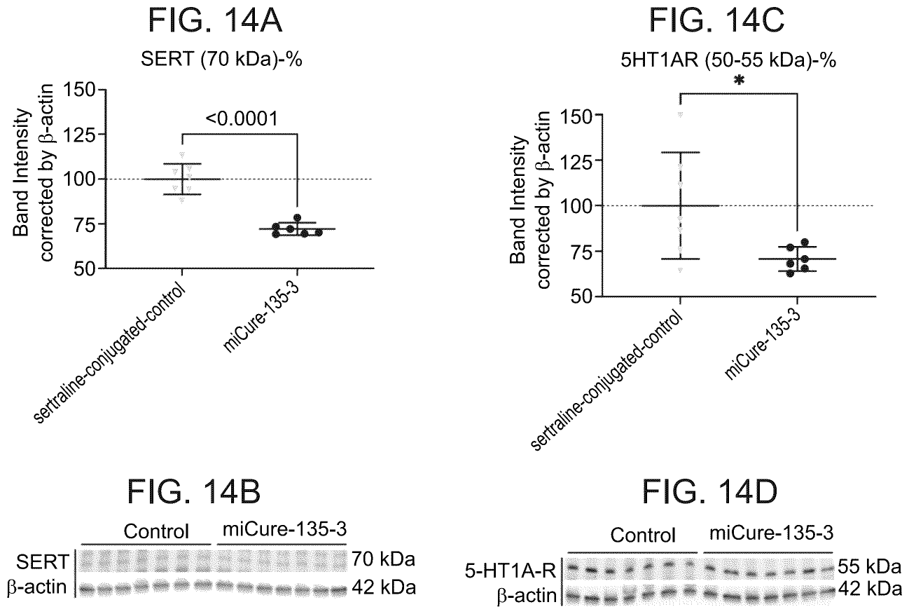

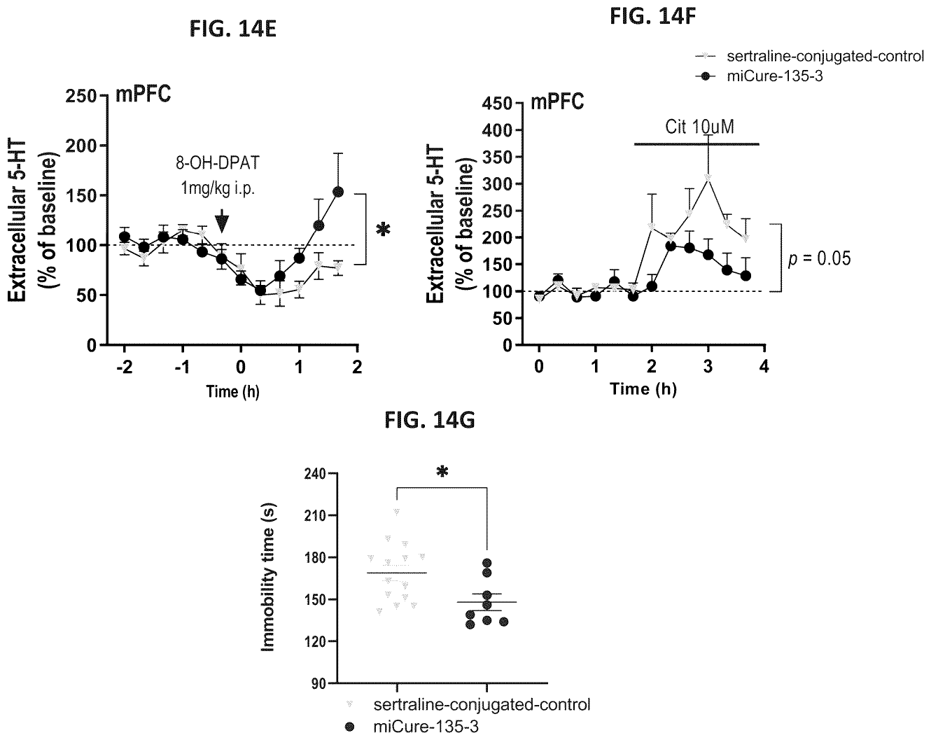

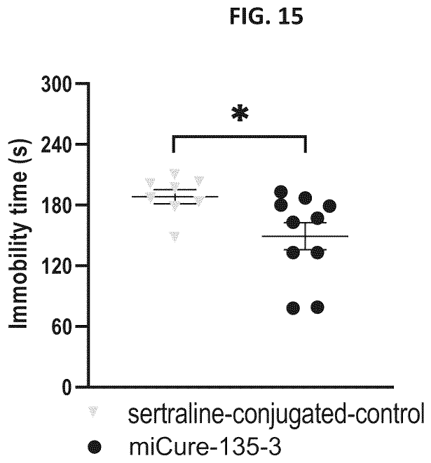

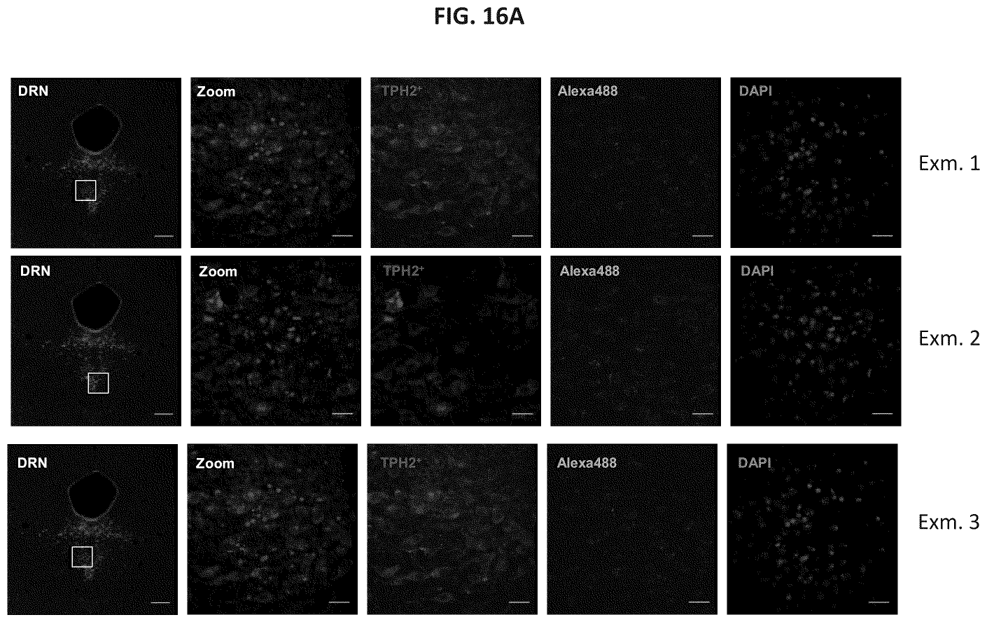

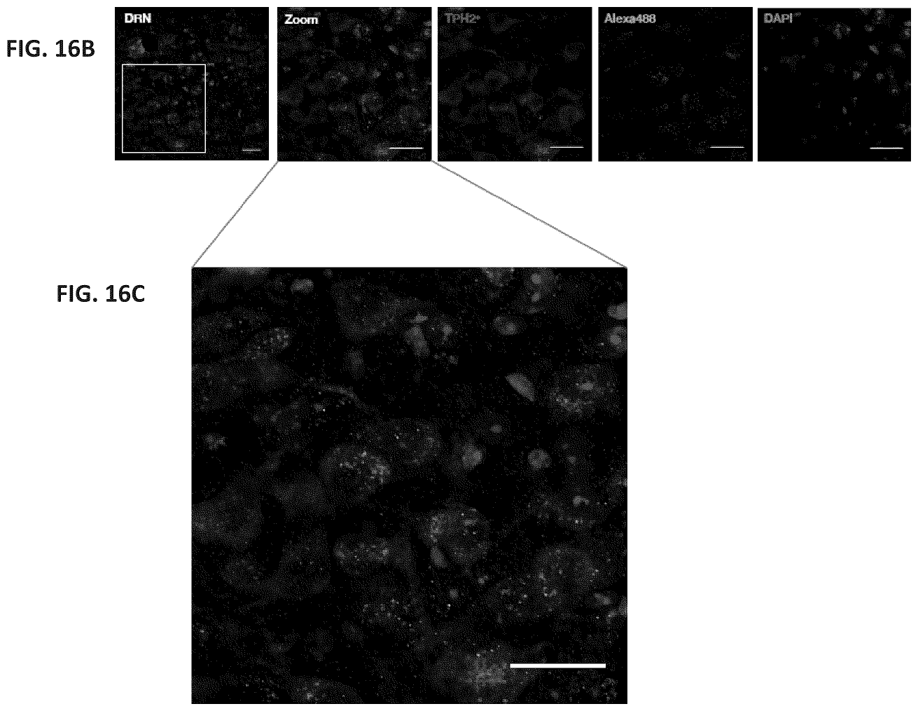

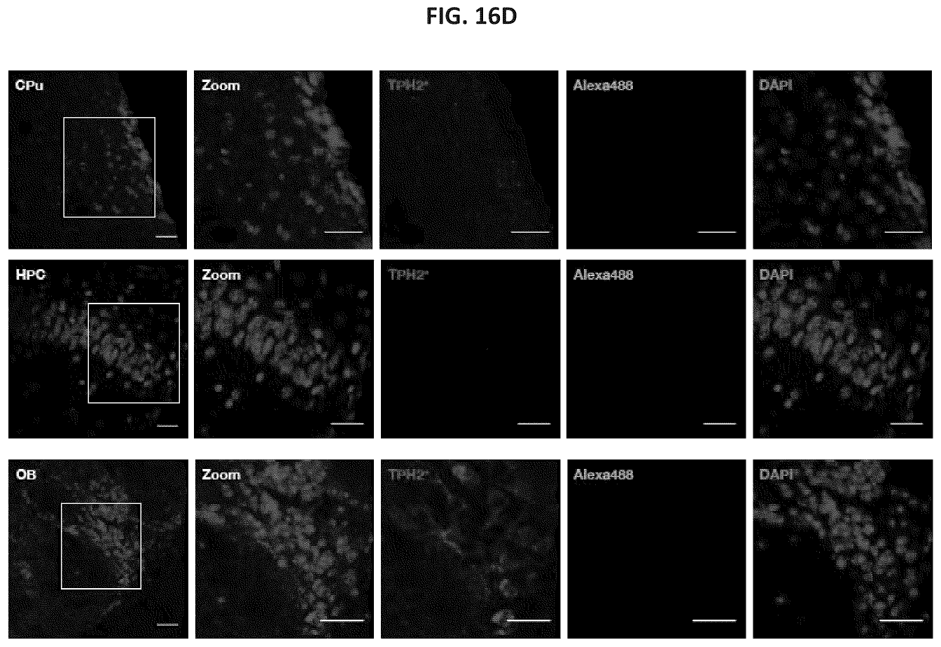

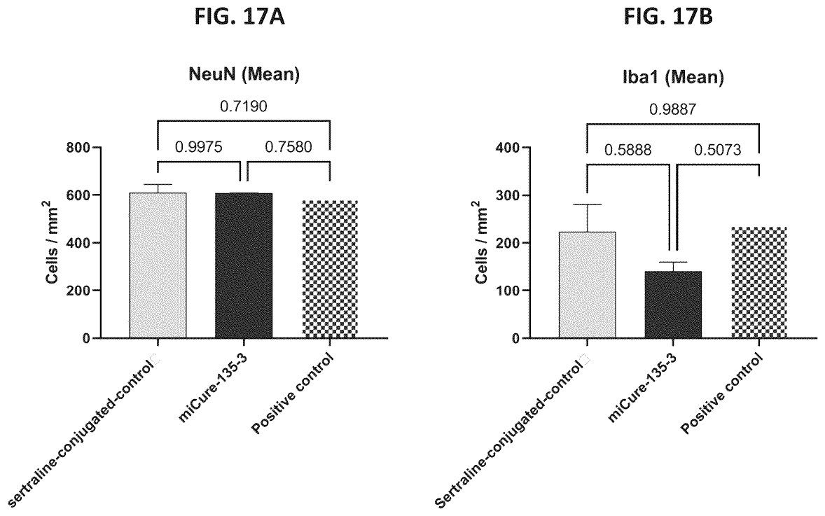

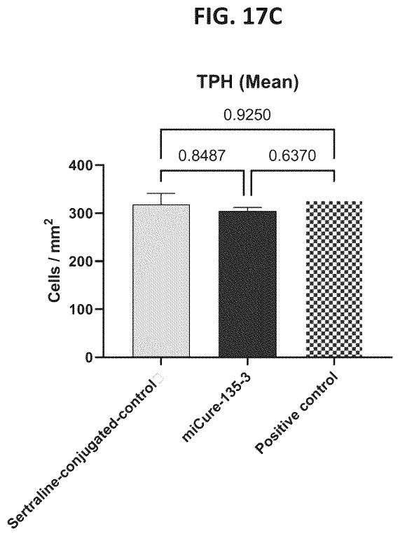

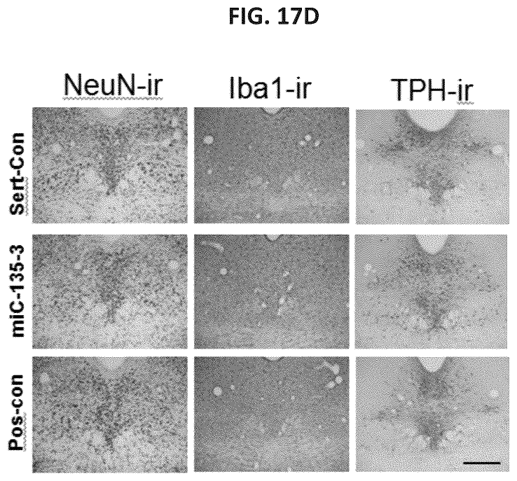

The synthesis was carried out while employing a typical experimental procedure of solid-phase synthesis on a CPG (Controlled Pore Glass) support. In short, a typical oligonucleotide synthesis preceded through a series of cycles composed of fours steps (deprotection, coupling, capping and oxidation) that were repeated until the 5′ most nucleotide was attached (as depicted in FIG. 1).

(a) Deprotection/Detritylation

The acid-labile 5′-dimethoxytrityl protecting group was cleaved from the base that was anchored to the solid phase support (either start nucleoside or later the growing oligo strand); thereby a free reactive hydroxy function was obtained. As cleavage reagent di- or tri-chlor acetic acid in dichlormethane was used.

(b) Coupling

The free 5′-OH group was now able to react with added phosphoramidite. As a result, both nucleosides were linked by a phosphite bridge. The phosphoramidite had to be activated first using a weak acid (e.g. 1H-tetrazole).

(c) Capping

In order to prevent the remaining free OH-groups (about 1%) from reacting in the synthesis cycle, thus creating non-specific sequences, in the capping step all free reactive groups were blocked using acetylation, thus eliminating them as reactive partners in the further course of the synthesis.

(d) Oxidation

The internucleotide phosphit group that was created in the coupling step was oxidized to its phosphate using iodine solution.

A new synthesis cycle was repeated again from step (a). These reactions were repeated until the desired oligonucleotide sequence has been produced. The cycle could be terminated to result in either a solid-support-bound oligonucleotide carrying a free 5′-OH group or one carrying a protected 5′-OH group (DMT).

Incorporation of Phosphorothioate Bonds:

The difference between the synthesis of a normal oligo with a full phosphodiester backbone and an oligo with a partial or full phosphorothioate backbone was based on the choice of oxidizing agent used in step (d) Oxidation.

A phosphodiester bond was produced by using iodine and water to add a fourth oxygen to the phosphate. A phosphorothioate bond was produced by using Beaucage reagent to add a sulfur to the phosphate. Once the sulfur or the oxygen has been attached to the phosphate, the bond was stabilized and was not affected by the subsequent cycles of chemistry. By switching back and forth between the two oxidizing agents, a chimeric backbone could be constructed.

Sertraline-Conjugated miR-135 Synthesis

The guide and passenger RNA strands were synthesized on an automated synthesizer using the phosphoramidite chemistry. The sequences were elongated using rA^{Bz}; rC^{Ac}; rG^{iBu}; rU and _{2′OMe}A^{Bz}; _{2′OMe}C^{Ac}; _{2′OMe}U phosphoramidite monomers. After the chain elongation, the RNA strands were cleaved from their solid support and deprotected using an ammonium hydroxide and methylamine mixture. The 2′OH positions were deprotected using a solution containing fluorine ions. Then, the RNA strands were purified by anion exchange HPLC.

The Sertraline modified passenger RNA strands were synthesized on an automated synthesizer using the phosphoramidite chemistry. The sequences were elongated using rA^{Bz}; rC^{Ac}; rG^{iBu}; rU and _{2′OMe}A^{Bz}; _{2′OMe}C^{Ac}; _{2′OMe}U phosphoramidite monomers. Before the deprotection step, the C10 N-hydroxysuccimide ester linker was grafted to the 5′ end of the passenger RNA sequence still attached to the solid support. The condensation of the Sertraline-C₆Acyl-NH₂ Ligand with the C10 N-hydroxysuccimide ester linker was carried out in dimethylformamide with 3% of N,N-Diisopropylethylamine for 48 hours at room temperature (FIG. 2). After the conjugation, the Sertraline modified passenger RNA strands were cleaved from their solid support and deprotected using an ammonium hydroxide and methylamine mixture. The 2′OH positions were deprotected using a solution containing fluorine ions. Then, the conjugates RNA strands were purified by reverse phase HPLC.

The guide and passenger RNA strands were quantified by UV spectrometry using the extinction coefficient calculated based on the nearest neighbor method. The duplexes were combining equimolar amount of guide and passenger strands. The annealing was done in water. The duplexes were not further purified.

Oligonucleotide primers, RNA strands and sertraline-conjugated RNA strands and RNA duplexes were prepared by Axolabs, GmbH, following the specifications and designs discussed herein.

Structures:

Naked miR-135 Duplex 1 (Duplex 11 Per Table 1, and Duplex 1 Per Table 6, Below)

SEQ ID NO: 10
^{5′}p UAU GGC UUU UCA UUC CUA UGU Ga^{3′}
SEQ ID NO: 13
^{3′}aUA CCG AAA AGU AAG GAU ACA cu ^{5′}

Sertraline Conjugated miR-135 Duplex 1 (miCure-135-1 Per Table 6, Below)

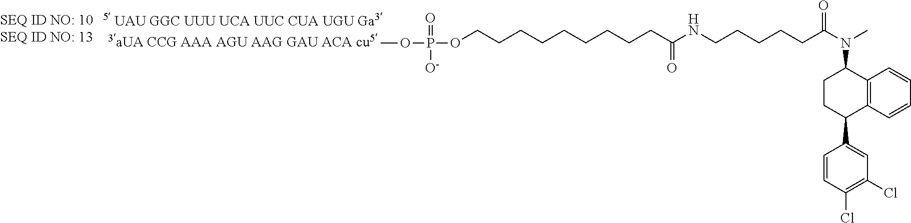 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Sertraline-AcylC6-NHCO-C9-5′miR-135sequence3′-OH

Naked Control (Duplex 16 Per Table 1, and Duplex 8 Per Table 6, Below)

SEQ ID NO: 16
^{5′} p uaU GGC UUU UCA UUC CUA UGU Ga^{3′}
SEQ ID NO: 13
^{3′}aUA CCG AAA AGU AAG GAU ACA cu ^{5′}

Sertraline Conjugated Control (miCure-135-8 Per Table 6, Below)

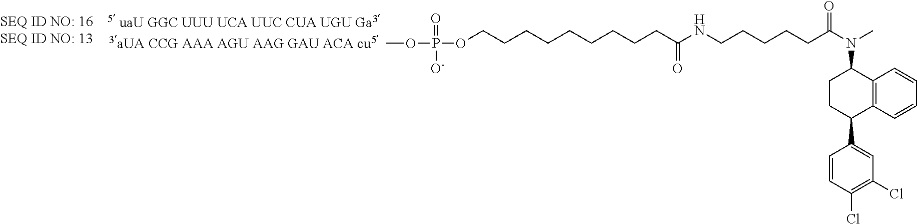 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Naked miR-135 Duplex 2 (Per Table 6, Below)

Guide strand
(SEQ ID NO: 41)
5′-P/UmUAUGGCUUUUCAUUCCUAUGUGa
Passenger strand
(SEQ ID NO: 13)
5′-ucACAUAGGAAUGAAAAGCCAUa

Sertraline Conjugated miR-135 Duplex 2 (miCure-135-2 Per Table 6, Below)

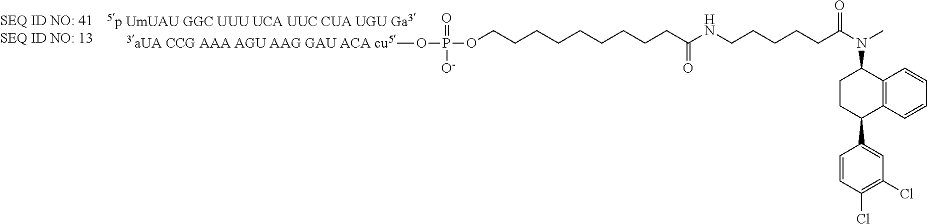 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Naked miR-135 Duplex 3 (per Table 6, below)

Guide strand
(SEQ ID NO: 42)
5′-P/UmUAUGGCUUUUCAUUCCUAUGUGaAmsAm
Passenger strand
(SEQ ID NO: 13)
5′-ucACAUAGGAAUGAAAAGCCAUa

Sertraline Conjugated miR-135 Duplex 3 (miCure-135-3 Per Table 6, Below)

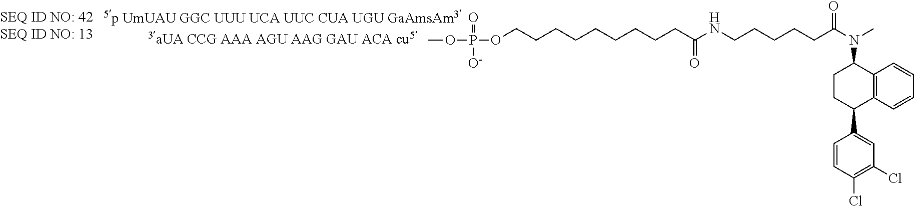 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Naked miR-135 Duplex 9 (Per Table 6, Below)

Guide strand
(SEQ ID NO: 10)
5′-P/UAUGGCUUUUCAUUCCUAUGUGa
Passenger strand
(SEQ ID NO: 47)
5′-uscsACAUAGGAAUGAAAAGCCAsUsa

Sertraline Conjugated miR-135 Duplex 9 (miCure-135-9 Per Table 6, Below)

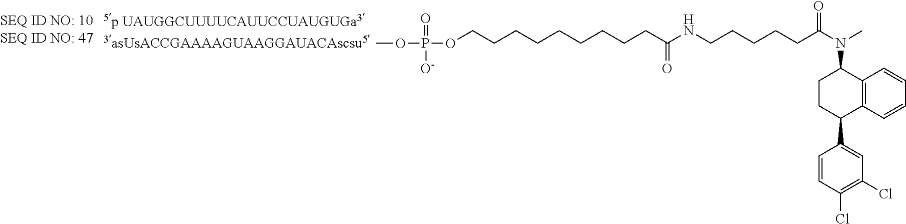 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Naked miR-135 Duplex 10 (Per Table 6, Below)

Guide strand
(SEQ ID NO: 41)
5′-P/UmUAUGGCUUUUCAUUCCUAUGUGa
Passenger strand
(SEQ ID NO: 47)
5′-uscsACAUAGGAAUGAAAAGCCAsUsa

Sertraline Conjugated miR-135 Duplex 10 (miCure-135-10, Per Table 6, Below)

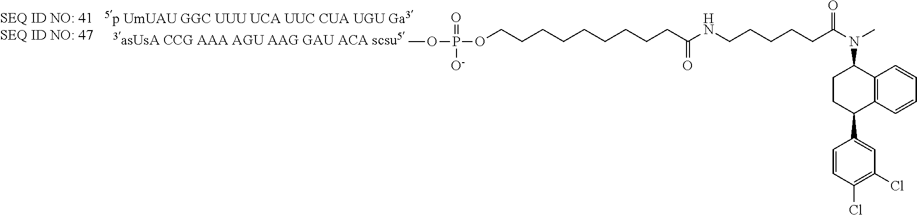 [SMILES: COP(=O)([O-])OCCCCCCCCCC(=O)NCCCCCC(=O)N(C)[C@@H]1CC[C@H](c2ccc(Cl)c(Cl)c2)c2ccccc21]

Naked miR-135 Duplex 11 (Per Table 6, Below)

Guide strand
(SEQ ID NO: 42)
5′-P/UmUAUGGCUUUUCAUUCCUAUGUGaAmsAm
Passenger strand
(SEQ ID NO: 47)
5′-uscsACAUAGGAAUGAAAAGCCAsUsa

Sertraline Conjugated miR-135 Duplex 11 (miCure-135-11 Per Table 6, Below)

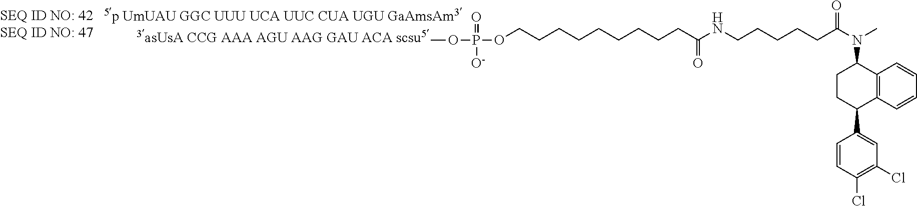 [SMILES: COP(=O)([O-])OCCCCCCCCCC(=O)NCCCCCC(=O)N(C)[C@@H]1CC[C@H](c2ccc(Cl)c(Cl)c2)c2ccccc21]

For all duplexes:
High case letters (e.g. N, A, U, C, G): RNA
Lower-case letters (e.g. a, u, c, g): 2′-O-Me modification
Um: 2′-O-MOE-5′-Me Uracil modification
Am: 2′-O-MOE Adenine modification
Lower-case ‘s’: phosphorothioate. No indication means a normal phosphodiester bond.
P: phosphate
Underscore: 2′-fluoro, i.e. 2′-F
(C10): Carboxymodifier C10
(Glen Research: 10-1935)

Intranasal Administration

Mice were slightly anesthetized by 2% isoflurane inhalation and placed in a supine position. A 5 μl drop of conjugated control or conjugated miR-135 was applied alternatively to each nostril once daily. A total of 10 μl of solution containing 166 μg was delivered.

Intracerebral Injection

For stereotaxic surgery and compound delivery, a computer-guided sterotaxic instrument and a motorized nanoinjector were used (Angle Two™ Stereotaxic Instrument, myNeurolab). Mice were placed on a stereotaxic apparatus under general anesthesia, and the lentiviral preparation was delivered to coordinates determined as defined by the Franklin and Paxinos atlas to the DR: ML 1 mm; AP—4.5 mm; DV −4.2 mm in 200 tilt. Injections were performed at a rate of 0.2 l/1 min.

Intracerebroventricular Chronic Administration

The right lateral ventricle was stereotaxically perforated with brain infusion kit 3 (ALZET, DURECT Corporation, Cupertino, CA; coordinates from the bregma: posterior, −0.7 mm; lateral, −1.5 mm; ventral, −2.0 mm). The infusion cannula was connected to a mini-osmotic pump (ALZET; pump model 1007D) that was implanted subcutaneously in the animal's back, just behind the scapula. Osmotic pumps were primed for approximately 8 hours prior to implantation and were filled with duplex/control (0.5 nmol/day). Once implanted, the pumps continually infused the compound or negative control for 7 days at a rate of 0.5 μl/h.

Intracerebral Microdialysis

Extracellular 5-HT concentration was measured by in vivo microdialysis. Briefly, one concentric dialysis probe (cuprophan; 1 mm-long) was implanted in mPFC (AP, 2.2; ML, −0.2; DV, −3.4) of pentobarbital-anaesthetized mice. Experiments were performed 48-72 hours after surgery. 1 mM citalopram (SSRI; Lundbeck A/S, Valby, Copenhagen) was added to artificial cerebrospinal fluid. The artificial cerebrospinal fluid was pumped at 6 μl min⁻¹ (WPI model sp220i) and 6 minute samples were collected. While collecting the 8^{th} fraction tail suspension test was performed. 5-HT concentrations were analyzed by high-performance liquid chromatography-amperometric detection (Hewlett-Packard 1049; b0.6V, Palo Alto, CA, USA) with detection limits of 1.5 fmol sample. Baseline 5-HT levels were calculated as the average of the four pre-drug samples. Correct probe placement was verified using cresyl-violet staining.

Behavioral Assessments

All behavioral assessments were performed during the dark phase following habituation to the test room for 2 hours before each test. The experimenter running the tests was blind to the mice groups.

Dark-Light Transfer (DLT) test: The DLT test apparatus consists of a polyvinyl chloride box divided into a black dark compartment (14×27×26 cm) and a connected white 1200 lux illuminated light compartment (30×27×26 cm). During the 5 minute test, the time spent in the light compartment, the distance traveled in light area, and the number of light-dark transitions were quantified with a video tracking system (VideoMot2; TSE Systems, Bad Homburg, Germany).

Tail suspension test: Mice were suspended 30 cm above the bench by adhesive tape placed approximately 1 cm from the tip of the tail. Mice were monitored and recorded using a video camera system (Smart, Panlab), and the time spent immobile was recorded for 6 minutes.

8-OH-DPAT-Induced Hypothermia

Body temperature was measured intrarectally using a lubricated probe inserted B2 cm and a digital thermometer (AZ9882, Panlab, Barcelona, Spain). Mice were singly housed in clean cages for 20 minutes before measurements, and then two baseline temperature measurements were taken. After 10 minutes, animals received 8-OH-DPAT 1 mg per kg i.p., and body temperature was recorded every 15 minutes for a total of 120 minutes. Data are presented as a change from the average baseline measurement.

In the experiments examining the effect of the different miR-135 mimetics (as described in the ‘results’ section below), body temperature was measured using a microchip. The mice were subcutaneously implanted with programmable subcutaneous microchip transponders (IPTT-300 Extended Accuracy Calibration; Bio Medic Data Systems, Seaford, DE) following the manufacturer's instructions. In short, this procedure involved a quick insertion of a large-bore needle delivery device containing the microchip, and depression of the plunger on the device that expelled the microchip from the delivery device. Temperature measurements from the microchip transponders were obtained using a compatible reader (catalog no. WRS6007, model IPTT-300, Bio Medic Data Systems). The reader was held at a distance of 5 to 6 cm from the back of the mice as instructed by the manufacturer. An audible beep (after 1 to 3 seconds) signaled completion of the reading, and the displayed temperature was recorded. Replicate temperatures were obtained by taking two readings in succession.

Tissue Preparation for In Situ Hybridization and Receptor Autoradiography

Mice were killed by pentobarbital overdose and the brains rapidly removed, frozen on dry ice and stored at −20° C. Tissue sections, 14-mm thick, were cut using a microtome-cryostat (HM500 OM, Microm, Walldorf, Germany), thaw-mounted onto 3-amino-propyltriethoxysilane-coated slides (Sigma-Aldrich, Madrid, Spain) and kept at −20° C. until use.

Receptor Autoradiography

The autoradiographic binding assays for 5-HT1A and 5-HT1B receptors and serotonin transporter (SERT) were performed using the following radioligands: (a) [³H]-8-OH-DPAT (233 Cimmol⁻¹), (b) [¹²⁵I]-cyanopindolol (2200 Cimmol⁻¹) and (c) [3H]-citalopram (70 Cimmol⁻¹), respectively (Amersham-GE Healthcare, Barcelona, Spain and Perkin-Elmer, Madrid, Spain). 8-OH-DPAT, isoproterenol, pargyline and 5-HT were from Sigma-Aldrich, and fluoxetine was from Tocris. Experimental conditions are summarized in Table 5, below.

TABLE 5
Summary of the conditions for labeling serotonin receptors and transporter

| | | | | | | | Washing | | |
|---|---|---|---|---|---|---|---|---|---|
| | | | | Pre- | | Incubation | protocol | | |
| | | Ligand | | incubation | Incubation | protocol | (I.c.b., | Exposure | |
| Protein | Ligand | (nM) | Buffer | (RT, min) | buffer | (RT, min) | min) | time | Blank |
| 5-HT ₁ₐR | [³H] | 1.0 | A | 30 | A + 10 | 60 | 2 × 5 | 60 | 10 μM |
| | 8-OH- | | | | μM | | | days | serotonin |
| | DPAT | | | | pargyline | | | | |
| 5-HT _{1B}R | [¹²⁵I]- | 0.1 | B | 10 | B + 100 | 120 | 2 × 15 | 1 | 12 μM |
| | Cyanopin- | | | | μM | | | day | serotonin |
| | dolol | | | | 8-OH- | | | | |
| | | | | | DPAT + | | | | |
| | | | | | 30 nM | | | | |
| | | | | | isopro- | | | | |
| | | | | | terenol | | | | |
| SERT | [³H] | 1.5 | C | 15 | C | 60 | 2 × 10 | 45 | 1 μM |
| | citalopram | | | | | | | days | fluoxetine |

Tissues were exposed to Biomax® MR film (Kodak) together with ³H-Microscales standards (Amersham-GE Health-care). All experimental and control brains within a group were processed in duplicate and exposed to films as a batch.

Films were analyzed by microdensitometry using a computer-assisted image analyzer (AIS, Imaging Research, St Catherines, Ontario, Canada). 5-HT1AR mRNA and 5-HT1B binding sites in selected brain regions were measured in the respective autoradio-grams to obtain relative optical densities. For 5-HT1AR and SERT binding, the system was calibrated with ³H-Microscales standards to obtain fmol mg⁻¹ protein equivalents from relative optical densities data. AIS system was also used to acquire pseudocolor images. Black and white photographs were taken from autoradiograms using a Wild 420 microscope (Leica, Heerbrugg, Germany) equipped with a Nikon DXM1200 F digital camera and ACT-1 Nikon soft-ware, Soft Imaging System Gmbh, Munster, Germany. Images were processed with Photoshop (Adobe Systems, Mountain View) by using identical values for contrast and brightness.

Peripheral Blood Mononuclear Cells (PBMCs) Study

The study was performed by Axolabs www(dot)axolabs(dot)com). In short, human PBMCs were isolation from buffy coats of healthy donors (obtained from the Blood Bank Suhl, Germany, Institute for Transfusion Medicine). Buffy coat, at a volume of about 28-32 ml, about 19 hours after blood donation, and delivered sterile in a sealed infusion bag were used. Isolation of human PBMCs (huPBMCs) was carried out using Ficoll gradient centrifugation.

huPMBCs were treated with the different mimetics (as described in the ‘results’ section below) by transfection or direct incubation (at three concentrations for 24 hours). Transfection was carried out using Lipofectamine2000. Supernatants were analyzed for the presence of cytokines using multiplex assays (U-Plex®) run on the meso scale discovery (MSD) platform. This entire procedure was carried out by Axolabs (www(dot)axolabs(dot)com).

Corticosterone—Chronic Restraint Stress Model

For the Chronic Restraint stress protocol, mice were isolated and handled 3 days before the start of the experiment. Along 28 days, once a day, mice were gently introduced in holed 50 mL Falcon tubes, fixed in a plastic tray, and left for 2 hours in a dark, quiet room. Control mice were not isolated and only handled daily for 1 minute each.

Corticosterone (Cortico, Sigma-Aldrich, Madrid, Spain) was dissolved in commercial mineral water and brought to a pH 7.0-7.4 with HCl. Isolated mice were presented with Cortico solution for the 28 days of Chronic Restraint stress protocol, at 30 μg ml⁻¹ for 15 days, followed by 15 μg ml⁻¹ for 3 days and 7.5 μg ml⁻¹ for 10 days. Cortico solutions were no more than 3 days old and maintained in opaque bottles to protect them from light. Control mice were presented only with mineral water.

Chronic Social Defeat Stress

Nine-week-old C57BL/6J mice were subjected to a chronic social defeat stress (CSDS) protocol as previously described [Krishnan V. et al., Cell (2007) 131:391-404]. Briefly, the mice were placed in the home cage of an aggressive ICR (CD1) outbred mouse (Harlan) and were allowed to physically interact for 5 minutes. During this time, the ICR mouse attacked the intruder mouse and the intruder displayed subordinate posturing. A perforated clear Plexiglas® divider was then placed between the animals, and the mice remained in the same cage for 24 hours allowing sensory contact. The procedure was then repeated with an unfamiliar ICR mouse for each of the 10 consecutive days. Control mice were housed in the same room as the social defeat mice but were taken out of the room during the 5 minute interaction with the ICR mouse. Control mice were handled daily and housed two per cage separated by a perforated clear Plexiglas® divider.

Immunohistochemistry

Mice were anaesthetized with pentobarbital and transcardially perfused with 4% paraformaldehyde (PFA) in sodium-phosphate buffer (pH 7.4). Brains were extracted, post-fixed 24 hours at 4° C. in PFA, and placed in gradient sucrose solution 10-30% for 3 days at 4° C. After cryopreservation, serial 30 μm-thick sections were cut to obtain prefrontal cortex (PFC), caudate putamen (CPu), hippocampus (HPC), and dorsal raphe nuclei (DRN). Brain sections were washed and incubated in a 1×PBS/Triton 0.2% solution containing normal serum from secondary antibody host. Primary antibodies for NeuN (anti-NeuN 1:1000; ref: MAB377, Millipore), Iba1 (anti-Iba1 1:1000; ref: 019-197741, Wako), and TPH (anti-TPH1 1:2500; ref: AB1541, Millipore) were used. Briefly, primary antibodies were incubated overnight at 4° C., followed by incubation with the corresponding biotinylated anti-mouse IgG1 (1:200; ref: A-10519, Life Technologies) for anti-NeuN, biotinylated anti-rabbit (1:200; ref: BA-1000, Vector Laboratories) for anti-Iba1, and biotinylated anti-sheep (1:200; ref BA-6000, Vector Laboratories) for anti-TPH1. The color reaction was performed by incubation with diaminobenzidine tetrahydrochloride (DAB) (ref: 18865.02, Quimigen) solution. Sections were mounted and embedded in Entellan (Electron Microscopy Sciences). The number of NeuN-positive cells, Iba1-positive cells and TPH-positive cells in the DRN were assessed in sections corresponding to different antero-posterior levels −4.24 mm to −4.84 mm from the bregma using ImageJ software (v1.51s, NIH, Bethesda. MD, USA). All labeled cells with its nucleus within the counting frame were counted in three consecutive DRN sections.

Immunofluorescence

Mice were anaesthetized with pentobarbital and transcardially perfused with 4% paraformaldehyde (PFA) in sodium-phosphate buffer (pH 7.4). Brains were extracted, post-fixed 24 hours at 4° C. in PFA, and placed in gradient sucrose solution 10-30% for 3 days at 4° C. After cryopreservation, serial 30 μm-thick sections were cut to obtain olfactory bulb (OB), CPu, HPC and DRN. Brain sections were washed and incubated in a 1×PBS/Triton 0.2% solution containing normal serum from secondary antibody host. Primary antibodies Alexa488 (anti-Alexa488 1:1000; ref.: A11094, Invitrogen) and TPH (anti-TPH 1:1541; ref.: ab1541, Abcam) were used. Briefly, primary antibodies were incubated overnight at 4° C., rinsed and treated with secondary antibodies A555-anti-sheep (1:500; ref.: A-21436, Life Technologies) and Alexa488-anti-rabbit (1:500; ref.: A-21206, Life Technologies) for 120 minutes. Nuclei were stained with Hoechst (1:10.000, ref.: H3570, Life Technologies).

Intracellular localization of Alexa488-conjugated-miR-135 in TPH⁺ neurons was observed and imaged using an inverted Nikon Eclipse Ti2-E microscope (Nikon Instruments) attached to the spinning disk unit Andor Dragonfly (Oxford Instruments). For all experiments an oil-immersion objective (Plan Apochromat Lambda blue 60×, numerical aperture (NA) 1.4, oil) was used. Samples were excited with 405 nm, 488 nm and 561 nm laser diodes. The beam was coupled into a multimode fiber going through the Andor Borealis unit reshaping the beam from a Gaussian profile to a homogenous flat top. From there it was passed through the 40 μm pinhole disk. Tissue sections were imaged on a high resolution scientific complementary metal oxide semiconductor (sCMOS) camera (Zyla 4.2, 2.0 Andor, Oxford Instruments Company). Fusion software (Andor, Oxford Instruments) was used for acquisition and ImageJ/Fiji (1.51s, open source software) was used for image processing.

Western Blot

Tissue samples of OB, PFC, CPu, HPC, DRN and cerebellum (Cb) were dissected from brain slices and homogenised in RIPA buffer (150 mM NaCl, 1% Triton X-100, 0.5% Sodium deoxycolate, 5 mM EDTA, 0.1% SDS, 50 mM Tris, pH 8.0) with protease and phosphatase inhibitors. Proteins were quantified using Pierce™ BCA Protein Assay Kit (Thermo Fisher Scientific). Protein lysate (10-15 μg) was separated using 4-15% SDS-PAGE and electro-transferred onto a nitrocellulose membrane. Protein blots were probed with primary antibodies against SERT (1:1000, ref.: ab130130, Abcam) and 5HT1AR (1:1000, ref.: ab85165, Abcam), and anti-O-actin (1:50000, ref.: A3854, Sigma-Aldrich) as loading control, followed by incubation with the corresponding HRP-conjugated anti-goat (1:20000, ref.: P0449, Dako) for SERT, and HRP-conjugated anti-rabbit IgG (1:10000; ref.: NA934, GE Healthcare Life) for 5HT1AR. Detection was done by chemiluminescence using SuperSignal™ Chemiluminescence ECL substrate kit (Thermo Fisher Scientific), and pictures were taken using ChemiDoc™ Imaging System (Bio-Rad). Images were analysed using ImageLab™ software (BioRad).

Statistical Analysis

Data are expressed as means±s.e.m. Data were analyzed with Student's t-test, one- or two-way analysis of variance, as appropriate, followed by post-hoc test (Newman-Keuls). The level of significance was set at P<0.05 (two tailed).

### Example 1

### Design, Synthesis and Validation of miR-135 Mimetic Oligos In-Vitro

miR-135 mimetics (oligos) were designed to mimic the antidepressant effects demonstrated using overexpression of endogenous miR-135 in transgenic mouse model and viral manipulation, in the mouse 5HT neurons or DRN, respectively. The oligos (miR-135 mimetics) were designed based on the endogenous miR-135 with few modifications aimed to improve stability and cell penetration. The altered oligonucleotides were synthesized by an oligonucleotide manufacturing company called BioSpring (www(dot)biospring(dot)de) using conventional chemistry and each oligo was used as a duplex to improve stability. Seventeen different mimics (see Table 1) were designed, synthesized and screened in-vitro, using luciferase assay, for their effects on the validates Htr1a and Slc6a4 target transcripts. For the luciferase assay screening the human cell line HEK293T and the transfection reagent, lipofectamin 2000 (Thermo Fisher Scientific) were used.

Duplex 11 was found to be the most potent miR-135 mimetic oligo, affecting significantly both 5HTR1A and SLC6a4 levels (using 3′UTR luciferase constructs; FIGS. 3A-B).

The design of duplex 11 mimetic of miR-135 was as follows:

Guide strand
(SEQ ID NO: 10)
5Ph/UAUGGCUUUUCAUUCCUAUGUGa
Passenger strand
(SEQ ID NO: 13)
ucACAUAGGAAUGAAAAGCCAUa

Lower-case letters (e.g. a, u, c, g): 2′-O-Me modification
Ph=phosphate, 5 indicates that this is the 5 prime end of the sequence

The mimetic that was identified to consistently reduce the target transcripts (Htr1a and Slc6a4) across preparations was further used for the in-vivo experiments.

TABLE 1
miR-135 mimetics (oligos) tested

| Duplex id | | oligo id | Oligo sequence | Comments |
|---|---|---|---|---|
| DUP-001 | Guide | OLG-135-001 | 5′Ph/UAUGGCUUUUUAUUCCUAUGUGa | minimal mod |
| | | | (SEQ ID NO: 1) | |
| | Passenger | OLG-135-002 | uaUAGGGAUUGGAGCCGUGGCg | minimal mod |
| | | | (SEQ ID NO: 2) | |
| DUP-002 | Guide | OLG-135-001 | 5′Ph/UAUGGCUUUUUAUUCCUAUGUGa | minimal mod |
| | | | (SEQ ID NO: 1) | |
| | Passenger | OLG-135-003 | ucAUAUAGGGAUUGGAGCCGUg | minimal mod |
| | | | (SEQ ID NO: 3) | and cut overhangs |
| DUP-003 | Guide | OLG-135-001 | 5′Ph/UAUGGCUUUUUAUUCCUAUGUGa | minimal mod |
| | | | (SEQ ID NO: 1) | |
| | Passenger | OLG-135-004 | ucACAUAGGAAUAAAAAGCCAUa | minimal mod |
| | | | (SEQ ID NO: 4) | and complete match |
| DUP-004 | Guide | OLG-135-005 | 5′Ph/UAUGGCUUUuuAUUCCUAUGUGa | bulge protection |
| | | | (SEQ ID NO: 5) | |
| | Passenger | OLG-135-002 | uaUAGGGAUUGGAGCCGUGGCg | minimal mod |
| | | | (SEQ ID NO: 2) | |
| DUP-005 | Guide | OLG-135-001 | 5′Ph/UAUGGCUUUUUAUUCCUAUGUGa | minimal mod |
| | | | (SEQ ID NO: 1) | |
| | Passenger | OLG-135-006 | uaUAGGGAUuGGAGCCGUGGCg | bulge protection |
| | | | (SEQ ID NO: 6) | |
| DUP-006 | Guide | OLG-135-005 | 5′Ph/UAUGGCUUUuuAUUCCUAUGUGa | bulge protection |
| | | | (SEQ ID NO: 5) | |
| | Passenger | OLG-135-006 | uaUAGGGAUuGGAGCCGUGGCg | bulge protection |
| | | | (SEQ ID NO: 6) | |
| DUP-007 | Guide | OLG-135-007 | 5′Ph/uaUgGCUUUUUAUUCCUAUGUGa | miR seq seed block |
| | | | (SEQ ID NO: 7) | |
| | Passenger | OLG-135-002 | uaUAGGGAUUGGAGCCGUGGCg | minimal mod |
| | | | (SEQ ID NO: 2) | |
| DUP-008 | Guide | OLG-135-008 | 5′Ph/uaAuUUAAGCUUCUUUGUUCUGg | scamble seq |
| | | | (SEQ ID NO: 8) | seed block |
| | Passenger | OLG-135-009 | cCAGAACAAAGAAGCUUAAAUUa | scramble pass |
| | | | (SEQ ID NO: 9) | |
| DUP-009 | Guide | OLG-135-010 | 5′Ph/UAUGGCUUUUCAUUCCUAUGUGa | minimal mod |
| | | | (SEQ ID NO: 10) | |
| | Passenger | OLG-135-011 | auGUAGGGCUAAAAGCCAUGGg | minimal mod |
| | | | (SEQ ID NO: 11) | |
| DUP-010 | Guide | OLG-135-010 | 5′Ph/UAUGGCUUUUCAUUCCUAUGUGa | minimal mod |
| | | | (SEQ ID NO: 10) | |
| | Passenger | OLG-135-012 | ucAUGUAGGGCUAAAAGCCAUG | minimal mod |
| | | | (SEQ ID NO: 12) | and cut overhangs |
| DUP-011 | Guide | OLG-135-010 | 5′Ph/UAUGGCUUUUCAUUCCUAUGUGa | minimal mod |
| | | | (SEQ ID NO: 10) | |
| | Passenger | OLG-135-013 | ucACAUAGGAAUGAAAAGCCAUa | minimal mod |
| | | | (SEQ ID NO: 13) | and complete match |
| DUP-012 | Guide | OLG-135-014 | 5′Ph/UAUGGCUUUUcauUCCUAUGUGa | bulge protection |
| | | | (SEQ ID NO: 14) | |
| | Passenger | OLG-135-011 | auGUAGGGCUAAAAGCCAUGGg | minimal mod |
| | | | (SEQ ID NO: 11) | |
| DUP-013 | Guide | OLG-135-010 | 5′Ph/UAUGGCUUUUCAUUCCUAUGUGa | minimal mod |
| | | | (SEQ ID NO: 10) | |
| | Passenger | OLG-135-015 | auGUAGGGcuAAAAGCCAUGGg | bulge protection |
| | | | (SEQ ID NO: 15) | |
| DUP-014 | Guide | OLG-135-014 | 5′Ph/UAUGGCUUUUcauUCCUAUGUGa | bulge protection |
| | | | (SEQ ID NO: 14) | |
| | Passenger | OLG-135-015 | auGUAGGGcuAAAAGCCAUGGg | bulge protection |
| | | | (SEQ ID NO: 15) | |
| DUP-015 | Guide | OLG-135-016 | 5′Ph/uaUgGCUUUUCAUUCCUAUGUGa | miR seq seed block |
| | | | (SEQ ID NO: 16) | |
| | Passenger | OLG-135-011 | auGUAGGGCUAAAAGCCAUGGg | minimal mod |
| | | | (SEQ ID NO: 11) | |
| DUP-016 | Guide | OLG-135-016 | 5′Ph/uaUgGCUUUUCAUUCCUAUGUGa | |
| | | | (SEQ ID NO: 16) | |
| | Passenger | OLG-135-013 | ucACAUAGGAAUGAAAAGCCAUa | |
| | | | (SEQ ID NO: 13) | |
| DUP-017 | Guide | OLG-135-007 | 5′Ph/uaUgGCUUUUUAUUCCUAUGUGa | |
| | | | (SEQ ID NO: 7) | |
| | Passenger | OLG-135-004 | ucACAUAGGAAUAAAAAGCCAUa | |
| | | | (SEQ ID NO: 4) | |

### Example 2

### Effects of miR-135 Mimetics on Serotonergic Function In-Vivo

To examine the effect of miR-135 mimetics on the serotonergic system in-vivo, the miR-135 mimetic (duplex 11) was delivered directly into the dorsal raphe nucleus (DRN) of wild type mice using stereotactic surgery. The naked (i.e. unconjugated) mimetic was administrated acutely (100 μg) and the physiological consequences of 5-HT1A-auto receptor (HTR1a) silencing were examined using the hypothermia response induced by the selective 5-HT1AR agonist, 8-OH-DPAT, an effect mediated exclusively by pre-synaptic 5-HT1AR in mice. miR-135 mimetic (duplex 11)-treated mice did not show 8-OH-DPAT-induce hypothermia, while both control groups: mice treated with artificial cerebrospinal fluid (aCSF) or mice treated with the control miR (i.e. naked control), displayed the expected hypothermic response (FIGS. 4A-D). No difference in baseline temperature was found between the groups (FIG. 4E). The kinetics of this effect was demonstrated at 4 different time points (24, 48, 72 and 96 hrs) following the acute administration (FIGS. 4A-D).

### Example 3

In-vivo effects of sertraline-conjugated miR-135 mimetic on serotonergic function Following in-vitro and in-vivo validation of miR-135 mimetic efficiency, a previously reported approach was used (Ferrés-Coy et al., Mol. Psychiatr. (2016) 21(3): 328-38) for non-invasive delivery of oligonucleotide to the brain using sertraline-conjugated intranasal administration. Ferrés-Coy et al., (2016, supra) previously reported that intranasal administration of a sertraline-conjugated small interfering RNA (siRNA) silenced SERT expression/function in mice. After crossing the permeable olfactory epithelium, the sertraline-conjugated siRNA was internalized and transported to serotonin cell bodies by deep Rab-7-associated endomembrane vesicles.

Modified miR-135 mimetic and control oligo were conjugated to a non-functional sertraline (purchased from NEDKEN SOLUTIONS, S.L., Barcelona, Spain) and were administrated acutely into the dorsal raphe nucleus (DRN) of naïve mice. Mice treated with the sertraline-conjugated miR-135 mimetic Duplex 11 (termed miCure-135-1, as set forth in SEQ ID Nos: 10 and 13) at a dose of 100 μg, did not show hypothermia response following 8-OH-DPAT administration. This effect lasted up to 7 days following the acute administration (FIGS. 5A-D).

### Example 4

### Acute Intracerebral Administration of Sertraline-Conjugated miR-135 Mimetic (30 μg) Silences 5HT1a and SERT and Evokes Anti-Depressant-Like Responses

To further explore the efficacy of sertraline-conjugated miR-135 in downregulating the expression of genes in the serotonergic system that were previously demonstrated to be directly regulated by miR-135, a lower dose of miCure-135-1 (30 μg) was acutely administrated into DRN of adult mice. First, as was done with the higher dose (100 μg), the physiological consequences of 5-HT1A-auto receptor (HTR1a) silencing were examined using the hypothermia response induced by 8-OH-DPAT. The results indicated that a single administration of miCure-135-1 in a dose of 30 μg was sufficient to abolish the hypothermia induced by selective HTR1a agonist, up to 7 days following the treatment and without altering the baseline temperature (FIGS. 6A-E).

Next, a microdialysis probe was fixated into the medial prefrontal cortex (mPFC) of mice which enabled the collection of CSF in real time and measure subsequently the 5-HT levels using liquid chromatography. Mice were single caged and 15 fractions of 6 minutes each were collected. The 8^{th} fraction was collected during a tail suspension test (a test used for assessing depression-like behavior in mice). The results revealed that mice treated with miCure-135-1 showed a significant reduction in immobility time compered to control group (FIG. 6G) which indicates the anti-depressive like effect of sertraline-conjugated miR-135. Remarkably, coincides with the behavioral results, mPFC 5-HT levels of the treated mice were significantly higher (FIG. 6F), suggesting a better coping mechanism of sertraline-conjugated miR-135 treated mice. Next, the present inventor evaluated whether sertraline-conjugated miR-135 could silence the SERT gene. Histological examination at 1-4 days post-administration revealed that SERT binding densities were significantly reduced in the dorsal raphe 24 hours and 96 hours post injection (75% of control; FIG. 6H).

### Example 5

### Acute Intranasal Administration of Sertraline-Conjugated miR-135 Mimetic (166 μg) Silences 5HT1a and Evokes Anti-Depressant/Anxiolytic-Like Responses

The potential of miCure-135-1 to serve as an anti-depressant following a non-invasive delivery method was examined using intranasal delivery where anesthetized mice received 5 μl of miCure-135-1 in each nostril and a total of 166 μg. 8-OH-DPAT induced hypothermia was examined 5 days following treatment and revealed that the miCure-135-1 treated mice group showed scientifically lower hypothermic response (FIG. 7A), a physiological consequence of HTR1a silencing. On top of that, an anti-depressive like effect was shown by decreased immobility time at the tail suspension test of miCure-135-1-treated mice compared to controls (FIG. 7B) and anxiolytic effect as treated mice spent more time in the lit compartment of the dark/light transfer test compared to controls (FIG. 7C).

### Example 6

### Design, Synthesis and Validation of 16 Advanced Chemically Modified miR-135 Mimetic Oligos In-Vitro

Additional miR-135 mimetics (oligos) were designed based on the endogenous miR-135 as described above (see Table 1, above), but comprised advanced chemical modifications aimed to improve stability and reduce innate immune toxicity without leading to reduced potency.

Sixteen different mimics (see FIG. 8 and Table 6, below) were designed and synthesized using conventional chemistry (as described in the ‘general materials and experimental procedures’ section above), and each oligo was used as a duplex to improve stability. The sixteen different miR-135 mimetics were screened in-vitro, using luciferase assay, for their effects on the validates Htr1a and Slc6a4 target transcripts. For the luciferase assay screening the human cell line HEK293T and the transfection reagent, lipofectamin 2000 (Thermo Fisher Scientific) were used.

miCure-135-1, miCure-135-2, miCure-135-3, miCure-135-9, miCure-135-10 and miCure-135-11 were found to be the most potent miR-135 mimetic oligos, affecting significantly both 5HTR1A and SLC6a4 levels (using 3′UTR luciferase constructs; FIGS. 9A-B).

The mimetics that were identified to consistently reduce the target transcripts (Htr1a and Slc6a4) across preparations were further used in additional experiments.

TABLE 6
Additional miR-135 mimetics (oligos) tested

| Naked Duplex | Sertraline-Conjugated | | | |
|---|---|---|---|---|
| No. | Duplex No. | Guide | Passenger | Remarks |
| 1 | miCure-135-1 | Olg-1 | Olg-9 | Original Positive (Previously |
| | | (SEQ ID NO: 10) | (SEQ ID NO: 13) | designated Duplex 11, per Table 1, |
| | | | | above) |
| 2 | miCure-135-2 | Olg-2 | Olg-9 | |
| | | (SEQ ID NO: 41) | (SEQ ID NO: 13) | |
| 3 | miCure-135-3 | Olg-3 | Olg-9 | |
| | | (SEQ ID NO: 42) | (SEQ ID NO: 13) | |
| 4 | miCure-135-4 | Olg-4 | Olg-9 | |
| | | (SEQ ID NO: 43) | (SEQ ID NO: 13) | |
| 5 | miCure-135-5 | Olg-5 | Olg-9 | |
| | | (SEQ ID NO: 44) | (SEQ ID NO: 13) | |
| 6 | miCure-135-6 | Olg-6 | Olg-9 | |
| | | (SEQ ID NO: 45) | (SEQ ID NO: 13) | |
| 7 | miCure-135-7 | Olg-7 | Olg-9 | |
| | | (SEQ ID NO: 46) | (SEQ ID NO: 13) | |
| 8 | miCure-135-8 | Olg-8 | Olg-9 | Original Negative (Previously |
| | | (SEQ ID NO: 16) | (SEQ ID NO: 13) | designated Duplex 16, per Table 1, |
| | | | | above) |
| 9 | miCure-135-9 | Olg-1 | Olg-10 | Original guide |
| | | (SEQ ID NO: 10) | (SEQ ID NO: 47) | |
| 10 | miCure-135-10 | Olg-2 | Olg-10 | |
| | | (SEQ ID NO: 41) | (SEQ ID NO: 47) | |
| 11 | miCure-135-11 | Olg-3 | Olg-10 | |
| | | (SEQ ID NO: 42) | (SEQ ID NO: 47) | |
| 12 | miCure-135-12 | Olg-4 | Olg-10 | |
| | | (SEQ ID NO: 43) | (SEQ ID NO: 47) | |
| 13 | miCure-135-13 | Olg-5 | Olg-10 | |
| | | (SEQ ID NO: 44) | (SEQ ID NO: 47) | |
| 14 | miCure-135-14 | Olg-6 | Olg-10 | |
| | | (SEQ ID NO: 45) | (SEQ ID NO: 47) | |
| 15 | miCure-135-15 | Olg-7 | Olg-10 | |
| | | (SEQ ID NO: 46) | (SEQ ID NO: 47) | |
| 16 | miCure-135-16 | Olg-8 | Olg-10 | Original guide |
| | | (SEQ ID NO: 16) | (SEQ ID NO: 47) | |

### Example 7

### Advanced Chemical Modifications of miR-135 Oligos Changed the Pattern of Cytokine Secretion from PBMCs In-Vitro

Oligonucleotides that the body does not recognize as self typically induce an immune response through the release of pro-inflammatory cytokines. Chemical modifications of oligonucleotides are aimed at increasing potency and have the potential to reduce innate immune activation. In order to test the pattern of immune activation induced by the new modifications, human peripheral blood mononuclear cells (PBMCs) were isolated by Ficoll density gradient centrifugation starting from fresh buffy coats of healthy volunteers (obtained from a blood donation center, as described in the ‘general materials and experimental procedures’ section above). PMBCs were treated with the different mimetics for 24 hours and the supernatant was analyzed for the presence of cytokines using multiplex assays run on the meso scale discovery (MSD).

The results of this assay illustrated that 2 of the new duplexes, miCure-135-2 and miCure-135-3 did not induce secretion of the tested cytokines (FIGS. 10A-E and FIGS. 11A-F, respectively), miR-135-1 showed a modest activation of TNF-alpha (FIG. 10A) and IFN-alpha-2a (FIG. 10B), miR-135-9 induced high secretion of IFN-alpha-2a (FIG. 11A) and low secretion TNF-alpha (FIG. 11B), miR-135-11 induced high secretion of IFN-alpha-2a (FIG. 11A), and low secretion of TNF-alpha (FIG. 11B) and IFN-gamma (FIG. 11C). MiCure-135-10 results revealed a very high secretion and TNF-alpha and IFN-alpha-2a (FIGS. 10A and 10B, respectively). The duplexes that were identified to induce the lowest level of secretion (i.e. miCure-135-2 and miCure-135-3) were further tested in-vivo. It is possible that the relatively high cytokine secretion induced by miCure-135-9, miCure-135-10 and miCure-135-11 relate to the shared passenger strand that consists of two phosphorothioate bonds replacing the normal phosphodiesters at the 5′ end and one at the 3′ end, this is tested.

### Example 8

### Acute DRN Administration of Sertraline Conjugated miR-135 Mimetics Affects Serotonergic Function and Evokes an Anti-Depressant-Like Response

To further examine the effect of miR-135 mimetics on the serotonergic system in-vivo, three miR-135 mimetics conjugated to a non-functional sertraline, i.e. miCure-135-2 (set forth in SEQ ID Nos: 41 and 13), miCure-135-3 (set forth in SEQ ID nos: 42 and 13) and miCure-135-9 (set forth in SEQ ID Nos: 10 and 47), were delivered directly into the dorsal raphe nucleus (DRN) using stereotactic surgery.

miCure-135-2 and miCure-135-9 were administrated acutely (30 μg) and the physiological consequences of 5-HT1A-auto receptor (HTR1a) silencing were examined using the hypothermia response induced by the selective 5-HT1AR agonist, 8-OH-DPAT, an effect mediated exclusively by pre-synaptic 5-HT1AR in mice. As illustrated in FIGS. 12A-D, sertraline-conjugated miR-135-treated mice (of both mimetics miCure-135-2 and miCure-135-9) did not show hypothermia, while both control groups, i.e. mice treated with artificial cerebrospinal fluid (aCSF) and mice treated with the control miR, displayed the expected hypothermic response. No difference in baseline temperature was observed between the groups (FIG. 12E). The kinetics of this effect was demonstrated at three different time points following the acute administration.

Next, the miCure-135-3 and sertraline conjugated control were administrated acutely (100 μg) into the mice DRN and the functional consequences of serotonin transporter (SERT) silencing were examined. To that end, a microdialysis probe was fixated into the medial prefrontal cortex (mPFC) of mice which enabled the collection of CSF in real time and the subsequent measure of 5-HT levels using liquid chromatography. Mice were single caged and 12 fractions of 20 minutes each were collected. Starting from the 7^{th} fraction a local selective serotonin reuptake inhibitor (Citalopram 10 μM) was infused by reverse-dialysis and resulted in an increase of extracellular 5-hydroxytryptamine (5-HT). The increase of the 5-HT levels in the miCure-135-3-treated mice was significantly lower compared with the levels in the control group (FIG. 12F) suggesting that miCure-135-3 lead to a decrease in the available serotonin transporter. The same experimental group, i.e. mice that were injected once to the DRN with 100 μg of miCure-135-3 or conjugated control were tested in the tail suspension test. The results revealed that mice treated with miCure-135-3 showed a significant reduction in immobility time compered to control group (FIG. 12G) which indicates the anti-depressive like effect of sertraline-conjugated miR-135 mimetics (e.g. miCure-135-3).

### Example 9

### Intranasal and Intracerebroventricular Administration of Sertraline-Conjugated miR-135 Mimetics Silences 5HT1a and SERT

The potential of sertraline-conjugated miR-135 mimetics to serve as anti-depressant agents with clinically acceptable methods of drug administration was demonstrated using a non-invasive method of administration (i.e. intranasal). Mice were treated acutely with miCure-135-3 and 48 hours following administration 8-OH-DPAT induced hypothermia was examined. Administration of 200 μg (FIG. 13C) and 100 μg (FIG. 13B) miCure-135-3 significantly reduced the hypothermia response of treated mice compared to control treated mice. Acute intranasal administration of 50 μg of miCure-135-3 showed a clear tendency (p=0.054) towards reduced hypothermia response (FIG. 13A). A similar response was demonstrated following 7 days of intranasal administration of miCure-135-2 (200 μg/day) (FIG. 13D).

The intracerebroventricular (ICV) delivery method was aimed to model intrathecal administration as in both cases the drug is administrated directly into the CSF. In this experiment, a subcutaneous osmotic mini-pump was connected to a cannula that was placed in the mice brain ventricle. miCure-135-3 was delivered constantly to the 2^{nd} brain ventricle in a rate of 200 μg/day for 7 days. A lower reduction in body temperature was observed in the miCure-135-2 treated group compared to control (FIG. 13E). Taken together, the hypothermia results (FIGS. 13A-E) suggest that the sertraline-conjugated miR-135 mimetics successfully reduce the serotonergic auto receptor (5HT1a).

Next, to examine the effect of the sertraline-conjugated miR-135 mimetics on the levels of serotonin transporter (SERT), a microdialysis probe was fixated into the medial prefrontal cortex (mPFC) of mice which enabled the collection of CSF in real time and subsequent measure of the 5-HT levels using liquid chromatography. Mice were single caged and 18 fractions of 20 minutes each were collected. Starting from the 7^{th} fraction a local selective serotonin reuptake inhibitor (Citalopram 10 μM) was infused by reverse-dialysis and resulted in increase of extracellular 5-hydroxytryptamine (5-HT). The increase of the 5-HT levels in the miCure-135-3-treated groups was significantly lower compared with the levels in the control group (FIG. 13F) suggesting that the intranasal administration of sertraline-conjugated miR-135 mimetics (e.g. miCure-135-3) decreases the serotonin transporter in the dorsal raphe nucleus.

### Example 10

### Acute Intranasal Administration of miCure-135-3 Effects the Serotonergic Function and Evokes Anti-Depressant-Like Responses

To further explore the effects of sertraline-conjugated miR-135 mimetics that were administrated in a non-invasive fashion, on the serotonergic functions, miCure-135-3 was delivered to wildtype mice using an intranasal route of administration. In order to evaluate whether sertraline-conjugated miR-135 could reduce the protein levels of SERT and the 5-HT1A-auto receptor (HTR1AR), mice were treated acutely with miCure-135-3 and immunoblotting analysis of samples taken 3 days post-administration revealed that SERT and HTR1Ar protein levels were significantly reduced in the dorsal raphe 72 hours post injection (75% of control for both proteins; FIGS. 14A-D).

Next, to examine the effect of the sertraline-conjugated miR-135 mimetics on the levels of serotonin transporter (SERT), a microdialysis probe was fixated into the medial prefrontal cortex (mPFC) of mice which enabled the collection of CSF in real time and subsequent measure of the 5-HT levels using liquid chromatography. 48 hours post administration, mice were single caged and 12 fractions of 20 minutes each were collected. Starting from the 7^{th} fraction a local selective serotonin reuptake inhibitor (Citalopram 10 μM) was infused by reverse-dialysis and resulted in increase of extracellular 5-hydroxytryptamine (5-HT). The increase of the 5-HT levels in the miCure-135-3-treated groups was significantly lower compared with the levels in the control group (FIG. 14F) suggesting that the intranasal administration of sertraline-conjugated miR-135 mimetics (e.g. miCure-135-3) decreases the serotonin transporter in the dorsal raphe nucleus.

Using the same probes that were fixated into the mPFC, the same group of mice underwent an additional microdialysis experiment aimed to examine the effect of acute intranasal administration of miCure-135-3 on the levels of HTR1AR. 72 hours post administration, CSF was collected in real time in order to measure the 5-HT levels using liquid chromatography. Mice were single caged and 12 fractions of 20 minutes each were collected. On the 6^{th} fraction an i.p. injection of a selective HTR1a agonist (8-OH-DPAT 1 mg kg⁻¹, i.p.) resulted in decrease of extracellular 5-hydroxytryptamine (5-HT). The decrease of the 5-HT levels in the miCure-135-3-treated groups was significantly shorter compared with the levels in the control group (FIG. 14E) suggesting that the intranasal administration of sertraline-conjugated miR-135 mimetics (e.g. miCure-135-3) decreases the 5-HT1A-auto receptor in the dorsal raphe nucleus. The same experimental group, i.e. mice that were administrated once intranasally with 2500 μg of miCure-135-3 or with a conjugated control were tested in the tail suspension test. The results revealed that mice treated with miCure-135-3 showed a significant reduction in immobility time compered to control group (FIG. 14G) which indicates the anti-depressive like effect of sertraline-conjugated miR-135 mimetics (e.g. miCure-135-3).

### Example 11

### Acute Intranasal Administration of Sertraline Conjugated miR-135 Mimetics Decreased the Depressive-Like Behavior in Mice that were Previously Subjected to Depression-Like Inducing Protocol

The potential of sertraline-conjugated miR-135 mimetics to serve as anti-depressant agents was demonstrated using a depression-like mice model. Mice were treated with corticosterone in their drinking water and were subjected to restrained stress for 2 hours every days for 28 days. Mice subjected to the paradigm displayed increased depression-like behavior. Following this protocol mice were treated with one dose of miCure-135-3 (2500 μg). An anti-depressive like effect was shown 3 days following the treatment by decreased immobility time at the tail suspension test of miCure-135-3-treated mice compared to controls (FIG. 15).

### Example 12

### Selective Accumulation of miR-135 Mimetics in the Dorsal Raphe Nucleus Following Intranasal Administration

To examine the brain distribution of miR-135 mimetics following intranasal administration, an alexa488-labeled miCure-135-3 was synthetized. The labeled molecule was delivered to mice using intranasal administration and 6 hours following administration brain tissue was collected. A Confocal fluorescence microscopy revealed that alexa488-labeled miCure-135-3 was intracellularly detected in TPH2-positive midbrain 5-HT neurons after intranasal administration (FIGS. 16A-C). Confocal analysis showed that alexa488-labeled miCure-135-3 was absent in cells of brain areas close to the application site (olfactory bulbs) or to brain ventricles (hippocampus and striatum) (FIG. 16D), supporting that surface SERT expression is a requirement for the oligonucleotide uptake and internalization and that miCure-135-3 is accumulated selectively in the DRN.

### Example 13

### miR-135 Mimetic has No Effect on Cellular Viability in the Brain

To further examine the safety of miR-135 mimetics, mice received a single direct injection directly into the dorsal raphe nucleus of a conjugate-control, miCure-135-3 or a positive control that was reported previously to have no effect on cellular viability (Conjugated-sertraline-siRNA against SERT as taught in in Ferrés-Coy et al., Mol. Psychiatr. (2016) 21(3): 328-38). Sections of the midbrain raphe nuclei were stained with neuronal (NeuN-positive), serotonergic neurons (TPH-positive) or, microglial (Iba-1-positive) markers. Immunohistochemistry analysis demonstrated that miCure-135-3 did not induce neuronal degeneration (FIG. 17A), serotonergic neuron degeneration (FIG. 17C) nor immune responses (FIG. 17B). These can also be seen in the representative images of midbrain raphe nuclei stained with NeuN, Iba1 and TPH under the different treatments (FIG. 17D). These data support the specificity and safety of miCure-135-3.

### Example 14

### Assessing the Effect of Acute Intranasal Administration of Sertraline Conjugated miR-135 in a Depression-Like Protocol of Chronic Social Defeat Stress

The effect of sertraline-conjugated miR-135 mimetics as anti-depressant agents are demonstrated using the chronic social defeat stress protocol utilizing aggressive ICR mice [previously described in Krishnan V. et al., Cell (2007) 131:391-404]. Mice subjected to this paradigm display increased depression-like behavior. Mice are treated with one dose of miCure-135-3 (2500 μg) and the anti-depressive like effect is tested 2 and 3 days following treatment by measuring immobility time at the tail suspension test of miCure-135-3-treated mice compared to controls.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition of matter comprising a synthetic miR-135 molecule comprising a nucleic acid sequence of a miR-135b as set forth in SEQ ID NO: 37, and a complementary strand as set forth in SEQ ID NO: 40.

2. The composition of matter of claim 1, wherein said miR-135 molecule comprises no more than 50 nucleic acids.

3. The composition of matter of claim 1, wherein said nucleic acid sequence of said miR-135b as set forth in SEQ ID NO: 37 and said complementary strand as set forth in SEQ ID NO: 40 are 100% complementary over the entire length of SEQ ID NO: 37 and SEQ ID NO: 40.

4. The composition of matter of claim 1, wherein said nucleic acid sequence of said miR-135b and/or said complementary strand comprises one or more modification selected from the group consisting of a sugar modification, a nucleobase modification, and an internucleotide linkage modification.

5. The composition of matter of claim 4, wherein said sugar modification is selected from the group consisting of a 2′-O-methyl (2′-O-Me), a 2′-O-methoxyethyl (2′-O-MOE), a 2′-fluoro (2′-F), a locked nucleic acid (LNA), and a 2′-Fluoroarabinooligonucleotides (FANA).

6. The composition of matter of claim 4, wherein the sugar modification is a 2′-O-methyl (2′-O-Me), a 2′-O-methoxyethyl (2′-O-MOE), and/or a 2′-fluoro (2′-F) modification.

7. The composition of matter of claim 4, wherein said internucleotide linkage modification is selected from the group consisting of a phosphorothioate, a chiral phosphorothioate, a phosphorodithioate, a phosphotriester, an aminoalkyl phosphotriester, a methyl phosphonate, an alkyl phosphonate, a chiral phosphonate, a phosphinate, a phosphoramidate, an aminoalkylphosphoramidate, a thionophosphoramidate, a thionoalkylphosphonate, a thionoalkylphosphotriester, a boranophosphate, a phosphodiester, a phosphonoacetate (PACE) and a peptide nucleic acid (PNA).

8. A composition of matter comprising a synthetic miR-135 molecule comprising a nucleic acid sequence of a miR-135b as set forth in any one of SEQ ID NOs: 10, 16 or 41-46, and a complementary strand as set forth in any one of SEQ ID NOs: 13 or 47.

9. The composition of matter of claim 8, wherein said nucleic acid sequence of said miR-135b is as set forth in SEQ ID NO: 42 and said complementary strand is as set forth in SEQ ID NO: 13.

10. A composition of matter comprising a nucleic acid construct of the synthetic miR-135 molecule of claim 1.

11. A conjugate comprising:
(i) a composition of matter comprising the synthetic miR-135 molecule of claim 1; and
(ii) a cell-targeting moiety.

12. The conjugate of claim 11, wherein said cell-targeting moiety is conjugated to a 5′ end of said nucleic acid sequence of said complementary strand.

13. The conjugate of claim 11, wherein the cell-targeting moiety is conjugated to a 3′ end of the nucleic acid sequence of the complementary strand.

14. The conjugate of claim 11, wherein said synthetic miR-135 molecule and said cell-targeting moiety are connected by a linking group.

15. The conjugate of claim 14, wherein said cell-targeting moiety is selected from the group consisting of a serotonin reuptake inhibitor (SRI), a selective serotonin reuptake inhibitor (SSRI), a serotonin-norepinephrine reuptake inhibitor (SNRI), a noradrenergic and specific serotoninergic antidepressant (NASSA), a noradrenaline reuptake inhibitor (NRI), a dopamine reuptake inhibitor (DRI), an endocannabinoid reuptake inhibitor (eCBRI), an adenosine reuptake inhibitor (AdoRI), an excitatory Amino Acid Reuptake Inhibitor (EAARI), a glutamate reuptake inhibitor (GluRI), a GABA Reuptake Inhibitor (GRI), a glycine Reuptake Inhibitor (GlyRI) and a Norepinephrine-Dopamine Reuptake Inhibitor (NDRI).

16. The conjugate of claim 15, wherein said selective serotonin reuptake inhibitor (SSRI) is selected from the group consisting of sertraline, a sertraline-structural analog, fluoxetine, fluvoxamine, paroxetine, indapline, zimelidine, citalopram, dapoxetine, escitalopram, and mixtures thereof.

17. The conjugate of claim 16, wherein when said cell-targeting moiety is sertraline said conjugate has the structure:

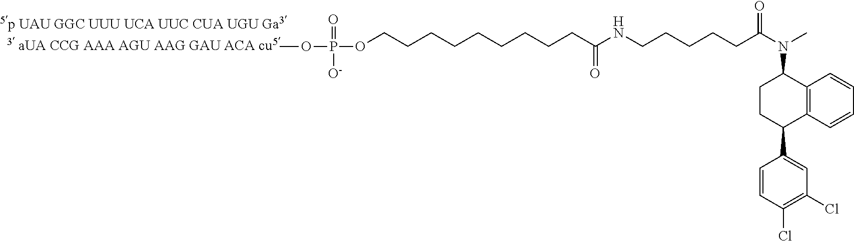 [SMILES: COP(=O)([O-])OCCCCCCCCCC(=O)NCCCCCC(=O)N(C)[C@@H]1CC[C@H](c2ccc(Cl)c(Cl)c2)c2ccccc21]

18. The conjugate of claim 16, wherein when said cell-targeting moiety is sertraline said conjugate has the structure:

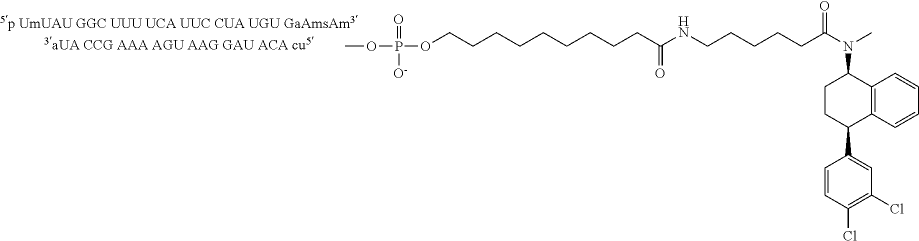 [SMILES: COP(=O)([O-])OCCCCCCCCCC(=O)NCCCCCC(=O)N(C)[C@@H]1CC[C@H](c2ccc(Cl)c(Cl)c2)c2ccccc21]

19. The conjugate of claim 16, wherein when said cell-targeting moiety is sertraline said Conjugate has the structure:

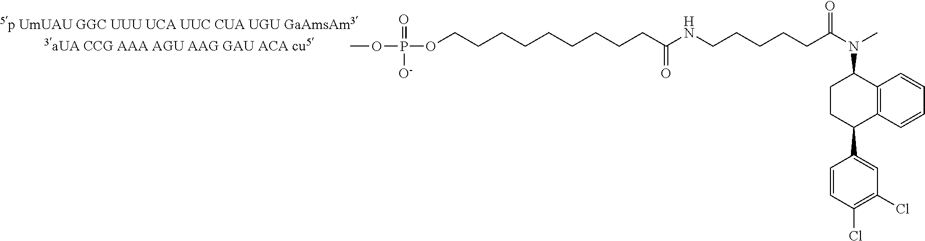 [SMILES: COP(=O)([O-])OCCCCCCCCCC(=O)NCCCCCC(=O)N(C)[C@@H]1CC[C@H](c2ccc(Cl)c(Cl)c2)c2ccccc21]

20. A method of treating a central nervous system (CNS)-related condition, a depression-related disorder or a cancerous disease in a subject in need thereof, the method comprising administering to the subject a therapeutically effective amount of the composition of matter of claim 1, thereby treating the CNS-related condition, the depression-related disorder or the cancerous disease.
